SEQUENCE LISTING (79 sequences)

SEQ ID NO: 1 - DNA, 7260 bp, Homo sapiens
  exon (851)..(1017): exon 1
  Intron (1018)..(1046)
  exon (1047)..(1675): exon 2
  Intron (1676)..(1718)
  exon (1719)..(2371): exon 3
tgttatgtagtcttttgtttggtctctcccttagcataacgatgtttgagatgatgccattcattcatttttgttgctgagcagctgccgagtattgttggaatcccagtttattcattggtttctgtgtctccagttgatagacatgtggattcctccagttagggtttgttattaatgaagccactataaataactgcttacaagtgtggacttacatttttatttcttttggataaatacgtatttgtggaattgctgggccatgtggtaatagatgggtaactgtataagaaactgccataccactttacaaattggctgccacattttttgcattcctaccagcaatatcagacattcctattttttccatattcttgccagtgttaagacttatcatatgtctttttaactttatctgctctaggtgatgtgtgatggtttctcattgtggttttaacttgcacttctttgatgactagtattgtttgctatcttttcatgttcatctaagcgacttattacatatattttatgaactattttgcaaattcaatgattaattccagagactttttcagaattccctagtgttttctacatatacaatgaagttggtgacaaagaaagactttcatttcttcctttcttatccattgatcttttttcttttaaaattattattatttggtagagatgaggtctcacttatcaggctggtctcaaactcctgatctcaagtgatcctcccacctcagcctcccaaaatgcagggattacaggcatgagccaccatgcctggtccttgttgcactggttaggatgactgttaggtgtttaaacaagaatgatgagagctcacatgtttgtttacaaggaacttaaacaaatttacaagaaaaaaacccatccccatcaaaaagtgggcaaaggatataaacagacacttctcagaggaagacatttacgtggccaagaaacatatgaaaaaaagctcacacacgtatatgaaacgtgactgtttataatcctatccaaaaaagaactgatttcaagcaacagcagtattacttccattcaatacttggacctgcaaacatcaaaaaagccactggagaaactgaacgactctctgaaagccttaaactaagatatgaagaagttgaaatctggaaaaaacttgaggaaaaggacaggcagggggaagcacagtggctacaacaaaaaaggcaggaaacaggaagagaggatggcagcatgttggctaaaggttctttggagattgtattggattccaaagacaaaacccaaaagagcaatggtgaaaagaatgaaaaatgtgagaccaaagagaaaggagcaatcacagcaaaggaactatacacaatgatgatggataaaaacatcagcttgattataatggatgctcaaagaatgcaggattatcaggattcctgtattttacattctctcagtgttcctgaaaaagccatcagtccaggagtcactgctagctggattgaagcacacctcccagatgattctatagatacatggaagaagagggggaatgtggagtatatggtacttcttgactggtttagttctgcaaaagatttacagattggaacaacactctggcatctgaaagatgcacttttcaagtgggaaagtaaaactgtcctgtgcaatgggccttgggcctttggttttagagggaggctataaaaactggttcctttgctattcccagtatacaacaaatgctaaggtcactccacccccacaacaccagaatgaagagttgtctatctcattggattttacttatccctcattggaagaatcaattccttctaaacctgctgccgagatgccacctccacctataaaagtggatgaagacatagaattgataagtgatcaaataagtgataatgatcaaaatgagaggacaggaccactgaatatatcaattccagttgaatcagttgctgcttctaaatctgatgtttcacccatcattcagccagtgcctagcataaagaatgttccacagattgatcatactaaaaaactggcagtcaaattgcctgaagagcatataatcaaatctgaaagtacaaatcatgagcaacagtctcctcagaatgaaaaagttattcctgattgttccaccaagccagtagtttcctctccaactctcatgttaacagatgaagaaaaggctcatattcatgcagaaactgctcttctaatggagaaaaacaaacaagaaaaagaacttcaggaaagacagcaagggaaacagaaagaaactgaggagggaagaacacgagcaaaaagccaaaaagaaacaagaagctgaagaaaatgaaattacacagaagcaacaaaaagcaaaagaagaaatggagaagaaagaacgtgaacaggccaagaaagaggataaagaaatctcagcaaagaagggcaaagaaataacaagagtaaaaagacaaagtaaaagtgatcatgaaacctctggtgccgagaagtctgtagaggacagggggagaagatgttcaaccccagaagtacagaaaaagtcaacaagagatgtgtcccatacatctgcgacaggggattcaggttcaggcaagccttttaagattaaaggacaaccagaaactggaattctaaggacagaaacttttagagaggatacagatgatacttaaagaaataaaactcaacgagaaccttcgataatagcacgaagtgaagaaatggggaggatggtaccaggactgccttcaggctgggccaagtttcttgatccaatcactggaacgtttcattattatcattcaccactaacactgttcatatgtacccactggaaatggctccttcatctgcacctccttccacccctccaactcataaaggcaagccacagattcctgctaagcaggatagggaaccttccaaactgaaatgctcttactcctccccagatataacccaggctattcaagaggaagccagcagtaactccaacagttaatcaggaagacaagccaacatgctaccctaaagctgagatctcaaggctttctgcttctcagatttggaaactcaatcctgtttttggaggttctggaccagctcttactggacttcgtaacttaggaaatacttgttatatgaactcaatattgcagtgcctatgtaatcctccacatttggctgattatttcaaccgaaactgttatcaggatgatattaacaagtcaaatttgttaggggcataaaggtgaagtggcagaagaatttggtataatcatgaaagccccgtggacaggacagtatagatatatcagtccaaaagaccttaaagtcaccattgggaagatcaattaccagtttgcaggatacagtcaagattcacaagaatttcttctgttcctaatggatggtctccatgaagatctaaataaaactgataatcggaagacatataaagaagaaaataatgatcatctcaatgactttaaagctgcagaacatgcctggcagaaacacaagcggctctatgagtctattattgttgcactttttcagggtcaattcaaatctacagtacagtgcctcacccgtcacaaaaagtctaggacacttgaggccttcatgtatttgtctctactgatagcatccacaagtaaatgtacattataggattgccttagattattttctaaagaagaaaaactcatagataataacagattttactgcaatctttgcagagctcgacgggattcttaaaaaagaaatctggaagttaccacctgtgcttttagtgcatctgaaacatttttcctacaatggcaggtggaaacaaaaattacagacatctgtggacttcccgttagaaaatcttgccttgtcacagtatgttattggtccaaagaacaatttgaagaaatataatttgttttctgtttcagatcactgcggtgggctggatggaggccattacacagcctactgtaaaaatgcagcaaaacagcggtggtttaagtttgatgatcatgaagtttctgatatctctgtttcttctgtgaaatcttcagcagcttatatcctcttttatacttctttgggaccatgagtaactgatgtaggcacataaggagacataggttataaactagttatcttttaaaaggctcagcaacacaattcttgaaatgcttatcaagatagtggtagcaatagctggccatttagaggaattctaggacagtgggagctgtgttactagcactatataattcctgtcagtggtgacaaataacacttaacaagtattgcagtaagcatcacttacaggtaccatttatttcaaaacaacttttttagtctgctccaaagttaaaataattaactagctaagcattattattctactggtctaaaaacctttgtaccctttttttccttttcactgttacagccttttcacatttctaaatcccatcttcatatactatgaatactctagaatgatgtgaagcagataggaatgtatgtgtacatatttattgcatacttacacatcaaatcgatatacatagtttaacatgtggtcctttcgtgaaacttagaactcagaggattgcatttttttctttgagcatattttgagtaactgcagtgctttcttagggaaatgacagggcaaagctatttttctgttggctttgggggcatttgggtgcgctaaatctttatcttaaaaaataaatggaaacttcctttaattttttaaaatgagacattaaaatcttaatgagaaaaatttaaaaagctcaatatcactgctcattagagaaatgtaaatcaaagccacaatgagataccatctcccaccagtcagaatggtaattattaaaaagtcaagaaacaatagatgctggtgaggctgtggagaaataggaacacttttacactgttgttgggaatgtaaactagttcaaccattgtggaagacagtgtggccattcctcagagatctagaaccagaaatactatttgaccctttgggtatctacccaaaggaatataaatcattctactataaagacacatgcacacgtatgtttactgcagcactatttacaatagcaaagacttggaaccaacccaaatgtccatcagtgatagatggataaagaaaatgtggtgcataccaccatggaatagtacacagccagaaaaaggaatgagttcatgtcctttgcagggacatggatgaagctggaagtcatcatcctcagcaaactaacacgggaacagaaaacaaagcacctcatgttctcattcctaagtgagagttgaacaatgacaacacatggacacagggaggggaacaacacatatcagggccttttggggagtgtggggggcaagggacgagaacttagaggatgggtcaataggtgcagcaaaccaccatggcagactatacgcatgtaacaaacctgcaggttctgcacatgtatcctggaacctaaagtaaaataaaacaaagcaaattaaaaaaagaaagcccatgtcttacatgtatgcatatgttcattgcagcactattcacaatagcaaagacatggaatcaacctaaatgtccatcaatggtagactggataaagaaaatgtggcaaatatgctctaccggcaggatttgatggcgtgatgtctcacagaaagttctccactcccagacatgggtccctcggcttcctgccttggaagcacagcagcaggcatcgtgggaaggtgaagagcttccctaaggatgacccatccaagccggtccacctcacagccttcctgggatacaaggctggcatgacccacatcgtgcgggaagtcgacaggccaggatccaaggtgaacaagaaggaggtggtggaggctgtgaccattgtggagaggccaccagtgggcattgtgggctgcgtggaaacccctcaaggcttccggacttgcaagactgtcttcgctgagcacatcagtgatgaatgcaagaggcgtttctataagaactggcataaatctaagaagaaggcctttaccaagtactgcaagaaatggcaggatgaggatggcaagaagcagctggagaaggacttcagcagcatgaagaagtactgccaagtcatctgcgtcattgcccacacccagatgcacctgcttcctctgtgccagaagaaggcccacctgatggagatccaggtgaatggaggcactgtggctgagaagctggactgggctggcgagaggctcaagcaccaggtacctgtgaaccaagtgtttgggcaggatgagatgatcgacgtcatcagggtgaccaagggcaaaggctacaaaagggtcaccagtcattggcacaccaagaagctgccccgcaagacccaccaaggcctgtgcaaggtggcctgtattggggcatggcatcctgctcgtgtgggcttctctgtggtacgtggtgggcagaaaggctaccatcaccgcactgagatcaacaagatctataggattggctagggctaccttatcaaggatggcaagctgatcaagaacaatgcctccactgactatgacctgtctgacaagagcatcaaccctttgggtggcttcgtccactatggtgaagtgaccaatgactttgtcatgctgaaaggctgtgtggtgggaaccaagaagtgggtgctcaccctccacaagtccttgctggtgcagacaaagcagcgggctctggagaagattgaccttaagttcattgacacctcctccaagtttggccatggccgcttccagaccacggaggagaagaaagcattcatgggaccactcaagaaagaccgaattgcaaaggaagaaggagcttaatgctgggaacagatattgcaactggtgggatctcaataaaagttattttccattaaaaaaaaaagaaaaagaaaatgtggcacatatacaccacagaataccatgcagccataaaaaagaatgagatcatgtcctttgcaggaacatggatggagttggaggccattatccttagcaaactgaggcaggaacagaaaaccaattaccacatgttctcacttataagtaggagttatatgatgagaacacatggacacacagaagggaacaacacacactggggtccacttgagggtagagggtgggaggagggagagg

SEQ ID NO: 2 - DNA, 1449 bp, Homo sapiens
  CDS (1)..(1449)
  misc_feature (1)..(167): exon 1
  misc_feature (168)..(796): exon 2
  misc_feature (797)..(1449): exon 3
atgatgagagctcacatgtttgtttacaaggaacttaaacaaatttacaagaaaaaaacccatccccatcaaaaagtgggcaaaggatataaacagacacttctcagaggaagacatttacgtggccaagaaacatatgaaaaaaagctcacacacgtatatgaaacaactgatttcaagcaacagcagtattacttccattcaatacttggacctgcaaacatcaaaaaagccactggagaaactgaacgactctctgaaagccttaaactaagatatgaagaagttgaaatctggaaaaaacttgaggaaaaggacaggcagggggaagcacagtggctacaacaaaaaaggcaggaaacaggaagagaggatggcagcatgttggctaaaggttctttggagattgtattggattccaaagacaaaacccaaaagagcaatggtgaaaagaatgaaaaatgtgagaccaaagagaaaggagcaatcacagcaaaggaactatacacaatgatgatggataaaaacatcagcttgattataatggatgctcaaagaatgcaggattatcaggattcctgtattttacattctctcagtgttcctgaaaaagccatcagtccaggagtcactgctagctggattgaagcacacctcccagatgattctatagatacatggaagaagagggggaatgtggagtatatggtacttcttgactggtttagttctgcaaaagatttacagattggaacaacactctggcatctgaaagatgcacttttcaagtgggaaaagggaggctataaaaactggttcctttgctattcccagtatacaacaaatgctaaggtcactccacccccacaacaccagaatgaagagttgtctatctcattggattttacttatccctcattggaagaatcaattccttctaaacctgctgccgagatgccacctccacctataaaagtggatgaagacatagaattgataagtgatcaaataagtgataatgatcaaaatgagaggacaggaccactgaatatatcaattccagttgaatcagttgctgcttctaaatctgatgtttcacccatcattcagccagtgcctagcataaagaatgttccacagattgatcatactaaaaaactggcagtcaaattgcctgaagagcatataatcaaatctgaaagtacaaatcatgagcaacagtctcctcagaatgaaaaagttattcctgattgttccaccaagccagtagtttcctctccaactctcatgttaacagatgaagaaaaggctcatattcatgcagaaactgctcttctaatggagaaaaacaaacaagaaaaagaacttcaggaaagacagcaagggaaacagaaagaaactgaggagggaagaacacgagcaaaaagccaaaaagaaacaagaagctga

SEQ ID NO: 3 - protein, 482 aa, Homo sapiens
MMRAHMFVYKELKQIYKKKTHPHQKVGKGYKQTLLRGRHLRGQETYEKKLTHVYETTDFKQQQYYFHSILGPANIKKATGETERLSESLKLRYEEVEIWKKLEEKDRQGEAQWLQQKRQETGREDGSMLAKGSLEIVLDSKDKTQKSNGEKNEKCETKEKGAITAKELYTMMMDKNISLIIMDAQRMQDYQDSCILHSLSVPEKAISPGVTASWIEAHLPDDSIDTWKKRGNVEYMVLLDWFSSAKDLQIGTTLWHLKDALFKWEKGGYKNWFLCYSQYTTNAKVTPPPQHQNEELSISLDFTYPSLEESIPSKPAAEMPPPPIKVDEDIELISDQISDNDQNERTGPLNISIPVESVAASKSDVSPIIQPVPSIKNVPQIDHTKKLAVKLPEEHIIKSESTNHEQQSPQNEKVIPDCSTKPVVSSPTLMLTDEEKAHIHAETALLMEKNKQEKELQERQQGKQKETEEGRTRAKSQKETRS

SEQ ID NO: 4 - protein, 1118 aa, Homo sapiens
MPAVASVPKELYLSSSLKDLNKKTEVKPEKISTKSYVHSALKIFKTAEECRLDRDEERAYVLYMKYVTVYNLIKKRPDFKQQQDYFHSILGPGNIKKAVEEAERLSESLKLRYEEAEVRKKLEEKDRQEEAQRLQQKRQETGREDGGTLAKGSLENVLDSKDKTQKSNGEKNEKCETKEKGAITAKELYTMMTDKNISLIIMDARRMQDYQDSCILHSLSVPEEAISPGVTASWIEAHLPDDSKDTWKKRGNVEYVVLLDWFSSAKDLQIGTTLRSLKDALFKWESKTVLRNEPLVLEGGYENWLLCYPQYTTNAKVTPPPRRQNEEVSISLDFTYPSLEESIPSKPAAQTPPASIEVDENIELISGQNERMGPLNISTPVEPVAASKSDVSPIIQPVPSIKNVPQIDRTKKPAVKLPEEHRIKSESTNHEQQSPQSGKVIPDRSTKPVVFSPTLMLTDEEKARIHAETALLMEKNKQEKELRERQQEEQKEKLRKEEQEQKAKKKQEAEENEITEKQQKAKEEMEKKESEQAKKEDKETSAKRGKEITGVKRQSKSEHETSDAKKSVEDRGKRCPTPEIQKKSTGDVPHTSVTGDSGSGKPFKIKGQPESGILRTGTFREDTDDTERNKAQREPLTRARSEEMGRIVPGLPSGWAKFLDPITGTFRYYHSPTNTVHMYPPEMAPSSAPPSTPPTHKAKPQIPAERDREPSKLKRSYSSPDITQAIQEEEKRKPTVTPTVNRENKPTCYPKAEISRLSASQIRNLNPVFGGSGPALTGLRNLGNTCYMNSILQCLCNAPHLADYFNRNCYQDDINRSNLLGHKGEVAEEFGIIMKALWTGQYRYISPKDFKITIGKINDQFAGYSQQDSQELLLFLMDGLHEDLNKADNRKRYKEENNDHLDDFKAAEHAWQKHKQLNESIIVALFQGQFKSTVQCLTCHKKSRTFEAFMYLSLPLASTSKCTLQDCLRLFSKEEKLTDNNRFYCSHCRARRDSLKKIEIWKLPPVLLVHLKRFSYDGRWKQKLQTSVDFPLENLDLSQYVIGPKNNLKKYNLFSVSNHYGGLDGGHYTAYCKNAARQRWFKFDDHEVSDISVSSVKSSAAYILFYTSLGPRVTDVAT

SEQ ID NO: 5 - DNA, 29 bp, Artificial
  /note=“Description of artificial sequence primer”
atgatgagagctcacatgtttgtttacaa

SEQ ID NO: 6 - DNA, 24 bp, Artificial
  /note=“Description of artificial sequence primer”
tttcaagaattgtgttgctgagcc

SEQ ID NO: 7 - DNA, 30 bp, Artificial
  /note=“Description of artificial sequence primer”
gctcacatgtttgtttacaaggaacttaaa

SEQ ID NO: 8 - DNA, 26 bp, Artificial
  /note=“Description of artificial sequence primer”
tgcctacatcagttactcatggtccc

SEQ ID NO: 9 - DNA, 22 bp, Artificial
  /note=“Description of artificial sequence primer”
gtttgtttacaaggaacttaaa

SEQ ID NO: 10 - DNA, 19 bp, Artificial
  /note=“Description of artificial sequence primer”
gatttcaagcaacagcagt

SEQ ID NO: 11 - DNA, 20 bp, Artificial
  /note=“Description of artificial sequence primer”
gaaagatgcacttttcaagt

SEQ ID NO: 12 - DNA, 22 bp, Artificial
  /note=“Description of artificial sequence primer”
aagagttgtctatctcattgga

SEQ ID NO: 13 - DNA, 22 bp, Artificial
  /note=“Description of artificial sequence primer”
gaccatgagtaactgatgtagg

SEQ ID NO: 14 - DNA, 19 bp, Artificial
  /note=“Description of artificial sequence primer”
catggtcccaaagaagtat

SEQ ID NO: 15 - DNA, 17 bp, Artificial
  /note=“Description of artificial sequence primer”
aaaccaccgctgttttg

SEQ ID NO: 16 - DNA, 21 bp, Artificial
  /note=“Description of artificial sequence primer”
tgaccttagcatttgttgtat

SEQ ID NO: 17 - DNA, 20 bp, Artificial
  /note=“Description of artificial sequence primer”
cttgaaaagtgcatctttca

SEQ ID NO: 18 - DNA, 18 bp, Artificial
  /note=“Description of artificial sequence primer”
ctgcctgtccttttcctc

SEQ ID NO: 19 - DNA, 21 bp, Artificial
  /note=“Description of artificial sequence primer”
gtgtgagctttttttcatatg

SEQ ID NO: 20 - DNA, 20 bp, Artificial
  /note=“Description of artificial sequence primer”
cgtaaatgtcttcctctgag

SEQ ID NO: 21 - DNA, 30 bp, Artificial
  /note=“Description of artificial sequence primer”
aagtgataatgatcaaaatgagaggacagg

SEQ ID NO: 22 - DNA, 24 bp, Artificial
  /note=“Description of artificial sequence primer”
tgttccaccaagccagtagtttcc

SEQ ID NO: 23 - DNA, 19 bp, Artificial
  /note=“Description of artificial sequence primer”
acatcaaaaaagccactgg

SEQ ID NO: 24 - DNA, 19 bp, Artificial
  /note=“Description of artificial sequence primer”
cactgctagctggattgaa

SEQ ID NO: 25 - DNA, 22 bp, Artificial
  /note=“Description of artificial sequence primer”
cctcccagatgattctatagat

SEQ ID NO: 26 - DNA, 22 bp, Artificial
  /note=“Description of artificial sequence primer”
cacctataaaagtggatgaaga

SEQ ID NO: 27 - DNA, 21 bp, Artificial
  /note=“Description of artificial sequence primer”
cagtagtttcctctccaactc

SEQ ID NO: 28 - DNA, 19 bp, Artificial
  /note=“Description of artificial sequence primer”
gaggagggaagaacacgag

SEQ ID NO: 29 - DNA, 26 bp, Artificial
  /note=“Description of artificial sequence primer”
gatacttaaagaaataaaactcaacg

SEQ ID NO: 30 - DNA, 19 bp, Artificial
  /note=“Description of artificial sequence primer”
ttccaaactgaaatgctct

SEQ ID NO: 31 - DNA, 17 bp, Artificial
  /note=“Description of artificial sequence primer”
tgcccttctttgctgag

SEQ ID NO: 32 - DNA, 22 bp, Artificial
  /note=“Description of artificial sequence primer”
tgactgccagttttttagtatg

SEQ ID NO: 33 - DNA, 19 bp, Artificial
  /note=“Description of artificial sequence primer”
atctttcagatgccagagt

SEQ ID NO: 34 - DNA, 18 bp, Artificial
  /note=“Description of artificial sequence primer”
cctttagccaacatgctg

SEQ ID NO: 35 - DNA, 19 bp, Artificial
  /note=“Description of artificial sequence primer”
tcgttcagtttctccagtg

SEQ ID NO: 36 - DNA, 22 bp, Artificial
  /note=“Description of artificial sequence primer”
cgagatgccacctccacctata

SEQ ID NO: 37 - DNA, 23 bp, Artificial
  /note=“Description of artificial sequence primer”
tcagtggtcctgtcctctcattt

SEQ ID NO: 38 - DNA, 23 bp, Artificial
  /note=“Description of artificial sequence primer”
tgatcaaataagtgataatgatc

SEQ ID NO: 39 - DNA, 26 bp, Artificial
  /note=“Description of artificial sequence primer”
aattgatatattcagtggtcctgtcc

SEQ ID NO: 40 - DNA, 21 bp, Artificial
  /note=“Description of artificial sequence primer”
cgagatgccacctccacctat

SEQ ID NO: 41 - DNA, 21 bp, Artificial
  /note=“Description of artificial sequence primer”
tctcattttgatcattatcac

SEQ ID NO: 42 - DNA, 19 bp, Artificial
  /note=“Description of artificial sequence primer”
agacaagccaacatgctac

SEQ ID NO: 43 - DNA, 21 bp, Artificial
  /note=“Description of artificial sequence primer”
tggtgactttaaggtcttttg

SEQ ID NO: 44 - DNA, 20 bp, Artificial
  /note=“Description of artificial sequence primer”
ctcacccgtcacaaaaagtc

SEQ ID NO: 45 - DNA, 20 bp, Artificial
  /note=“Description of artificial sequence primer”
gcttcttgccatcctcatcc

SEQ ID NO: 46 - DNA, 19 bp, Artificial
  /note=“Description of artificial sequence primer”
cctgtgcttttagtgcatc

SEQ ID NO: 47 - DNA, 20 bp, Artificial
  /note=“Description of artificial sequence primer”
gctgaagatttcacagaaga

SEQ ID NO: 48 - DNA, 22 bp, Artificial
  /note=“Description of artificial sequence primer”
ctttcgtgaaacttagaactca

SEQ ID NO: 49 - DNA, 20 bp, Artificial
  /note=“Description of artificial sequence primer”
cattgtggctttgatttaca

SEQ ID NO: 50 - DNA, 20 bp, Artificial
  /note=“Description of artificial sequence primer”
gaaacaatagatgctggtga

SEQ ID NO: 51 - DNA, 15 bp, Artificial
  /note=“Description of artificial sequence primer”
actgatggacatttg

SEQ ID NO: 52 - DNA, 1661 bp, Homo sapiens
  CDS (1)..(1455)
  misc_feature: First splice variant of allele A9.1
atgatgagagctcacatgtttgtttacaaggaacttaaacaaatttacaagaaaaaaacccatccccatcaaaaagtgggcaaaggatataaacagacacttctcagaggaagacatttacgtggccaagaaacatatgaaaaaaagctcacacacgtatatgaaacacctgatttcaagcaacagcaggattgcttccgttcaatacttggacctgcaaacatcaaaaaagccactggagaaactgaacgactctctgaaagccttaaactaagatatgaagaagttgaaatctggaaaaaacttgaggaaaaggacaggcagggggaagcacagtggctacaacaaaaaaggcaggaaacaggaagagaggatggcagcacgttggctaaagattctttggagattgtattggattccaaagacaaaacccaaaagagcaatggtgaaaagaatgaaaaatgtgagaccaaagagaaaggagcaatcacagcaaaggaactatacacaatgatgatggataaaaacatcagcttgattataatgcatgctcaaagaatgcagtattatcaggattcctgtattttacattctctcagtgttcctgaaaaagccatcagtccaggagtcactgctagctggattgaagcacacctcccagatgattctatagatacatggaagaagagggggaatgtggagtatatggtacttcttgactggtttagttctgcaaaagatttacagattggaacaacactctggcatctgaaagatgcacttttcaagtgggaaaagggaggctataaaaactggttcttttgctattcccagtatacaacaaatgctaaggtcactccacccccacaacaccagaatgaagagttgtctatctcattggattttacttatccctcattggaagaatcaattccttctaaacctgctgccgagatgccacctccacctatagaagtggatgaagacatagaattgataagtgatcaaataagtgataatgatcaaaatgagaggacaggaccactgaatatatcaattccagttgaatcagttgctgcttctaaatctgatgtttcacccatcattcagccagtgcctagcataaagaatgttccacagattgatcatactaaaaaactggcagtcaaattgcctgaagagcatataatcaaatctgaaagtacaaatcatgagcaacagtctcctcagaatgaaaaagttattcctgattgttccgccaagccagtagtttcctctccaactctcatgttaacagatgaagaaaaggctcatattcatgcagaaactgctcttctaatggagaaaaacaaacaagaaaaagaacttcaggaaagacagcaagggaaacagaaagaaactgaggagggaagaacacgagcaaaaagccaaaaagaaacaagaagctgcagaaaatgaaattacacagaagcaacaaaaagcaaaagaagaaatggagaagaaagaacgtgaacaggccaagaaagaggataaagaaatctcagcaaagaagggcaaagaaataacaagagtaaaaagacaaagtaaaagtgatcatgaaacctctggtgccgagaagtctgtagaggacagggggagaagatgttcaaccccagaagtac

SEQ ID NO: 53 - protein, 485 aa, Homo sapiens
MMRAHMFVYKELKQIYKKKTHPHQKVGKGYKQTLLRGRHLRGQETYEKKLTHVYETPDFKQQQDCFRSILGPANIKKATGETERLSESLKLRYEEVEIWKKLEEKDRQGEAQWLQQKRQETGREDGSTLAKDSLEIVLDSKDKTQKSNGEKNEKCETKEKGAITAKELYTMMMDKNISLIIMHAQRMQYYQDSCILHSLSVPEKAISPGVTASWIEAHLPDDSIDTWKKRGNVEYMVLLDWFSSAKDLQIGTTLWHLKDALFKWEKGGYKNWFFCYSQYTTNAKVTPPPQHQNEELSISLDFTYPSLEESIPSKPAAEMPPPPIEVDEDIELISDQISDNDQNERTGPLNISIPVESVAASKSDVSPIIQPVPSIKNVPQIDHTKKLAVKLPEEHIIKSESTNHEQQSPQNEKVIPDCSAKPVVSSPTLMLTDEEKAHIHAETALLMEKNKQEKELQERQQGKQKETEEGRTRAKSQKETRSCRK

SEQ ID NO: 54 - DNA, 1735 bp, Homo sapiens
  misc_feature: Second splice variant of allele A9.1
atgatgagagctcacatgtttgtttacaaggaacttaaacaaatttacaagaaaaaaacccatccccatcaaaaagtgggcaaaggatataaacagacacttctcagaggaagacatttacgtggccaagaaacatatgaaaaaaagctcacacacgtatatgaaacgtgactgtttataatcctatccaaaaaagacctgatttcaagcaacagcaggattgcttccgttcaatacttggacctgatttcaagcaacagcaggattgcttccgttcaatacttggacctgcaaacatcaaaaaagccactggagaaactgaacgactctctgaaagccttaaactaagatatgaagaagttgaaatctggaaaaaacttgaggaaaaggacaggcagggggaagcacagtggctacaacaaaaaaggcaggaaacaggaagagaggatggcagcacgttggctaaagattctttggagattgtattggattccaaagacaaaacccaaaagagcaatggtgaaaagaatgaaaaatgtgagaccaaagagaaaggagcaatcacagcaaaggaactatacacaatgatgatggataaaaacatcagcttgattataatgcatgctcaaagaatgcagtattatcaggattcctgtattttacattctctcagtgttcctgaaaaagccatcagtccaggagtcactgctagctggattgaagcacacctcccagatgattctatagatacatggaagaagagggggaatgtggagtatatggtacttcttgactggtttagttctgcaaaagatttacagattggaacaacactctggcatctgaaagatgcacttttcaagtgggaaaagggaggctataaaaactggttcttttgctattcccagtatacaacaaatgctaaggtcactccacccccacaacaccagaatgaagagttgtctatctcattggattttacttatccctcattggaagaatcaattccttctaaacctgctgccgagatgccacctccacctatagaagtggatgaagacatagaattgataagtgatcaaataagtgataatgatcaaaatgagaggacaggaccactgaatatatcaattccagttgaatcagttgctgcttctaaatctgatgtttcacccatcattcagccagtgcctagcataaagaatgttccacagattgatcatactaaaaaactggcagtcaaattgcctgaagagcatataatcaaatctgaaagtacaaatcatgagcaacagtctcctcagaatgaaaaagttattcctgattgttccgccaagccagtagtttcctctccaactctcatgttaacagatgaagaaaaggctcatattcatgcagaaactgctcttctaatggagaaaaacaaacaagaaaaagaacttcaggaaagacagcaagggaaacagaaagaaactgaggagggaagaacacgagcaaaaagccaaaaagaaacaagaagctgcagaaaatgaaattacacagaagcaacaaaaagcaaaagaagaaatggagaagaaagaacgtgaacaggccaagaaagaggataaagaaatctcagcaaagaagggcaaagaaataacaagagtaaaaagacaaagtaaaagtgatcatgaaacctctggtgccgagaagtctgtagaggacagggggagaagatgttcaaccccagaagtac

SEQ ID NO: 55 - DNA, 1238 bp, Homo sapiens
  misc_feature: Third splice variant of allele A9.1
  CDS (1)..(1032)
atgatgagagctcacatgtttgtttacaaggaacttaaacaaatttacaagaaaaaaacccatccccatcaaaaagtgggcaaaggatataaacagacacttctcagaggaagacatttacgtggccaagaaacatatgaaaaaaagctcacacacgtatatgaaacacctgatttcaagcaacagcaggattgcttccgttcaatacttggacctgcaaacatcaaaaaagccactggagaaactgaacgactctctgaaagccttaaactaagatatgaagaagttgaaatctggaaaaaacttgaggaaaaggacaggcagggggaagcacagtggctacaacaaaaaaggcaggaaacaggaagagaggatggcagcacgttggctaaagattctttggagattgtattggattccaaagacaaaacccaaaagagcaatggtgaaaagaatgaaaaatgtgagaccaaagagaaaggagcaatcacagcaaaggaactatacacaatgatgatggataaaaacatcagcttgattataatgcatgctcaaagaatgcagtattatcaggattcctgtattttacattctctcagtgttcctgaaaaagccatcagtccaggagtcactgctagctggattgaagcacacctcccagatgattctatagatacatggaagaagagggggaatgtggagtatatggtacttcttgactggtttagttctgcaaaagatttacagattggaacaacactctggcatctgaaagatgcacttttcaagtgggaaaagtctcctcagaatgaaaaagttattcctgattgttccgccaagccagtagtttcctctccaactctcatgttaacagatgaagaaaaggctcatattcatgcagaaactgctcttctaatggagaaaaacaaacaagaaaaagaacttcaggaaagacagcaagggaaacagaaagaaactgaggagggaagaacacgagcaaaaagccaaaaagaaacaagaagctgcagaaaatgaaattacacagaagcaacaaaaagcaaaagaagaaatggagaagaaagaacgtgaacaggccaagaaagaggataaagaaatctcagcaaagaagggcaaagaaataacaagagtaaaaagacaaagtaaaagtgatcatgaaacctctggtgccgagaagtctgtagaggacagggggagaagatgttcaaccccagaagtac

SEQ ID NO: 56 - protein, 344 aa, Homo sapiens
MMRAHMFVYKELKQIYKKKTHPHQKVGKGYKQTLLRGRHLRGQETYEKKLTHVYETPDFKQQQDCFRSILGPANIKKATGETERLSESLKLRYEEVEIWKKLEEKDRQGEAQWLQQKRQETGREDGSTLAKDSLEIVLDSKDKTQKSNGEKNEKCETKEKGAITAKELYTMMMDKNISLIIMHAQRMQYYQDSCILHSLSVPEKAISPGVTASWIEAHLPDDSIDTWKKRGNVEYMVLLDWFSSAKDLQIGTTLWHLKDALFKWEKSPQNEKVIPDCSAKPVVSSPTLMLTDEEKAHIHAETALLMEKNKQEKELQERQQGKQKETEEGRTRAKSQKETRSCRK

SEQ ID NO: 57 - protein, 13 aa, Artificial
  Immunigenic peptide
QISDNDQNERTGP

SEQ ID NO: 58 - DNA, 18 bp, Artificial
  /note=“Description of artificial sequence primer”
gccacagcaggcatgatg

SEQ ID NO: 59 - DNA, 15 bp, Artificial
  /note=“Description of artificial sequence primer”
caagtgcttctttcc

SEQ ID NO: 60 - DNA, 20 bp, Artificial
  /note=“Description of artificial sequence primer”
tgcccatctcccaagaactc

SEQ ID NO: 61 - DNA, 23 bp, Artificial
  /note=“Description of artificial sequence primer”
atgaaggagaacttccccaactt

SEQ ID NO: 62 - DNA, 18 bp, Artificial
  /note=“Description of artificial sequence primer”
cttagtgcctgtgacaaa

SEQ ID NO: 63 - DNA, 20 bp, Artificial
  /note=“Description of artificial sequence primer”
caaagacgtcggcgaggtaa

SEQ ID NO: 64 - DNA, 20 bp, Artificial
  /note=“Description of artificial sequence primer”
gtcatcctggagatcgacaa

SEQ ID NO: 65 - DNA, 17 bp, Artificial
  /note=“Description of artificial sequence primer”
gccaggctggctgcgga

SEQ ID NO: 66 - DNA, 20 bp, Artificial
  /note=“Description of artificial sequence primer”
gggccagctcattctcatac

SEQ ID NO: 67 - DNA, 26 bp, Artificial
  /note=“Description of artificial sequence primer”
agatgaggtctcacttatcaggctgg

SEQ ID NO: 68 - DNA, 27 bp, Artificial
  /note=“Description of artificial sequence primer”
tctgtacttctggggttgaacatcttc

SEQ ID NO: 69 - DNA, 23 bp, Artificial
  /note=“Description of artificial sequence primer”
catgatgagagctcacatgtttg

SEQ ID NO: 70 - DNA, 19 bp, Artificial
  /note=“Description of artificial sequence primer”
gccactggagaaactgaac

SEQ ID NO: 71 - DNA, 19 bp, Artificial
  /note=“Description of artificial sequence primer”
gcaatcacagcaaaggaac

SEQ ID NO: 72 - DNA, 19 bp, Artificial
  /note=“Description of artificial sequence primer”
tttggttttagagggaggc

SEQ ID NO: 73 - DNA, 20 bp, Artificial
  /note=“Description of artificial sequence primer”
taaatctgatgtttcaccca

SEQ ID NO: 74 - DNA, 19 bp, Artificial
  /note=“Description of artificial sequence primer”
gttcctttgctgtgattgc

SEQ ID NO: 75 - DNA, 19 bp, Artificial
  /note=“Description of artificial sequence primer”
acatcaaaaaagccactgg

SEQ ID NO: 76 - DNA, 19 bp, Artificial
  /note=“Description of artificial sequence primer”
tcgttcagtttctccagtg

SEQ ID NO: 77 - DNA, 20 bp, Artificial
  /note=“Description of artificial sequence primer”
gaaagatgcacttttcaagt

SEQ ID NO: 78 - DNA, 33 bp, Artificial
  /note=“Description of artificial sequence primer”
gatagatctgtacttctggggttgaacatcttc

SEQ ID NO: 79 - DNA, 31 bp, Artificial
  /note=“Description of artificial sequence primer”
tctggatccatgatgagagctcacatgtttg

(19) United States
(12) Patent Application Publication
(10) Pub. No.: US 2007/0087983 A1
(43) Pub. Date: Apr. 19, 2007

(54) Novel ubp8rp polypeptides and their use in the treatment of psoriasis

(75) Inventors: Ilya Chumakov, Vaux-le Penil (FR); Oxana Guerassimenko, Milly-La-Foret (FR)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: APPLIED RESEARCH SYSTEMS ARS HOLDING N.V.

(21) Appl. No.: 10/570,121

(22) PCT Filed: Sep. 2, 2004
(86) PCT No.: PCT/EP04/52000

§ 371 Date: Feb. 28, 2006

Related U.S. Application Data
(60) Provisional application No. 60/501,201, filed on Sep. 8, 2003.

(30) Foreign Application Priority Data
Sep. 4, 2003 (EP) 03102699.0

(51) Int. Cl. G01N 33/53; A61K 31/711; C12Q 1/68; C07H 21/04; C12N 9/64; A61K 48/00; C12P 21/06

(52) U.S. Cl. 514/44; 435/7.1; 435/6; 435/69.1; 435/320.1; 435/226; 435/325; 530/388.26; 536/23.2

(57) ABSTRACT

The invention encompasses a novel gene encoding a protein of the ubiquitin-proteasome pathway, UBP8rp. The invention also relates the use of UBP8rp polypeptides for screening for modulators, and to the use of said modulators for treating chronic inflammatory diseases such as, e.g., psoriasis, psoriatic arthritis, rheumatoid arthritis, asthma, inflammatory bowel disease and multiple sclerosis. The invention further relates to the use of biallelic markers located in the UBP8rp gene for diagnosing said chronic inflammatory diseases.

### FIELD OF THE INVENTION

This Invention relates to a novel gene encoding a protein of the ubiquitin-proteasome pathway, UBP8rp. The invention also relates the use of UBP8rp polypeptides for screening for modulators, and to the use of said modulators for treating chronic inflammatory diseases such as, e.g., psoriasis, psoriatic arthritis, rheumatoid arthritis, asthma, inflammatory bowel disease and multiple sclerosis. The invention further relates to the use of biallelic markers located in the UBP8rp gene for diagnosing said chronic inflammatory diseases.

### BACKGROUND

1. Psoriasis

Psoriasis is a chronic, recurring disease recognizable by silvery scaling bumps and various—sized plaques (raised patches). An abnormally high rate of growth and turnover of skin cells causes the scaling. The reason for the rapid cell growth is unknown, but immune mechanisms are thought to play a role. The condition often runs in families. Psoriasis is common, affecting 2 to 4% of whites, blacks are less likely to get the disease. Psoriasis begins most often in people aged 10 to 40, although people in all age groups are susceptible.

1.1. Symptoms

Psoriasis usually starts as one or more small psoriatic plaques that be come excessively flaky. Small bumps may develop around the area. Although the first plaques may clear up by themselves, others may soon follow. Some plaques may remain thumbnail-sized, but others may grow to cover large areas of the body, sometimes in striking ring-shaped or spiral patterns.

Psoriasis typically involves the scalp, elbows, knees, back, and buttocks. The flaking may be mistaken for severe dandruff, but the patchy nature of psoriasis, with flaking areas interspersed among completely normal ones, distinguishes the disease from dandruff. Psoriasis can also break out around and under the nails, making them thick and deformed. The eyebrows, armpits, navel, and groin may also be affected. Usually, psoriasis produces only flaking. Even itching is uncommon. When flaking areas heal, the skin takes on a completely normal appearance, and hair growth is unchanged. Most people with limited psoriasis suffer few problems beyond the flaking, although the skin's appearance may be embarrassing.

Some people, however, have extensive psoriasis or experience serious effects from psoriasis. Psoriatic arthritis produces symptoms very similar to those of rheumatoid arthritis. Rarely, psoriasis covers the entire body and produces exfoliative psoriatic dermatitis, in which the entire skin becomes inflamed. This form of psoriasis is serious because, like a burn, It keeps the skin from serving as a protective barrier against injury and infection. In another uncommon form of psoriasis, pustular psoriasis, large and small pus-filled pimples (pustules) form on the palms of the hands and soles of the feet. Sometimes, these pustules are scattered on the body.

Psoriasis may flare up for no apparent reason, or a flare-up may result from severe sunburn, skin irritation, antimalaria drugs, lithium, beta-blocker drugs (such as propranolol and metoprolol), or almost any medicated ointment or cream. Streptococcal infections (especially in children), bruises, and scratches can also stimulate the formation of new plaques.

1.2. Molecular Basis

Psoriasis is a chronic Inflammatory disease. The inflammatory events in psoriasis are composed of a complex series of inductive and effector processes, which require the regulated expression of various proinflammatory genes. NF-κB is a protein transcription factor that is required for maximal transcription of many of these proinflammatory molecules. It consists of a heterodimer of the p50 and p65 proteins retained inactive in the cytoplasm tightly bound to the inhibitory subunit IκB. Upon activation, IκB is rapidly and sequentially phosphorylated by the action of IκB kinases, ubiquitinated, and degraded by the ubiquitin-proteasome. The active subunit (p50 and p65) is translocated to the nucleus, where it binds to cognate DNA sequences and stimulates gene transcription of proinflammatory genes.

1.3. Diagnosis

Psoriasis may be misdiagnosed at first because many other disorders can produce similar plaques and flaking. To confirm a diagnosis, a doctor may perform a skin biopsy by removing a skin specimen and examining it under a microscope.

1.4. Treatment

When a person has only a few small plaques, using ointments and creams that lubricate the skin (emollients) once or twice a day can keep the skin moist. Ointments containing corticosteroids, Vitamin D cream, salicylic add or coal tar are effective in many patients with limited psoriasis. Stronger medications like anthralin are used sometimes, but they can irritate the skin and stain sheets and clothing. When the scalp is affected, shampoos containing these active ingredients are often used. For pustular psoriasis, the two most effective medications are etretinate and isotretinoin, which are also used to treat severe acne. Ultraviolet light also can help clear up psoriasis. In fact, during summer months, exposed regions of affected skin may clear up spontaneously. Sunbathing often helps to clear up the plaques on larger areas of the body: exposure to ultraviolet light under controlled conditions is another common therapy.

No drug for treating severe forms of psoriasis without severe side effects is marketed yet. For extensive psoriasis, ultraviolet therapy may be supplemented by psoralens, drugs that make the skin extra sensitive to the effects of ultraviolet light. The combination of psoralens and ultraviolet light (PUVA) is usually effective and may clear up the skin for several months. However, PUVA treatment can increase the risk of skin cancer from ultraviolet light; therefore, the treatment must be closely supervised by a doctor. For most serious forms of psoriasis and widespread psoriasis, a doctor may give methotrexate. Used to treat some forms of cancer, this drug interferes with the growth and multiplication of skin cells. It can be effective in extreme cases but may cause adverse effects on the bone marrow, kidneys, and liver. Another effective medication, cyclosporine, also has serious side effects.

New generation drugs that are currently under development include Efalizumab (Raptiva®), an humanized anti-CD11a antibody. It has been shown that Efalizumab, given subcutaneously once-weekly, provides clinical benefit in patients with moderate-to-severe plaque psoriasis (Cather et al. (2003) Expert Opin Biol Ther. 3:361-370). Efalizumab offers an new therapeutic option for the treatment of psoriasis and the potential for improved and potentially safer long-term, continuous “maintenance” therapy.

2. Psoriasis Susceptibility Loci

The multifactorial etiology of psoriasis is well established. Although environmental factors, such as streptococcal infections, affect the onset of the disease, family studies indicate a strong genetic component. Twin studies show the concordance in monozygotic twins to be 65 to 70% (Farber et al., 1974), compared to 15 to 20% In dizygotic twins. Family studies estimate the risk to first-degree relatives at between 8 to 23%. However, there are also several known environmental factors, including streptococcal infection and stress, that affect the onset and presentation of the disease.

Several psoriasis susceptibility loci have been mapped: PSORS1 on 6p21, PSORS2 on 17q, PSORS3 on 4q, PSORS4 on 1cen-q21, PSORS5 on 3q21, PSORS6 on 19p, PSORS7 on 1p, and PSORS8 on 4q31. The loci on 6p and 17q appear to be well established. Additional putative psoriasis candidate loci have been reported on 16q and 20p.

The major susceptibility locus for psoriasis is PSORS1 (Nair et al. 1997; Trembath et al. 1997; Oka et al. 1999; Lee et al. 2000; Veal et al. 2001). Several positional candidate genes are located within the PSORS1 susceptibility locus for psoriasis: HLA-C (the leukocyte antigen C), HCR (the α-helix-coiled-coil-rod homologue), POU5F1 (the octamer transcription factor 3), TCF19 (the cell growth-regulated gene), the corneodesmosin gene, a gene encoding a plectin-like protein and three genes displaying no homology to any known sequences in any DNA database.

Veal et al. performed a SNP-haplotype-based association analysis of PSORS1 to refine the susceptibility locus (Veal et al. 2002). They identified a 10-kb major region for susceptibility for psoriasis. They showed that this restricted region comprised two biallelic markers, SNPs n.7 and n.9, with probability values clearly exceeding any other markers studied before. This 10-kb region did not contain any known gene. In addition, database analysis of this restricted region did not allow the identification of any expressed gene, although a non-expressed pseudogene was identified. Since SNPs n.7 and n.9 lie in a non coding region, respectively 7 and 4 kb centromeric to HLA-C, Veal et al. concluded that SNPs n.7 and n.9 may lie within a regulatory region influencing expression of HLA-C.

3. The Ubiquitin-Proteasome Pathway

The ubiquitin proteasome pathway has a central role in the selective degradation of intracellular proteins. Among the key proteins modulated by the proteasome are those involved in the control of inflammatory processes, cell cycle regulation, cell growth and gene expression. The proteasome is a large multimeric protease present in all eukaryotic cells that exhibits a highly conserved 20S core structure. Proteasomes are responsible for the degradation of protein substrates after they have been “tagged” by a poly-ubiquitin chain. Among others, the proteasome is known to be responsible for the degradation of IκB (Regnier et al. 1997. Cell. 90:373-383). Thus proteasome inhibition inhibits NFκB activation by blocking the degradation of its inhibitory protein IκB, and inhibition of the proteasome has been proposed as a potential mean to treat T cell-mediated disorders such as psoriasis (Zoliner et al. 2002 J Clin Invest 109:671-9).

The selective degradation of proteins through the ubiquitin proteasome pathway involves the activation of a signaling cascade that generates the covalent attachment of a polyubiquitin chain to protein targets. The polyubiquitin chain formed through the addition of multiple ubiquitin molecules to the target acts as a signal for degradation by the proteasome, a large multimeric protein complex. Ubiquitin conjugation requires the presence of three key enzymes: the ubiquitin-activating enzyme E1, the ubiquitin-conjugating enzyme E2 and the ubiquitin ligase E3.

De-ubiquitinating activities can promote the accumulation of ubiquitin in a given cell and are also thought to counteract the effects of E2/E3-mediated conjugation by removing the polyubiquitin chain from conjugated proteins prior to their degradation by the proteasome. This might either represent a means of preventing degradation by the proteasome, or might be part of those ubiquitination processes not aimed at directing protein degradation. De-ubiquitinating enzymes can be subdivided into two broad groups: ubiquitin C-terminal hydrolases (UCHs) and ubiquitin isopeptidases (UBPs) (Wilkinson, 1997). As far as UBPs are concerned, a number of published reports indicate that certain UBPs have highly specific functions. IsoT, a member of this family that has been studied in detail, is able to cleave both linear and isopeptide-linked ubiquitin, and it appears to require a free ubiquitin C-terminus for optimal activity (Wilkinson et al. 1995).

Naviglio et al cloned and characterized the UBP8 ubiquitin isopeptidase in 1998 (EMBO J. 17:3241-3250). The biochemical activity of UBP8 was determined, and it was shown that UBP8 can both hydrolyze ubiquitin-isopeptide bonds and cleave purified linear ubiquitin chains. Down-regulation of UBP8 generates a substantial derangement of the overall cell protein ubiquitination, thus indicating that UBP8 plays a general role in the ubiquitin pathway. Moreover, microinjection of antisense UBP8 cDNA in quiescent human cells prevents S-phase entry, and microinjection of antisense UBP8 cDNA In growing osteosarcoma cells determines instead an accumulation of cells in S-phase. Thus Naviglio et al. showed that inhibition of the cellular ubiquitin isopeptidase UBP8 has a striking effect on cell proliferation. In 2000, UBP8 was shown to associate with Hrs-binding protein both in vito and in cultured cells (Kato et al. J Biol. Chem. 275:37481-37487). Hrs-binding protein together with Hrs plays a regulatory role in endocytic trafficking of growth factor-receptor complexes through early endosomes. Kato et al. hypothesized that UBP8 associated with Hbp plays a positive regulatory role in proteasomal and/or lysosomal degradation of growth factor receptors.

Accordingly, proteins of the ubiquitin-proteasome pathway have been shown to play an important role in, e.g., cell cycle regulation, regulation of cell proliferation and degradation of proteins involved in Inflammation. Consequently, modulation of proteins of the ubiquitin-proteasome pathway is a treatment option for cancer and chronic Inflammatory diseases such as, e.g., rheumatoid arthritis, asthma, inflammatory bowel disease, multiple sclerosis and psoriasis.

### SUMMARY OF THE INVENTION

The present invention stems from the finding of an expressed gene located at human chromosome 6p21, within the 10-kb region that defines the major susceptibility locus for psoriasis. This gene, the UBP8rp gene, encodes a protein of the ubiquitin proteasome pathway. The UBP8rp gene comprises two introns located at nucleotide positions 1018 to 1046 of SEQ ID NO: 1 and 1676 to 1718 of SEQ ID NO: 1. Four different splice variants transcribed from the UBP8rp gene, corresponding to SEQ ID Nos. 2, 53, 55 and 56, have been isolated. The present invention is further based on the finding that the UBP8rp gene is a highly polymorphic gene. Ninety-six UBP8rp-related biallelic markers, referred to as biallelic markers 1 to 96, are disclosed in the frame of the present invention

Therefore, a first aspect of the present invention relates to an isolated gene comprising introns having a sequence of (i) nucleotides 1018 to 1046 of SEQ ID NO: 1; and (ii) nucleotides 1676 to 1718 of SEQ ID NO: 1.

The present invention further relates to an Isolated UBP8rp polynucleotide complementary to a messenger RNA transcribed from the UBP8rp gene.

The present invention is further directed to a UBP8rp polynucleotide selected from the group consisting of:
a) a polynucleotide comprising SEQ ID NO: 2;
b) a polynucleotide comprising SEQ ID NO: 52;
c) a polynucleotide comprising SEQ ID NO: 54;
d) a polynucleotide comprising SEQ ID NO: 55;
e) an allelic variant of any of (a) to (d) comprising at least one polymorphic variation compared to any of (a) to (d) respectively, wherein said polymorphic variation is selected from the group consisting of UBP8rp-related biallelic markers Nos. 1 to 96.
f) a polynucleotide complementary to any of (a) to (e).

The present invention further pertains to an isolated UBP8rp polypeptide encoded by the UBP8rp gene or by a UBP8rp polynucleotide.

The present invention is further directed to an expression vector comprising the UBP8rp gene or a UBP8rp polynucleotide.

A host cell comprising the above expression vector is a further aspect of the present invention.

The present invention is further directed to a method of making a UBP8rp polypeptide, said method comprising the steps of culturing a host cell according to the invention under conditions suitable for the production of a UBP8rp polypeptide within said host cell.

A further aspect of the invention relates to an antibody that specifically binds to a UBP8rp polypeptide.

The use of a UBP8rp polypeptide as a target for screening for natural binding partners, the use of a UBP8rp polypeptide as a target for screening candidate modulators, and the use of a modulator of a UBP8rp polypeptide for preparing a medicament for the treatment of a chronic inflammatory disease are also within the present invention.

Further, the present invention pertains to a method of assessing the efficiency of a modulator of a UBP8rp polypeptide for the treatment of psoriasis, said method comprising administering said modulator to an animal model for psoriasis; wherein a determination that said modulator ameliorates a representative characteristic of psoriasis in said animal model indicates that said modulator is a drug for the treatment of psoriasis.

In a further aspect, the present invention is directed to the use of at least one UBP8rp-related biallelic marker selected from the group consisting of biallelic markers Nos. 1, 2, 4, 6, 7, 10, 12-19, 21-30, 31-35 and 37-96 for determining whether there is a significant association between said biallelic marker and a chronic inflammatory disease.

The present invention further relates to the use of at least one UBP8rp-related biallelic marker selected from the group consisting of biallelic markers Nos. 1, 2, 4, 6, 7, 10, 12-19, 21-30, 31-35 and 37-96 for diagnosing whether an individual suffers from or is at risk of suffering from a chronic inflammatory disease.

The invention also concerns a method of genotyping comprising the steps of: (a) isolating a nucleic acid from a biological sample; and (b) detecting the nucleotide present at one or more of the UBP8rp-related biallelic markers selected from the group consisting of biallelic markers Nos. 1, 2, 4, 6, 7, 10, 12-19, 21-30, 31-35 and 37-96.

### BRIEF DESCRIPTION OF THE FIGURES

FIGS. 1A to 1G show the annotation of the gene encoding UBP8rp.

FIG. 2 shows an alignment between UBP8rp (SEQ ID NO: 3) and UBP8 (SEQ ID NO: 4).

FIG. 3 shows the rhodanese domain of UBP8rp (SEQ ID NO: 3)

FIG. 4 shows the expression levels of UBP8rp, UBP8, Cytokeratin 15 and psoriasin as determined in Example 2. The expression levels were measured in normal keratinocytes under development, and the values normalized by Day 3 and control gene mean.

FIG. 5 is a scheme of the structure of the three splice variants encoded by allele A9 of UBP8rp.

FIG. 6 shows an alignment between the UBP8rp protein encoded by allele A3 (SEQ ID NO: 3) and two splice variants encoded by alleles A9 (SEQ ID Nos. 53 and 56).

### BRIEF DESCRIPTION OF THE SEQUENCES OF THE SEQUENCE LISTING

SEQ ID NO: 1 corresponds to the genomic region comprising the UBP8rp gene (allele A3).

SEQ ID NO: 2 corresponds to the CDS coding for UBP8rp (allele A3).

SEQ ID NO: 3 corresponds to the protein sequence of UBP8rp encoded by allele A3.

SEQ ID NO: 4 corresponds to the protein sequence of UBP8.

SEQ ID Nos. 5-51 and 58-79 correspond to primers.

SEQ ID Nos. 52, 54 and 55 correspond to the messenger RNAs of three splice variants transcribed from allele A9.

SEQ ID NO: 53 corresponds to the protein encoded by SEQ ID NO: 52.

SEQ ID NO: 56 corresponds to the protein encoded by SEQ ID NO: 55.

SEQ ID NO: 57 corresponds to the peptide used to generate anti-UBP8rp antibodies.

### DETAILED DESCRIPTION OF THE INVENTION

The present invention stems from the finding of an expressed gene located at human chromosome 6p21, within the 10-kb region that defines the major susceptibility locus for psoriasis. This gene codes for a novel protein of the ubiquitin-proteasome pathway, UBP8rp. It has further been shown that one specific allele of the UBP8rp gene, allele A9, is more frequent in individuals suffering from psoriasis that in individuals not suffering from psoriasis. In addition, the expression level of the UBP8rp gene was studied by Quantitative PCR, and immunologic analysis were carried out to determine the expression profile of UBP8rp. Specifically, it has been shown that UBP8rp is modulated during growth and development of normal human keratinocytes in culture, and that UBP8rp protein is present in human skin and whole blood cells. Taken together, these results suggest that Specific variant of UBP8rp may confer abnormal proliferation capacities to psoriatic keratinocytes.

Accordingly, the present invention provides novel UBP8rp polypeptides and means to identify compounds useful in the treatment of psoriasis and other chronic inflammatory diseases such as, e.g., psoriatic arthritis, rheumatoid arthritis, asthma, inflammatory bowel disease and multiple sclerosis. Specifically, the invention relates to the use of UBP8rp polypeptides as targets for screening for modulators thereof. The use of said modulators for treating psoriasis and other chronic inflammatory diseases, and the use of novel biallelic markers located in the UBP8rp gene for diagnosing psoriasis and other chronic inflammatory diseases are further aspects of the present invention.

1. Polynucleotides of the Present Invention

A first aspect of the present invention relates to an isolated gene comprising introns having a sequence of (i) nucleotides 1018 to 1046 of SEQ ID NO: 1; and (ii) nucleotides 1676 to 1718 of SEQ ID NO: 1.

As used herein, the term “intron” refers to a sequence of nucleotides interrupting the protein-coding sequences of a gene. Introns are transcribed into primary RNA but are cut out of the primary RNA to generate a messenger RNA that it is translated into protein.

As used herein, the term “gene” refers to a sequence of nucleotides located in a particular position on a particular chromosome that encodes a specific protein. A gene usually comprises exons, introns, 5′ and 3′ untranslated regions, and upstream and downstream regulatory sequences. A gene may encode different isoforms of the same protein. These isoforms may be generated by, e.g., alternative splicing events or start of translation from alternative initiation codons. The term “gene”, as used herein, does not include pseudogenes.

As further used in this specification, the term “UBP8-rp gene” refers to the gene comprising the introns shown at nucleotides 1018 to 1046 and nucleotides 1676 to 1718 of SEQ ID NO: 1. This gene is located at locus 6p21, within the major susceptibility locus for psoriasis, and codes for the UBP8rp protein. The term “UBP8-rp gene” encompasses all naturally occurring alleles of such a gene. Specifically, the term “UBP8-rp gene” encompasses all alleles comprising at least 1, 2, 3, 4, 5, 10, 15 or 20 UBP8rp-related biallelic markers. Specifically, the term “UBP8-rp gene” encompasses an allelic variant wherein at least one intron of the UBP8rp gene comprises at least one polymorphic variation, wherein said polymorphic variation is selected from the group consisting of UBP8rp-related biallelic markers Nos. 12, 49, 69 and 70.

Furthermore, procedures known in the art can be used to obtain other allelic variants of the UBP8rp gene using information from the sequences disclosed herein. For example, other allelic variants may be isolated and identified by making suitable probes or primers from the sequences provided herein and screening a suitable nucleic acid source for allelic variants using any technique known to those skilled in the art.

The terms “comprising”, “consisting of”, or “consisting essentially of” have distinct meanings. However, each term may be substituted for another herein to change the scope of the invention.

Another aspect of the present invention relates to an isolated polynucleotide complementary to a messenger RNA transcribed from the gene of claim 1.

As used herein, the term ‘messenger RNA’ (mRNA) refers to the processed RNA molecule that does not comprise any intron sequence. The term messenger RNA encompasses all alternative splice variants translated from the UBP8rp gene.

Such a messenger RNA may comprise any combination of exon of the UBP8rp gene. In one embodiment, the UBP8rp polynucleotide comprises exon 1 comprising nucleotides 1 to 167 of SEQ ID NO: 2 or an allelic variant thereof. In another embodiment, the UBP8rp polynucleotide comprises exon 2 comprising nucleotides 168 to 796 of SEQ ID NO: 2 or an allelic variant thereof. In still another embodiment, the UBP8rp polynucleotide comprises exon 3 comprising either nucleotides 797 to 1449 of SEQ ID NO: 2, or nucleotides 797 to 1458 of SEQ ID NO: 52, or an allelic variant thereof. In still another embodiment, the UBP8rp polynucleotide comprises exon 3A comprising nucleotides 797 to 1035 of SEQ ID NO: 55 or an allelic variant thereof. Preferably, the UBP8rp polynucleotide comprises SEQ ID NO: 2, SEQ ID NO: 52, SEQ ID NO: 55 or an allelic variant thereof. Preferably, the allelic variants comprise at least one polymorphic variation selected from the group consisting of UBP8rp-related biallelic markers Nos. 1 to 96. More preferably, the at least one polymorphic variation is located within a coding sequence. Most preferably, the at least one polymorphic variation leads to a change of the sequence of the encoded polypeptide.

Another aspect of the present invention relates to a polynucleotide selected from the group consisting of:
a) a polynucleotide comprising SEQ ID NO: 2;
b) a polynucleotide comprising SEQ ID NO: 52;
c) a polynucleotide comprising SEQ ID NO: 54;
d) a polynucleotide comprising SEQ ID NO: 55;
e) an allelic variant of any of (a) to (d) comprising at least one polymorphic variation compared to any of (a) to (d) respectively, wherein said polymorphic variation is selected from the group consisting of UBP8rp-related biallelic markers Nos. 1 to 96 shown below.

f) a polynucleotide complementary to any of (a) to (e).

| Biallelic | Position on | Alternative |
|---|---|---|
| marker No. | SEQ ID NO: 1 | nucleotides |
| 1 | 1199 | A/G |
| 2 | 1262 | C/T |
| 3 | 1426 | C/G |
| 4 | 1444 | G/T |
| 5 | 1487 | A/G |
| 6 | 1490 | A/G |
| 7 | 1505 | G/T |
| 8 | 1518 | C/T |
| 9 | 1554 | C/T |
| 10 | 1630 | A/G |
| 11 | 1638 | A/T |
| 12 | 1680 | A/G |
| 13 | 1895 | A/G |
| 14 | 2180 | A/G |
| 15 | 2449 | A/T |
| 16 | 2721 | G/T |
| 17 | 3127 | A/G |
| 18 | 3137 | C/T |
| 19 | 3138 | A/G |
| 20 | 3183 | A/G |
| 21 | 3222 | C/G |
| 22 | 3269 | C/T |
| 23 | 3445 | C/T |
| 24 | 3470 | A/G |
| 25 | 3915 | C/T |
| 26 | 3973 | A/C |
| 27 | 4254 | A/G |
| 28 | 4472 | A/T |
| 29 | 4660 | C/T |
| 30 | 4770 | A/G |
| 31 | 4919 | A/G |
| 32 | 4973 | C/T |
| 33 | 5063 | C/T |
| 34 | 5065 | G/T |
| 35 | 5079 | C/T |
| 36 | 5080 | C/T |
| 37 | 5088 | C/G |
| 38 | 5090 | C/T |
| 39 | 5407 | C/T |
| 40 | 5466 | A/G |
| 41 | 5520 | C/T |
| 42 | 829 | A/G |
| 43 | 856 | A/G |
| 44 | 902 | insertion of G |
| 45 | 908 | insertion of A |
| 46 | 972 | A/G |
| 47 | 975 | A/G |
| 48 | 1006 | C/T |
| 49 | 1018 | A/G |
| 50 | 1048 | A/C |
| 51 | 1056 | C/T |
| 52 | 1069 | G/T |
| 53 | 1073 | A/G |
| 54 | 1079 | A/G |
| 55 | 1108 | A/G |
| 56 | 1154 | A/G |
| 57 | 1181 | A/G |
| 58 | 1236 | A/G |
| 59 | 1263 | A/G |
| 60 | 1274 | A/G |
| 61 | 1319 | G/T |
| 62 | 1334 | A/G |
| 63 | 1444 | G/T |
| 64 | 1466 | C/T |
| 65 | 1489 | A/G |
| 66 | 1508 | C/T |
| 67 | 1521 | G/T |
| 68 | 1543 | A/G |
| 69 | 1687 | A/C |
| 70 | 1707 | deletion of C |
| 71 | 1728 | A/G |
| 72 | 1742 | C/T |
| 73 | 1810 | C/T |
| 74 | 1813 | A/C |
| 75 | 1841 | C/T |
| 76 | 1874 | C/G |
| 77 | 1875 | A/G |
| 78 | 1890 | A/C |
| 79 | 1907 | A/G |
| 80 | 1909 | C/T |
| 81 | 1921 | A/C |
| 82 | 1922 | A/G |
| 83 | 1957 | A/G |
| 84 | 1959 | A/G |
| 85 | 1976 | C/T |
| 86 | 1992 | C/T |
| 87 | 1993 | C/T |
| 88 | 2096 | C/G |
| 89 | 2135 | A/G |
| 90 | 2192 | A/G |
| 91 | 2230 | C/G |
| 92 | 2275 | C/T |
| 93 | 2314 | A/G |
| 94 | 2370 | A/C |
| 95 | 2375 | A/T |
| 96 | 2525 | C/T |

As further used herein, the term “UBP8rp polynucleotide” refers to an isolated polynucleotide complementary to a messenger RNA transcribed from the UBP8rp gene, to a polynucleotide of any of (a) to (f) of the above paragraph, or to a fragment thereof.

The fragment of a UBP8rp polynucleotides may be at least 100, 200, 300, 400, 500, 600, 700, 800, 900, 1000, 1100, 1200, 1300, 1400, 1500, 1600, 1700, 1800, 1900 or 2000 nucleotides in length.

Any procedures known in the art can be used to obtain UBP8rp polynucleotides. UBP8rp polynucleotides can for example be obtained as described in Example 1.

The present invention also encompasses UBP8rp polynucleotides for use as primers and probes. Such primers are useful in order to detect the presence of at least a copy of a UBP8rp polynucleotide, complement, or variant thereof in a test sample. The probes of the present invention are useful for a number of purposes. They can preferably be used in Southern hybridization to genomic DNA. The probes can also be used to detect PCR amplification products. They may also be used to detect mismatches in the UBP8rp using other techniques. They may further be used for in situ hybridization. Preferred primers of the present invention are those of SEQ ID Nos. 5-51.

The present invention also encompasses polynucleotides UBP8rp polynucleotide that codes for a fragment of a UBP8rp polypeptide. The fragment may for example consist of an antigenic epitope of the UBP8rp and find use in production of antibodies.

Any of the polynucleotides, primers and probes of the present invention can be conveniently immobilized on a solid substrate, such as, e.g., a microarray. A substrate comprising a plurality of oligonucleotide primers or probes of the invention may be used either for detecting or amplifying targeted sequences in the UBP8rp gene, may be used for detecting mutations in the coding or in the non-coding sequences of the UBP8rp mRNAs, and may also be used to determine expression of UBP8rp mRNAs in different contexts such as in different tissues, at different stages of a process (embryo development, disease treatment), and In patients versus healthy individuals.

2. Polypeptides of the Present Invention

Another aspect of the present invention relates to a purified polypeptide encoded by the UBP8rp gene or by a UBP8rp polynucleotide.

In a preferred embodiment, the UBP8rp polypeptide is selected from the group consisting of.
a) a polypeptide comprising SEQ ID NO:3;
b) a polypeptide comprising a span of at least 470 amino acids of SEQ ID NO: 3;
c) a polypeptide comprising a span of at least 15 amino acids of SEQ ID NO: 3, wherein said span falls within amino acids 467 to 482 of SEQ ID NO: 3;
d) an allelic variant of any of (a) to (c) comprising at least one polymorphic variation compared to any of (a) to (c) respectively, wherein said polymorphic variation is encoded by a codon comprising a UBP8rp-related biallelic marker selected from selected from the group consisting of UBP8rp-related biallelic markers Nos. 1 to 96;
e) a mutein of any of (a) to (c), wherein the amino acid sequence has at least 95%, 96%, 97%, 98% or 99% identity to at least one of the sequences in (a) to (c);
f) a mutein of any of (a) to (c) which is encoded by a nucleic acid which hybridizes to the complement of a DNA sequence encoding any of (a) to (c) under highly stringent conditions; and
g) a mutein of any of (a) to (c) wherein any changes in the amino acid sequence are conservative amino acid substitutions of the amino acid sequences in (a) to (c).

In another preferred embodiment, the UBP8rp polypeptide is selected from the group consisting of:
a) a polypeptide comprising SEQ ID NO:53;
b) a polypeptide comprising a span of at least 470 amino acids of SEQ ID NO: 53;
c) a polypeptide comprising a span of at least 15 amino acids of SEQ ID NO: 53, wherein said span falls within amino acids 467 to 485 of SEQ ID NO: 53;
d) a mutein of any of (a) to (c), wherein the amino acid sequence has at least 95%, 96%, 97%, 98% or 99% identity to at least one of the sequences in (a) to (c);
e) a mutein of any of (a) to (c) which is encoded by a nucleic add which hybridizes to the complement of a DNA sequence encoding any of (a) to (c) under highly stringent conditions; and
f) a mutein of any of (a) to (c) wherein any changes in the amino acid sequence are conservative amino acid substitutions of the amino acid sequences in (a) to (c).

In still another preferred embodiment, the UBP8rp polypeptide is selected from the group consisting of:
a) a polypeptide comprising SEQ ID NO:56;
b) a polypeptide comprising a span of at least 270 amino acids of SEQ ID NO: 56;
c) a polypeptide comprising a span of at least 15 amino acids of SEQ ID NO: 56, wherein said span comprises amino acids 266 and 267 of SEQ ID NO: 56;
d) a mutein of any of (a) to (c), wherein the amino acid sequence has at least 95%, 96%, 97%, 98% or 99% identity to at least one of the sequences in (a) to (c);
e) a mutein of any of (a) to (c) which is encoded by a nucleic acid which hybridizes to the complement of a DNA sequence encoding any of (a) to (c) under highly stringent conditions; and
f) a mutein of any of (a) to (c) wherein any changes in the amino acid sequence are conservative amino acid substitutions of the amino acid sequences in (a) to (c).

The term “UBP8rp polypeptide” is used herein to embrace all of the polypeptides of the present invention.

In a preferred embodiment, the UBP8rp polypeptide corresponds to a full-length UBP8rp protein. The UBP8rp protein is a member of the ubiquitin proteasome pathway, as described in Example 1. UBP8rp plays a role in the ubiquitin-conjugation and de-ubiquitination of intracellular proteins, either by de-ubiquitinating said intracellular proteins, or by regulating ubiquitinating and de-ubiquitinating enzymes. The biological activity of a UBP8rp polypeptide refers to the modulation of the ubiquitination state of intracellular proteins by UBP8rp.

The present invention is also directed to polypeptides consisting of a fragment of at least 10, 15, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470 or 480 amino acids of SEQ ID Nos. 3, 53 or 56. Preferably, said fragment falls within amino acids 467 to 482 of SEQ ID NO: 3, or within amino acids 467 to 485 of SEQ ID NO: 53, or comprises amino acids 266 and 267 of SEQ ID NO: 56.

The present invention is also directed to naturally occurring, recombinant, or chimeric polypeptides comprising any of the above fragments.

One embodiment is directed to a UBP8rp polypeptide wherein the amino acid at position 107 of SEQ ID NO: 3 is an arginine. Another embodiment is directed to a UBP8rp polypeptide wherein the amino acid at position 107 of SEQ ID NO: 3 is a lysine.

One embodiment is directed to a UBP8rp polypeptide wherein the amino acid at position 128 of SEQ ID NO: 3 is an threonine. Another embodiment is directed to a UBP8rp polypeptide wherein the amino acid at position 128 of SEQ ID NO: 3 is a methionine.

One embodiment is directed to a UBP8rp polypeptide wherein the amino acid at position 183 of SEQ ID NO: 3 is an asparagine. Another embodiment is directed to a UBP8rp polypeptide wherein the amino acid at position 183 of SEQ ID NO: 3 is an histidine.

One embodiment is directed to a UBP8rp polypeptide wherein the amino acid at position 189 of SEQ ID NO: 3 is an asparagine. Another embodiment is directed to a UBP8rp polypeptide wherein the amino acid at position 189 of SEQ ID NO: 3 is a tyrosine.

One embodiment is directed to a UBP8rp polypeptide wherein the amino acid at position 203 of SEQ ID NO: 3 is glycine. Another embodiment is directed to a UBP8rp polypeptide wherein the amino acid at position 203 of SEQ ID NO: 3 is a glutamic acid.

One embodiment is directed to a UBP8rp polypeptide wherein the amino acid at position 204 of SEQ ID NO: 3 is an arginine. Another embodiment is directed to a UBP8rp polypeptide wherein the amino acid at position 204 of SEQ ID NO: 3 is a lysine.

One embodiment is directed to a UBP8rp polypeptide wherein the amino acid at position 209 of SEQ ID NO: 3 is a glycine. Another embodiment is directed to a UBP8rp polypeptide wherein the amino acid at position 209 of SEQ ID NO: 3 is a valine.

One embodiment is directed to a UBP8rp polypeptide wherein the amino acid at position 251 of SEQ ID NO: 3 is an glycine. Another embodiment is directed to a UBP8rp polypeptide wherein the amino acid at position 251 of SEQ ID NO: 3 is an arginine.

One embodiment is directed to a UBP8rp polypeptide wherein the amino acid at position 325 of SEQ ID NO: 3 is an glutamic acid. Another embodiment is directed to a UBP8rp polypeptide wherein the amino acid at position 325 of SEQ ID NO: 3 is a lysine.

One embodiment is directed to a UBP8rp polypeptide wherein the amino acid at position 420 of SEQ ID NO: 3 is an alanine. Another embodiment is directed to a UBP8rp polypeptide wherein the amino acid at position 420 of SEQ ID NO: 3 is a threonine.

Further embodiments are directed to allelic variants of a polypeptide of SEQ ID NO: 3 or fragments thereof. Preferred allelic variants are those comprising at least UBP8rp biallelic markers of the present invention.

Further embodiments are directed to muteins. As used herein the term “muteins” refers to analogs of UBP8rp, in which one or more of the amino acid residues of a natural UBP8rp are replaced by different amino acid residues, or are deleted, or one or more amino acid residues are added to the natural sequence of UBP8rp, without lowering considerably the activity of the resulting products as compared with the wild-type UBP8rp. These muteins are prepared by known synthesis and/or by site-directed mutagenesis techniques, or any other known technique suitable therefore.

Muteins of UBP8rp, which can be used in accordance with the present invention, or nucleic acid coding thereof, include a finite set of substantially corresponding sequences as substitution peptides or polynucleotides which can be routinely obtained by one of ordinary skill in the art, without undue experimentation, based on the teachings and guidance presented herein.

UBP8rp polypeptides in accordance with the present invention include proteins encoded by a nucleic acid, such as DNA or RNA, which hybridizes to DNA or RNA, which encodes UBP8RPb, in accordance with the present invention, under moderately or highly stringent conditions. The term “stringent conditions” refers to hybridization and subsequent washing conditions, which those of ordinary skill in the art conventionally refer to as “stringent”. See Ausubel et al., Current Protocols in Molecular Biology, supra, Interscience, N.Y., §6.3 and 6.4 (1987, 1992), and Sambrook et al. (Sambrook, J. C., Fritsch, E. F., and Maniatis, T. (1989) Molecular Cloning: A Laboratory Manual, Cold Spring Harbor Laboratory Press, Cold Spring Harbor, N.Y.).

Without limitation, examples of stringent conditions include washing conditions 12-20° C. below the calculated Tm of the hybrid under study in, e.g., 2×SSC and 0.5% SDS for 5 minutes, 2×SSC and 0.1% SDS for 15 minutes; 0.1×SSC and 0.5% SDS at 37° C. for 30-60 minutes and then, a 0.1×SSC and 0.5% SDS at 68° C. for 30-60 minutes. Those of ordinary skill in this art understand that stringency conditions also depend on the length of the DNA sequences, oligonucleotide probes (such as 10-40 bases) or mixed oligonucleotide probes. If mixed probes are used, it is preferable to use tetramethyl ammonium chloride (TMAC) instead of SSC.

The polypeptides of the present invention include muteins having an amino acid sequence at least 50% identical, more preferably at least 60% identical, and still more preferably 70%, 80%, 90%, 95%, 96%, 97%, 98% or 99% identical to a UBP8RPb polypeptide of the present invention. By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% (5 of 100) of the amino acid residues in the subject sequence may be inserted, deleted, or substituted with another amino acid.

For sequences where there is not an exact correspondence, a “% identity” may be determined. In general, the two sequences to be compared are aligned to give a maximum correlation between the sequences. This may include inserting “gaps” in either one or both sequences, to enhance the degree of alignment. A % identity may be determined over the whole length of each of the sequences being compared (so-called global alignment), that is particularly suitable for sequences of the same or very similar length, or over shorter, defined lengths (so-called local alignment), that is more suitable for sequences of unequal length. Methods for comparing the identity and homology of two or more sequences are well known in the art. Thus for Instance, programs available in the Wisconsin Sequence Analysis Package, version 9.1 (Devereux J et al. (1984) Nucleic Acids Res. 12:387-395), for example the programs BESTFIT and GAP, may be used to determine the % Identity between two polynucleotides and the % identity and the % homology between two polypeptide sequences. BESTFIT uses the “local homology” algorithm of Smith and Waterman (1981, J Mol Evol. 18:3846) and finds the best single region of similarity between two sequences. Other programs for determining identity and/or similarity between sequences are also known in the art, for instance the BLAST family of programs (Altschul et al. (1990) J Mol. Biol. 215:403-410), accessible through the home page of the NCBI at world wide web site ncbi.nim.nih.gov) and FASTA (Pearson (1990) Methods in Enzymology, 183:63-99; Pearson and Lipman (1988) Proc Nat Acad Sci USA, 85:2444-2448).

Preferred changes for muteins in accordance with the present invention are what are known as “conservative” substitutions. Conservative amino acid substitutions of UBP8rp polypeptides, may include synonymous amino acids within a group which have sufficiently similar physicochemical properties that substitution between members of the group will preserve the biological function of the molecule (Grantham (1974) Science 185:862-864). It is clear that insertions and deletions of amino acids may also be made in the above-defined sequences without altering their function, particularly if the insertions or deletions only involve a few amino acids, e.g. under thirty, and preferably under ten, and do not remove or displace amino acids which are critical to a functional conformation, e.g. cysteine residues. Proteins and muteins produced by such deletions and/or Insertions come within the purview of the present invention.

Preferably, the synonymous amino acid groups are those defined in Table I. More preferably, the synonymous amino acid groups are those defined in Table II; and most preferably the synonymous amino acid groups are those defined in Table III.

TABLE I
Preferred Groups of Synonymous Amino Acids

| Amino Acid | Synonymous Group |
|---|---|
| Ser | Ser, Thr, Gly, Asn |
| Arg | Arg, Gln, Lys, Glu, His |
| Leu | Ile, Phe, Tyr, Met, Val, Leu |
| Pro | Gly, Ala, Thr, Pro |
| Thr | Pro, Ser, Ala, Gly, His, Gln, Thr |
| Ala | Gly, Thr, Pro, Ala |
| Val | Met, Tyr, Phe, Ile, Leu, Val |
| Gly | Ala, Thr, Pro, Ser, Gly |
| Ile | Met, Tyr, Phe, Val, Leu, Ile |
| Phe | Trp, Met, Tyr, Ile, Val, Leu, Phe |
| Tyr | Trp, Met, Phe, Ile, Val, Leu, Tyr |
| Cys | Ser, Thr, Cys |
| His | Glu, Lys, Gln, Thr, Arg, His |
| Gln | Glu, Lys, Asn, His, Thr, Arg, Gln |
| Asn | Gln, Asp, Ser, Asn |
| Lys | Glu, Gln, His, Arg, Lys |
| Asp | Glu, Asn, Asp |
| Glu | Asp, Lys, Asn, Gln, His, Arg, Glu |
| Met | Phe, Ile, Val, Leu, Met |
| Trp | Trp |

TABLE II
More Preferred Groups of Synonymous Amino Acids

| Amino Acid | Synonymous Group |
|---|---|
| Ser | Ser |
| Arg | His, Lys, Arg |
| Leu | Leu, Ile, Phe, Met |
| Pro | Ala, Pro |
| Thr | Thr |
| Ala | Pro, Ala |
| Val | Val, Met, Ile |
| Gly | Gly |
| Ile | Ile, Met, Phe, Val, Leu |
| Phe | Met, Tyr, Ile, Leu, Phe |
| Tyr | Phe, Tyr |
| Cys | Cys, Ser |
| His | His, Gln, Arg |
| Gln | Glu, Gln, His |
| Asn | Asp, Asn |
| Lys | Lys, Arg |
| Asp | Asp, Asn |
| Glu | Glu, Gln |
| Met | Met, Phe, Ile, Val, Leu |
| Trp | Trp |

TABLE III
Most Preferred Groups of Synonymous Amino Acids

| Amino Acid | Synonymous Group |
|---|---|
| Ser | Ser |
| Arg | Arg |
| Leu | Leu, Ile, Met |
| Pro | Pro |
| Thr | Thr |
| Ala | Ala |
| Val | Val |
| Gly | Gly |
| Ile | Ile, Met, Leu |
| Phe | Phe |
| Tyr | Tyr |
| Cys | Cys, Ser |
| His | His |
| Gln | Gln |
| Asn | Asn |
| Lys | Lys |
| Asp | Asp |
| Glu | Glu |
| Met | Met, Ile, Leu |
| Trp | Met |

Examples of production of amino acid substitutions in proteins which can be used for obtaining muteins of UBP8rp, polypeptides for use in the present invention include any known method steps, such as presented in U.S. Pat. Nos. 4,959,314, 4,588,585 and 4,737,462, to Mark et al; U.S. Pat. No. 5,116,943 to Koths et al., U.S. Pat. No. 4,965,195 to Namen et al; U.S. Pat. No. 4,879,111 to Chong et al; and U.S. Pat. No. 5,017,691 to Lee et al; and lysine substituted proteins presented in U.S. Pat. No. 4,904,584 (Shaw et al).

Preferably, the muteins of the present invention exhibit substancially the same biological activity as the UBP8RPb polypeptide to which it corresponds.

In other embodiments, UBP8rp polypeptides do not exhibit the biological activity as the UBP8RPb polypeptide to which it corresponds. Other uses of the polypeptides of the present invention include, inter alia, as epitope tags, in epitope mapping, and as molecular weight markers on SDS-PAGE gels or on molecular sieve gel filtration columns using methods known to those of skill in the art. Such polypeptides can be used to raise polyclonal and monoclonal antibodies, which are useful in assays for detecting UBP8rp expression, or for purifying UBP8rp. As a matter of example, a further specific use for UBP8rp polypeptides is the use of such polypeptides the yeast two-hybrid system to capture UBP8rp binding proteins, which are candidate modulators according to the present invention, as further detailed below.

3. Vectors, Host Cells and Host Organisms of the Present Invention

The present invention also relates to vectors comprising the UBP8rp gene or a UBP8rp polynucleotide. More particularly, the present invention relates to expression vectors which include the UBP8rp gene or a UBP8rp polynucleotide. Preferably, such expression vectors comprise a polynucleotide encoding a UBP8rp polypeptide.

The term “vector” is used herein to designate either a circular or a linear DNA or RNA compound, which is either double-stranded or single-stranded, and which comprise at least one polynucleotide of the present invention to be transferred in a cell host or in a unicellular or multicellular host organism. An “expression vector” comprises appropriate signals in the vectors, said signals including various regulatory elements, such as enhancers/promoters from both viral and mammalian sources that drive expression of the Inserted polynucleotide in host cells. Selectable markers for establishing permanent, stable cell clones expressing the products such as, e.g., a dominant drug selection, are generally included in the expression vectors of the invention, as they are elements that link expression of the drug selection markers to expression of the polypeptide. The expression vector may also comprise an amplifiable marker. This amplifiable marker may be selected from the group consisting of, e.g., adenosine deaminase (ADA), dihydrofolate reductase (DHFR), multiple drug resistance gene (MDR), ornithine decarboxylase (ODC) and N-(phosphonacetyl)-L-aspartate resistance (CAD).

Additionally, the expression vector may be a fusion vector driving the expression of a fusion polypeptide between a UBP8rp polypeptide and a heterologous polypeptide. For example, the heterologous polypeptide may be a selectable marker such as, e.g, a luminescent protein, or a polypeptide allowing the purification of the fusion polypeptide.

The polynucleotides of the present invention may be used to, e.g., express the encoded polypeptide in a host cell for producing the encoded polypeptide. The polynucleotides of the present invention may further be used to express the encoded polypeptide in a host cell for screening assays. Screening assays are of particular interest for identifying modulators and/or binding partners of UBP8rp polypeptides as further detailed be low. The polynucleotides of the present invention may also be used to express the encoded polypeptide in a host organism for producing a beneficial effect. In such procedures, the encoded protein may be transiently expressed in the host organism or stably expressed in the host organism. The encoded polypeptide may have any of the properties described herein. The encoded polypeptide may be a protein which the host organism lacks or, alternatively, the encoded protein may augment the existing levels of the protein in the host organism.

In one embodiment, the expression vector is a gene therapy vector. Viral vector systems that have application in gene therapy have been derived from, e.g., adenoviral vectors and retroviral vectors.

Another object of the Invention comprises a host cell comprising the UBP8rp gene or a UBP8rp polynucleotide. Such host cells may have been transformed, transfected or transduced with a polynucleotide encoding a UBP8rp polypeptide. Also included are host cells that are transformed, transfected or transduced with a recombinant vector such as one of those described above. The cell hosts of the present invention can comprise any of the polynucleotides of the present invention.

Any host cell known by one of skill in the art may be used. Preferred host cells used as recipients for the polynucleotides and expression vectors of the invention include:

a) Prokaryotic host cells: Escherichia coli strains (I.E.DH5-α strain), Bacillus subtilis, Salmonella typhimurium, and strains from species like Pseudomonas, Streptomyces and Staphylococcus.

b) Eukaryotic host cells: CHO (ATCC No. CCL-61), HeLa cells (ATCC No. CCL2; No. CCL2.1; No. CCL2.2), Cv 1 cells (ATCC No. CCL70), COS cells (ATCC No. CRL1650; No. CRL1651), Sf-9 cells (ATCC No. CRL1711), C127 cells (ATCC No. CRL-1804), 3T3 (ATCC No. CRL-6361), human kidney 293. (ATCC No. 45504; No. CRL-1573), BHK (ECACC No. 84100501; No. 84111301), Saccharomyces cerevisiae strains such as AH109 and Y184, and Aspergillus niger strains.

Another object of the invention comprises methods of making the above vectors and host cells by recombinant techniques. Any well-known technique for constructing an expression vector and for delivering it to a cell may be used for construction and delivering the vectors of the present invention. Such techniques include but are not limited to the techniques detailed in the examples.

Another object of the present invention is a transgenic animal which includes within a plurality of its cells a cloned recombinant UBP8rp polynucleotide. The terms “transgenic animals” or “host animals” are used herein to designate animals that have their genome genetically and artificially manipulated so as to include one of the nucleic acids according to the invention. The cells affected may be somatic, germ cells, or both. Preferred animals are non-human mammals and include those belonging to a genus selected from Mus (e.g. mice), Rattus (e.g. rats) and Oryctogalus (e.g. rabbits) which have their genome artificially and genetically altered by the insertion of a nucleic acid according to the invention. In one embodiment, the invention encompasses non-human host mammals and animals comprising a recombinant vector of the invention or a UBP8rp polynucleotide disrupted by homologous recombination with a knock out vector.

In a preferred embodiment, these transgenic animals may be good experimental models in order to study diverse pathologies related to UBP8rp function. In particular, a transgenic animal wherein (i) an antisense mRNA binding to naturally occurring UBP8rp mRNAs is transcribed; or (ii) an mRNA expressing a UBP8rp polypeptide; may be a good animal model for psoriasis and/or other chronic inflammatory diseases.

4. Methods of Making the Polypeptides of the Present Invention

The present invention also relates to methods of making a UBP8rp polypeptide.

In one embodiment, the UBP8rp polypeptides of the present invention are isolated from natural sources, including tissues and cells, whether directly isolated or cultured cells, of humans or non-human animals. Soluble forms of UBP8rp may be isolated from body fluids. Methods for extracting and purifying natural membrane spanning proteins are known in the art, and include the use of detergents or chaotropic agents to disrupt particles followed by, e.g., differential extraction and separation of the polypeptides by ion exchange chromatography, affinity chromatography, sedimentation according to density, and gel electrophoresis. The method described in Example 4 may for example be used. Polypeptides of the Invention also can be purified from natural sources using antibodies directed against the polypeptides of the invention, such as those described herein, in methods which are well known in the art of protein purification.

In a preferred embodiment, the UBP8rp polypeptides of the invention are recombinantly produced using routine expression methods known in the art. The polynucleotide encoding the desired polypeptide is operably linked to a promoter into an expression vector suitable for any convenient host. Both eukaryotic and prokaryotic host systems may be used in forming recombinant polypeptides. The polypeptide is then isolated from lysed cells or, if a soluble form is produced, from the culture medium and purified to the extent needed for its intended use.

Consequently, a further embodiment of the present invention is a method of making a polypeptide of the present invention, said method comprising the steps of:

a) obtaining a polynucleotide encoding a UBP8rp polypeptide;

b) inserting said polynucleotide in an expression vector such that the polynucleotide is operably linked to a promoter; and

introducing said expression vector into a host cell whereby said host cell produces said polypeptide.

In a preferred embodiment, the method further comprises the step of isolating the polypeptide. The skilled person will appreciate that any step of this method may be carried out separately. The product of each step may be transferred to another step in order to carry out the subsequent step.

In further embodiments, said polynucleotide consists of a coding sequence. In another aspect of this embodiment, said polynucleotide is a polynucleotide comprising SEQ ID NO: 2 or a fragment thereof.

A further aspect of the Invention relates to a method of making a polypeptide, said method comprising the steps of culturing a host cell comprising an expression vector comprising a UBP8rp polynucleotide under conditions suitable for the production of a UBP8rp polypeptide within said host cell. In a preferred embodiment, the method further comprises the step of purifying said polypeptide from the culture.

In another embodiment, it is often advantageous to add to the recombinant polynucleotide encoding a UBP8rp polypeptide additional nucleotide sequence which codes for secretory or leader sequences, pro-sequences, sequences which aid in purification, such as multiple histidine residues or GST tags, or an additional sequence for stability during recombinant production. Soluble portions of the UBP8rp polypeptide may be, e.g., linked to an Ig-Fc part in order to generate stable soluble variants.

A polypeptide of this invention can be recovered and purified from recombinant cell cultures by well-known methods including but not limited to differential extraction, ammonium sulfate or ethanol precipitation, acid extraction, anion or cation exchange chromatography, high performance liquid chromatography, phosphocellulose chromatography, hydrophobic interaction chromatography, affinity chromatography, hydroxylapatite chromatography, immunochromatography and lectin chromatography.

The expressed UBP8rp polypeptide may be purified using any standard immunochromatography techniques. In such procedures, a solution containing the polypeptide of interest, such as the culture medium or a cell extract, is applied to a column having antibodies against the polypeptide attached to the chromatography matrix. The recombinant protein is allowed to bind the immunochromatography column. Thereafter, the column is washed to remove non-specifically bound proteins. The specifically bound secreted protein is then released from the column and recovered using standard techniques.

The purified UBP8rp polypeptide obtained by any of these methods may further be formulated into a pharmaceutical composition.

5. Antibodies of the Present Invention

The present invention further relates to antibodies that specifically bind to the UBP8rp polypeptides of the present invention. As further used herein, such an antibody is referred to as an “anti-UBP8rp antibody”. More specifically, the antibodies bind to the epitopes of the polypeptides of the present invention. The antibodies of the present invention include IgG (including IgG1, IgG2, IgG3, and IgG4), IgA (including IgA1 and IgA2), IgD, IgE, or IgM, and IgY. The term “antibody” (Ab) refers to a polypeptide or group of polypeptides which are comprised of at least one binding domain, where a binding domain is formed from the folding of variable domains of an antibody compound to form three-dimensional binding spaces with an internal surface shape and charge distribution complementary to the features of an antigenic determinant of an antigen, which allows an immunological reaction with the antigen. As used herein, the term “antibody” is meant to include whole antibodies, including single-chain whole antibodies, and antigen binding fragments thereof. In a preferred embodiment the antibodies are human antigen binding antibody fragments of the present invention include, but are not limited to, Fab, Fab′ F(ab)2 and F(ab′)2, Fd, single-chain Fvs (scFv), single-chain antibodies, disulfide-linked Fvs (sdFv) and fragments comprising either a V_{L} or V_{H} domain. The antibodies may be from any animal origin including birds and mammals. Preferably, the antibodies are from human, mouse, rabbit, goat, guinea pig, camel, horse or chicken. The present invention further includes humanized monoclonal and polyclonal antibodies, which specifically bind the polypeptides of the present invention.

In one embodiment, the anti-UBP8rp antibody specifically binds to all allelic variants of the UBP8rp polypeptide. Preferably, such an antibody recognizes an epitope that does not comprise any UBP8rp-related biallelic marker. Preferred such antibodies include those recognizing an epitope that is located within a region that is common to the polypeptides of SEQ ID Nos. 3, 53 and 56.

In another embodiment, the anti-UBP8rp antibody specifically binds to a given allelic variant of the UBP8rp polypeptide.

In another embodiment, the anti-UBP8rp antibody specifically binds to allelic variants of the UBP8rp polypeptide comprising a given polymorphic variation.

Preferred antibodies of the present invention recognize an epitope comprising at least one amino acid within amino acids 467 to 482 of SEQ ID NO: 3, wherein said one or more amino-acids are required for binding of the antibody to a UBP8rp polypeptide.

Preferred antibodies of the present invention recognize an epitope comprising at least one amino acid within amino acids 483 to 485 of SEQ ID NO: 52, wherein said at least one amino-acid is required for binding of the antibody to a UBP8rp polypeptide.

A preferred antibody of the present invention is an antibody generated using the immunogenic polypeptide of SEQ ID NO: 57.

A preferred embodiment of the invention is a method of specifically binding an antibody of the present invention to a UBP8rp polypeptide. This method comprises the step of contacting the antibody of the present invention with a UBP8rp polypeptide under conditions in which said antibody can specifically bind to said polypeptide. Such conditions are well known to those skilled in the art. This method may be used to, e.g., detect, purify, or activate or inhibit the activity of UBP8rp polypeptides.

The invention further relates to antibodies that act as modulators of the polypeptides of the present invention. Preferred antibodies are modulators that enhance the binding activity or the biological activity of the UBP8rp polypeptide to which they bind. These antibodies may act as modulators for the biological activity of the UBP8rp polypeptide.

6. Uses of the Polypeptides of the Present Invention

The present invention is also directed to the use of a UBP8rp polypeptide as a target for screening candidate modulators.

As used herein, the term “modulator” refers to a compound that increases or decreases any of the properties of a UBP8rp polypeptide. As used herein, a “UBP8rp modulator” refers to a compound that increases or decreases the activity of a UBP8rp polypeptide and/or to a compound that increases or decreases the transcription level of the UBP8rp mRNA. The term “modulator” encompasses both agonists and antagonists.

As used herein, a “UBP8rp antagonist” refers to a compound that decreases the activity of a UBP8rp polypeptide and/or to a compound that decreases the expression level of the UBP8rp mRNA encoding said polypeptide. The terms “antagonist” and “inhibitor” are considered to be synonymous and can be used interchangeably throughout the disclosure.

As used herein, a “UBP8rp agonist” refers to a compound that increases the activity of a UBP8rp polypeptide and/or to a compound that Increases the expression level of the UBP8rp mRNA encoding said polypeptide. The terms “agonist” and “activator” are considered to be synonymous and can be used interchangeably throughout the disclosure.

Methods that can be used for testing modulators for their ability to increase or decrease the activity of a UBP8RP polypeptide or to increase or decrease the expression of a UBP8RP mRNA are well known in the art and further detailed below. These assays can be performed either in vitro or in vivo.

Candidate compounds according to the present invention Include naturally occurring and synthetic compounds. Such compounds include, e.g., natural ligands, small molecules, antisense mRNAs, antibodies, aptamers and small interfering RNAs. As used herein, the term “natural ligand” refers to any signaling molecule that binds to a phosphatase comprising PP2A/Bγ in vivo and includes molecules such as, e.g., lipids, nucleotides, polynucleotides, amino acids, peptides, polypeptides, proteins, carbohydrates and inorganic molecules. As used herein, the term “small molecule” refers to an organic compound. As used herein, the term “antibody” refers to a protein produced by cells of the immune system or to a fragment thereof that binds to an antigen. As used herein, the term “antisense mRNA” refers an RNA molecule complementary to the strand normally processed into mRNA and translated, or complementary to a region thereof. As used herein, the term “aptamer” refers to an artificial nucleic acid ligand (see, e.g., Ellington and Szostak (1990) Nature 346:818-822). As used herein, the term “small interfering RNA” refers to a double-stranded RNA inducing sequence-specific posttranscriptional gene silencing (see, e.g., Elbashir et al. (2001) Nature. 411:494-498).

Such candidate compounds can be obtained using any of the numerous approaches in combinatorial library methods known in the art, including, e.g., biological libraries, spatially addressable parallel solid phase or solution phase libraries, and synthetic library methods using affinity chromatography selection. The biological library approach is generally used with peptide libraries, while the other four approaches are applicable to peptide, non-peptide oligomers, aptamers or small molecule libraries of compounds.

One example of a method that may be used for screening candidate compounds for a modulator is a method comprising the steps of:
a) contacting a UBP8rp polypeptide with the candidate compound; and
b) testing the activity of said UBP8rp polypeptide in the presence of said candidate compound,
   wherein a difference in the activity of said UBP8rp polypeptide in the presence of said compound in comparison to the activity in the absence of said compound indicates that the compound is a modulator of said UBP8rp polypeptide.

Alternatively, the assay may be a cell-based assay comprising the steps of:
a) contacting a cell expressing a UBP8rp polypeptide with the candidate compound; and
b) testing the activity of said UBP8rp polypeptide in the presence of said candidate compound,
   wherein a difference in the activity of said UBP8rp polypeptide in the presence of said compound in comparison to the activity in the absence of said compound indicates that the compound is a modulator of said UBP8rp polypeptide.

The modulator may be an inhibitor or an activator. An inhibitor may decrease UBP8rp activity by, e.g., 10%, 20%, 30%, 40%, 50%, 60%, 70%, 80%, 90%, 95% or 100% compared to UBP8rp activity in the absence of said inhibitor. An activator may increase UBP8rp activity by, e.g., 10%, 20%, 30%, 40%, 60%, 60%, 70%, 80%, 90%, 95% or 100% compared to UBP8rp activity in the absence of said activator.

The modulator may modulate any activity of said UBP8RP polypeptide. The modulator may for example modulate UBP8rp mRNA expression within a cell, modulate the enzymatic activity of the UBP8rp polypeptide, or modulate binding of the UBP8rp polypeptide to its natural binding partners. Preferably, the activity of the UBP8RP polypeptide is assessed by measuring the ubiquitination state of proteins.

In one embodiment, the activity of a UBP8rp polypeptide is assessed by measuring the ubiquitin-conjugation and/or de-ubiquitination of proteins. Assays for measuring the ubiquitin-conjugation and/or de-ubiquitination of proteins are known by those of skill the art. Such assays are described, e.g., by Naviglio et al. (1998, EMBO J. 17:3241-3250) and by Gnesutta et al. (2001, J Biol. Chem. 276:39448-39454).

In a preferred embodiment, the activity of a UBP8rp polypeptide is assessed by measuring the de-ubiquitinating activity of said polypeptide. The de-ubiquitinating activity of a UBP8rp polypeptide may be measured by replacing UBP8 by a UBP8rp polypeptide in the de-ubiquitination assay described at page 3248 of Naviglio et al. (1998).

In another embodiment, the activity of a UBP8rp polypeptide is assessed by measuring the de-ubiquitinating activity of UBP8 in the presence of said UBP8rp polypeptide.

In a further preferred embodiment, the activity of a UBP8RP polypeptide is assessed by measuring the UBP8rp mRNA levels within a cell. In this embodiment, the activity can for example be measured using Northern blots, RT-PCR, quantitative RT-PCR with primers and probes specific for UBP8RP mRNAs. Alternatively, the expression of the UBP8RP mRNA is measured at the polypeptide level, by using labeled antibodies that specifically bind to the UBP8rp polypeptide in immunoassays such as ELISA assays, or RIA assays, Western blots or Immunohistochemical assays.

Modulators of UBP8rp polypeptides, which may be found, e.g., by any of the above screenings, are candidate drugs for the treatment of a chronic inflammatory disease. Thus a preferred embodiment of the present invention is the use of a UBP8rp polypeptide as a target for screening candidate compounds for candidate drugs for the treatment of a chronic inflammatory disease.

As used herein, the term “chronic inflammatory disease” refers to a chronic pathologic inflammation of a tissue or an organ of an individual. Chronic inflammatory diseases include, e.g., psoriasis, psoriatic arthritis, rheumatoid arthritis, asthma, inflammatory bowel disease and multiple sclerosis. Preferably, said chronic inflammatory disease is psoriasis.

A further aspect of the present invention is the use of a modulator of a UBP8rp polypeptide for screening for drugs for the treatment of a chronic inflammatory disease. One example of a method that can be used for screening for drugs for the treatment of a chronic inflammatory disease and/or for assessing the efficiency of an modulator of a UBP8rp polypeptide for the treatment of a chronic inflammatory disease is a method comprising the step of administering said modulator to an animal model for said chronic inflammatory disease, wherein a determination that said modulator ameliorates a representative characteristic of said chronic inflammatory disease in said animal model indicates that said modulator is a drug for the treatment of said chronic inflammatory disease. Preferably, said chronic inflammatory disease is psoriasis.

Animal models for chronic inflammatory diseases and assays for determining whether a compound ameliorates a representative characteristic of the chronic inflammatory disease In said animal model are known by those of skill in the art. A preferred animal model for psoriasis is the SCID-hu mouse that is described in Zollner et al. (2002, J Clin Invest. 109:671-679).

Determining whether the modulator ameliorates a representative characteristic of a chronic inflammatory disease may be performed using several methods available in the art. Specifically, when studying psoriasis, the representative characteristic may be the National Psoriasis Foundation Psoriasis Score (NPF-PS), the Psoriasis Area Severity Index score (PASI), or Physidan's Global Assessment score (PGA) (see, e.g., Gottlieb et al. (2003) J Drugs Dermatol. 2:260-266).

In one preferred embodiment of the present invention, the representative characteristic is the Psoriasis Area and Severity Index score. The Psoriasis Area and Severity Index is a measure of overall psoriasis severity and coverage (Fredriksson et al. (1978) Dermatologica. 157:238-244). It is a commonly used measure in clinical trials for psoriasis treatments.

In a further embodiment, a determination that a modulator of a UBP8rp polypeptide ameliorates the PASI score of an animal model for psoriasis indicates that said modulator is a drug for the treatment of psoriasis. Preferably, a 50%, 60%, 70%, 75%, 80%, 85%, 90%, 95% or greater improvement in PASI scores indicates that said modulator is a drug for the treatment of psoriasis. Most preferably, a 75% or greater improvement In PASI scores (PASI 75) indicates that said modulator is a drug for the treatment of psoriasis.

A further aspect of the present invention is directed to the use of a modulator of a UBP8rp polypeptide for preparing a medicament for the treatment of a chronic inflammatory disease. Such a medicament comprises said modulator of a UBP8rp polypeptide in combination with any physiologically acceptable carrier. Physiologically acceptable carriers can be prepared by any method known by those skilled in the art. Physiologically acceptable carriers include but are not limited to those described in Remington's Pharmaceutical Sciences (Mack Publishing Company, Easton, USA 1985). Pharmaceutical compositions comprising a modulator of a UBP8RP polypeptide and a physiologically acceptable carrier can be for, e.g., intravenous, topical, rectal, local, inhalant, subcutaneous, intradermal, intramuscular, oral, intracerebral and intrathecal use. The compositions can be in liquid (e.g., solutions, suspensions), solid (e.g., pills, tablets, suppositories) or semisolid (e.g., creams, gels) form. Dosages to be administered depend on individual needs, on the desired effect and the chosen route of administration.

As discussed in Example 7, specific variant of UBP8rp may confer abnormal proliferation capacities to psoriatic keratinocytes due to its presence in specific fraction of immuno competent cells in psoriatic lesions. Accordingly, UBP8rp antagonists are preferred candidate drugs for the treatment of a chronic inflammatory disease.

Such a medicament comprising (i) a UBP8rp modulator; or (ii) a gene therapy vector of the invention may be used in combination with any known drug for the treatment of a chronic Inflammatory disease. For example, when treating psoriasis, the modulator may be administered in combination with Raptiva, Tazarotene, Anapsos, Alefacept, Micanol, Efalith, Olopatadine, Calcipotriol, Cyclosporin A, Halobetasol propionate, Halometasone, Acitretin, GMDP, Silkis, Betamethasone mousse, Clobetasol propionate foam, Tacalcitol and/or Falecalcitriol.

The present invention further relates to the use of a UBP8rp polypeptide for screening for natural binding partners. Using a UBP8rp polypeptide as a target has a great utility for the identification of proteins involved in psoriasis and for providing new intervention points in the treatment of chronic inflammatory diseases. Such methods for screening for natural binding partners of a UBP8rp polypeptide are well known in the art. One method for the screening of a candidate substance interacting with a UBP8rp polypeptide of the present invention comprises the following steps:
a) providing a polypeptide consisting of a UBP8rp polypeptide;
b) obtaining a candidate polypeptide;
c) bringing into contact said polypeptide with said candidate polypeptide;
d) detecting the complexes formed between said polypeptide and said candidate polypeptide.

In one embodiment of the screening method defined above, the complexes formed between the polypeptide and the candidate substance are further incubated in the presence of a polyclonal or a monoclonal antibody that specifically binds to the UBP8rp polypeptide.

In a particular embodiment of the screening method, the candidate is the expression product of a DNA insert contained in a phage vector (Parmley and Smith (1988) Gene. 73:305-318). Specifically, random peptide phage libraries are used. The random DNA inserts encode for polypeptides of 8 to 20 amino acids In length (see, e.g., Oldenburg et al. (1992) Proc Natl Acad Sci USA. 89:5393-5397; Valadon et al. (1996) J Immunol Methods. 197:171-179). According to this particular embodiment, the recombinant phages expressing a polypeptide that binds to the immobilized UBP8rp polypeptide is retained and the complex formed between the UBP8rp polypeptide and the recombinant phage may be subsequently immunoprecipitated by a polyclonal or a monoclonal antibody directed against the UBP8rp polypeptide.

In a further particular embodiment of the screening method, the binding partners are identified through a two-hybrid screening assay. The yeast two-hybrid system is designed to study protein-protein interactions in vivo (Fields and Song (1989) Nature. 340:245-6), and relies upon the fusion of a bait protein to the DNA binding domain of the yeast Gal4 protein. This technique is also described in U.S. Pat. Nos. 5,667,973 and 5,283,173. The general procedure of library screening by the two-hybrid assay may for example be performed as described by Fromont-Racine et al. (1997, Nat Genet 16:277-282), the bait polypeptide consisting of a UBP8rp polypeptide. More precisely, a UBP8rp polynucleotide is fused in frame to a polynucleotide encoding the DNA binding domain of the GAL4 protein, the fused nucleotide sequence being inserted in a suitable expression vector, for example pAS2 or pM3.

In a further particular embodiment of the screening method, the binding partners are identified through affinity chromatography. The UBP8rp polypeptide may be attached to the column using conventional techniques including chemical coupling to a suitable column matrix (e.g. agarose, Affi Gel®, etc.). In some embodiments of this method, the affinity column contains chimeric proteins in which the UBP8rp polypeptide, or a fragment thereof, is fused to glutathion S transferase (GST). A mixture of cellular proteins or pool of expressed proteins as described above is applied to the affinity column. Polypeptides interacting with the UBP8rp polypeptide attached to the column can then be isolated and analyzed, e.g., on 2-D electrophoresis gel as described in Ramunsen et al., (1997, Electrophoresis, 18:588-598). Alternatively, the proteins retained on the affinity column can be purified by electrophoresis-based methods and sequenced.

In a further particular embodiment of the screening method, the binding partners are identified through optical blosensor methods (see, e.g., Edwards and Leatherbarrow, 1997). This technique permits the detection of interactions between molecules in real time, without the need of labeled molecules.

7. Biallelic Markers of the Present Invention

The present invention is directed to the use of at least one UBP8rp-related biallelic marker selected from the group consisting of the biallelic markers shown below for determining whether there is a significant association between said biallelic marker and a chronic inflammatory disease:

As used herein, the term “biallelic marker” refers to a polymorphism having two alleles at a fairly high frequency in the population, preferably a single nucleotide polymorphism. Typically the frequency of the less common allele of the biallelic markers of the present invention has been validated to be greater than 1%, preferably the frequency is greater than 10%, more preferably the frequency is at least 20% (i.e. heterozygosity rate of at least 0.32), even more preferably the frequency is at least 30% (i.e. heterozygosity rate of at least 0.42). In the present specification, the term “biallelic marker” is used to refer both to the polymorphism and to the locus carrying the polymorphism. As used herein, the term “UBP8rp-related biallelic marker” refers to a biallelic marker located in an exon of the UBP8rp gene, in an intron of the UBP8rp gene, or in regulatory regions of the UBP8rp gene. The term “UBP8rp-related biallelic marker of the present invention” refers to Biallelic markers 1, 2, 4, 6, 7, 10, 12-19, 21-30, 31-35 and 37-96 shown above and further described in Example 3.

Determining whether there is a significant association between said biallelic marker and a chronic inflammatory disease can be performed using any method well known by those of skill in the art. For example, the UBP8rp-related biallelic marker of the present invention may be genotyped in case and control populations for the inflammatory disease to be studied. The allelic frequency of markers between cases and controls may be investigated using, e.g., the Pearson Chi squared test. The EM (Expectation-Maximization) algorithm (Excoffier L & Slatkin M, 1995) may be used to estimate haplotypes for the population under investigation. Alternatively, haplotype frequency estimations may be performed by applying the OMNIBUS likelihood ratio test (PCT publication WO 01/091026). The association between UBP8rp-related biallelic markers of the present invention and psoriasis may also be performed as described by Veal et al (2002).

In all aspects and embodiments relating to UBP8rp-related biallelic markers of the present invention, the chronic inflammatory disease is preferably selected from the group consisting of psoriasis, psoriatic arthritis, rheumatoid arthritis, asthma, inflammatory bowel disease and multiple sclerosis. Most preferably, the chronic inflammatory disease is psoriasis.

The present invention is further directed to the use of at least one UBP8rp-related biallelic marker of the present invention for diagnosing whether an individual suffers from or is at risk of suffering from a chronic inflammatory disease. Specifically, the presence of allele A9 in said individual indicates that said Individual suffers from or is at risk of suffering from said chronic inflammatory disease.

In one embodiment, a single biallelic marker is used for diagnosing whether an individual suffers from or is at risk of suffering from a chronic inflammatory disease by determining the genotype of an individual. In another embodiment, a combination of several biallelic markers may be used for diagnosing whether an individual suffers from or is at risk of suffering from a chronic inflammatory disease by determining the haplotype of an individual. For example, a two-markers haplotype, a three-markers haplotype or a four-markers haplotype may be determined.

As used herein, the term “genotype” refers to the identity of the alleles present in an Individual or a sample. The term “genotype” preferably refers to the description of both copies of a single biallelic marker that are present in the genome of an individual. The individual is homozygous if the two alleles of the biallelic marker present in the genome are identical. The individual is heterozygous if the two alleles of the biallelic marker present in the genome are different.

The term “genotyping” a sample or an individual for a biallelic marker involves determining the specific alleles or the specific nucleotides carried by an individual at a biallelic marker.

As used herein, the term “haplotype” refers to a set of alleles of closely linked biallelic markers present on one chromosome and which tend to be inherited together.

Methods for determining the alleles, genotypes or haplotypes carried by an individual are well known by those of skill in the art and further detailed below.

In the context of the present invention, the individual is generally understood to be human.

UBP8rp-related biallelic markers 20 and 36 are highly associated with psoriasis, yielding p-values inferior to 10⁻⁹ (Veal et al., 2002). Thus a preferred embodiment of the present invention is directed to the use of (i) at least one UBP8rp-related biallelic marker of the present invention; and (ii) the biallelic marker 20 and/or the biallelic marker 36 for diagnosing whether an individual suffers from or is at risk of suffering from psoriasis.

As disclosed in Example 4, eleven alleles of the UBP8rp gene have been Identified in the frame of the present invention. As used herein, an “allele of UBP8rp” refers to a given variant of the UBP8rp gene. Tables 4 to 7 indicate the nucleotides that are present at UBP8 rp-related biallelic markers located between nucleotide positions 829 and 2525 of SEQ ID NO: 1. It is further demonstrated in Example 5 that allele A9, which is depicted in detail in tables 4 to 7, is found more often in Individuals suffering from psoriasis than in normal individuals.

A preferred set of UBP8rp-related biallelic markers for use in the uses and methods according to the present invention is the set of biallelic markers shown in tables 4 to 7.

The present invention is further directed to a method of genotyping comprising the steps of:
a) isolating a nucleic acid from a biological sample; and
b) detecting the nucleotide present at one or more of the UBP8rp-related biallelic markers of the present invention.

Preferably, said biological sample is derived from a single individual. It is preferred that the identity of the nucleotides at said biallelic marker is determined for both copies of said biallelic marker present in said individual's genome. In a preferred embodiment, the identity of the nucleotide at said biallelic marker is determined by a microsequencing assay. Preferably, a portion of a sequence comprising the biallelic marker is amplified prior to the determination of the identity of the nucleotide. The amplification may preferably be performed by PCR. Methods of genotyping are well known by those of skill in the art and any other known protocol may be used. The nucleotide present at a UBP8rp-related biallelic marker of the present invention may for example be determined as described in Example 3. The presence of an allele A9 in said individual indicates that said individual suffers from or is at risk of suffering from said chronic inflammatory disease.

Methods well-known to those skilled in the art that may be used for genotyping in order to detect biallelic polymorphisms include methods such as, conventional dot blot analyzes, single strand conformational polymorphism analysis (SSCP) (Orita et al. (1989) Proc Natl Acad Sci USA 86:2766-2770), denaturing gradient gel electrophoresis (DGGE) (Borresen et al. (1988) Mutat Res. 202:77-83.), heteroduplex analysis (Lessa et al. (1993) Mol Ecol. 2:119-129), mismatch cleavage detection (Grompe et al. (1989) Proc Natl Acad Sci USA. 86:5888-5892). Another method for determining the Identity of the nucleotide p resent at a particular polymorphic site employs a specialized exonuclease-resistant nucleotide derivative as described in U.S. Pat. No. 4,656,127. Oligonucleotide microarrays or solid-phase capturable dideoxynucleotides and mass spectrometry may also be used (Wen et al. (2003) World J. Gastroenterol. 9:1342-1346; Kim et al. (2003) Anal Biochem. 316:251-258). Preferred methods involve directly determining the identity of the nucleotide present at a biallelic marker site by sequencing assay, microsequencing assay, enzyme-based mismatch detection assay, or hybridization assay.

As used herein, the term “biological sample” refers to a sample comprising nucleic acids. Any source of nucleic acids, in purified or non-purified form, can be utilized as the starting nucleic add, provided it contains or is suspected of containing the specific nucleic acid sequence desired. DNA or RNA may be extracted from cells, tissues, body fluids and the like.

Methods of genotyping find use in, e.g., in genotyping case-control populations in association studies as well as in genotyping individuals in the context of detection of alleles of biallelic markers which are known to be associated with a given trait. In the context of the present invention, a preferred trait is a chronic inflammatory disease selected from the group of psoriasis, psoriatic arthritis, rheumatoid arthritis, asthma, inflammatory bowel disease and multiple sclerosis, and most preferably psoriasis.

In one embodiment, the above genotyping method further comprises the step of correlating the result of the genotyping steps with a risk of suffering from a chronic inflammatory disease.

The present invention is further directed to the use of at least one UBP8rp-related biallelic marker of the present invention for determining the haplotype of an individual. When determining the haplotype of an individual, each single chromosome should be studied independently. Methods of determining the haplotype of an individual are well known in the art and include, e.g., asymmetric PCR amplification (Newton et al. (1989) Nucleic Adds Res. 17:2503-2516; Wu et al. (1989) Proc. Natl. Acad. Sci. USE 86:2757-2760), isolation of single chromosome by limited dilution followed by PCR amplification (Ruano et al. (1990) Proc. Natl. Acad. Sci. USA. 87:6296-6300) and, for sufficiently close biallelic markers, double PCR amplification of specific alleles (Sarkar and Sommer, (1991) Biotechniques. 10:436-440).

Thus the present invention is further directed to the use of at least one UBP8rp-related biallelic marker of the present invention for determining the haplotype of an individual. For example, a method for determining a haplotype for a set of biallelic markers in an individual may comprise the steps of: a) genotyping said individual for at least one UBP8rp-related biallelic marker, b) genotyping said individual for a second biallelic marker by determining the identity of the nucleotides at said second biallelic marker. In one embodiment, both markers are UBP8rp-related biallelic markers of the present invention. In another embodiment, one marker is a UBP8rp related marker of the present invention and the other biallelic marker is biallelic marker 20 or 36.

Methods of determining a haplotype for a combination of more than two biallelic markers comprising at least one UBP8RP-related biallelic marker of the present invention in an individual are also encompassed by the present invention. In such methods, step (b) is repeated for each of the additional markers of the combination. Such a combination may comprise, e.g., 3, 4 or 5 biallelic markers.

When estimating haplotype frequencies in a population, one may use methods without assigning haplotypes to each individual. Such methods use a statistical method of haplotype determination. Thus another aspect of the present invention encompasses methods of estimating the frequency of a haplotype for a set of biallelic markers In a population, comprising the steps of: a) genotyping each individual In said population for at least one UBP8RP-related biallelic marker, b) genotyping each individual in said population for a second biallelic marker by determining the identity of the nucleotides at said second biallelic marker; and c) applying a haplotype determination method to the identities of the nucleotides determined in steps a) and b) to obtain an estimate of said frequency. Such a method may also be performed for a combination of more than 2 biallelic markers. Step (c) may be performed using any method known in the art to determine or to estimate the frequency of a haplotype in a population. Preferably, a method based on an expectation-maximization (EM) algorithm (Dempster et al. (1977) JRSSB, 39:1-38; Excoffier and Slatkin, (1995) Mol Biol Evol. 12:921-7) leading to maximum-likelihood estimates of haplotype frequencies under the assumption of Hardy-Weinberg proportions (random mating) is used for performing step (c).

Having now fully described this invention, it will be appreciated by those skilled in the art that the same can be performed within a wide range of equivalent parameters without departing from the spirit and scope of the invention and without undue experimentation.

While this invention has been described in connection with specific embodiments thereof, it will be understood that it is capable of further modifications. This application, is intended to cover any variations, uses or adaptations of the invention following, in general, the principles of the invention and including such departures from the present disclosure as come within known or customary practice within the art to which the invention pertains and as may be applied to the essential features hereinbefore set forth as follows in the scope of the appended claims.

All references cited herein, Including journal articles or abstracts, published or unpublished patent application, issued patents or any other references, are entirely Incorporated by reference herein, including all data, tables, figures and text presented in the cited references. Additionally, the entire contents of the references cited within the references cited herein are also entirely incorporated by reference.

Reference to known method steps, conventional methods steps, known methods or conventional methods is not any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various application such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### EXAMPLES

### Example 1

### Identification of the UBP8rp Gene

1.1 Isolation of the UBP8rp mRNA

RT-PCRs were performed on polyA+ RNAs from Stratagene (Reference Nos 778021 and 778022). cDNA was synthesized with the help of Rt-for-PCR kit (Clontech) with oligo(dT) and random primers for each RNA sample. The cDNA quantity obtained for the reactions varied between 0.6 and 1.5 ug of cDNA per reaction. A PCR reaction was performed using 5 μl out of 100 μl of the RT-PCR samples, primers of SEQ ID Nos. 5 and 6, and the rTth enzyme. The cycling was as follows: 94° C. 5 min; 94° C. 20sec, 67° C. 3 min-32 cycles; 72° C. 10 min.

The first PCR reaction was diluted five fold and 2% thereof was used for performing a nested PCR reaction with primers of SEQ ID Nos. 7 and 8. The cycling conditions were identical as above.

The resulting product was sequenced using primers of SEQ ID Nos. 9-35. The sequencing was carried out on ABI 377 sequencers. The sequences of the amplification products were determined using automated dideoxy terminator sequencing reactions with a dye terminator cycle sequencing protocol. The products of the sequencing reactions were run on sequencing gels and the sequences were determined using gel image analysis (ABI Prism DNA Sequencing Analysis software (2.1.2 version)).

The cDNA comprised the Open Reading Frame of SEQ ID NO: 2. This Open Reading Frame codes for a 482 amino acid long protein, the UBP8rp protein (SEQ ID NO: 3).

1.2 Identification and Annotation of the UBP8rp Gene

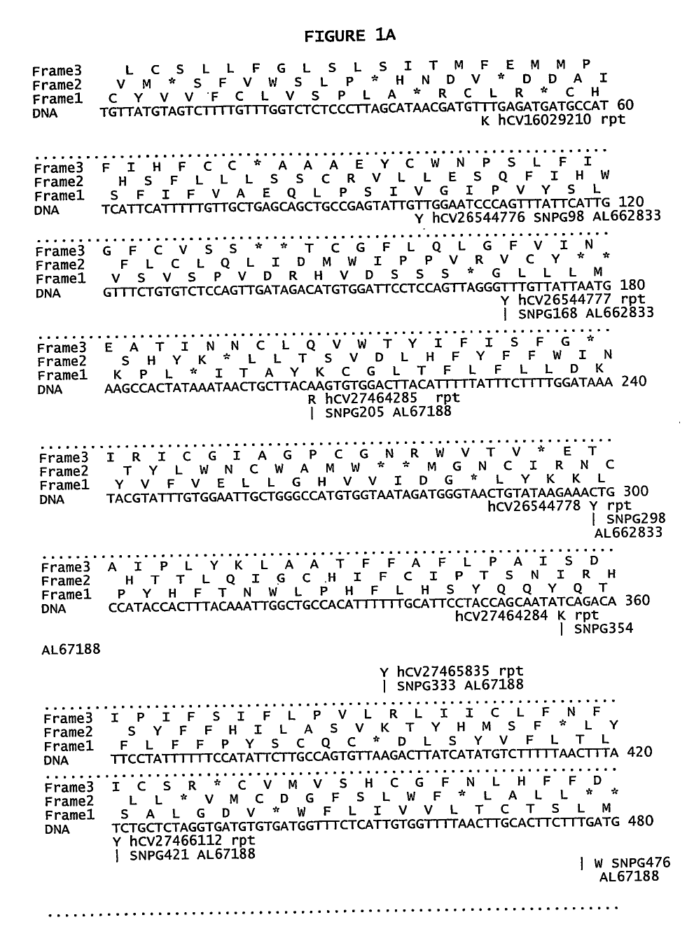

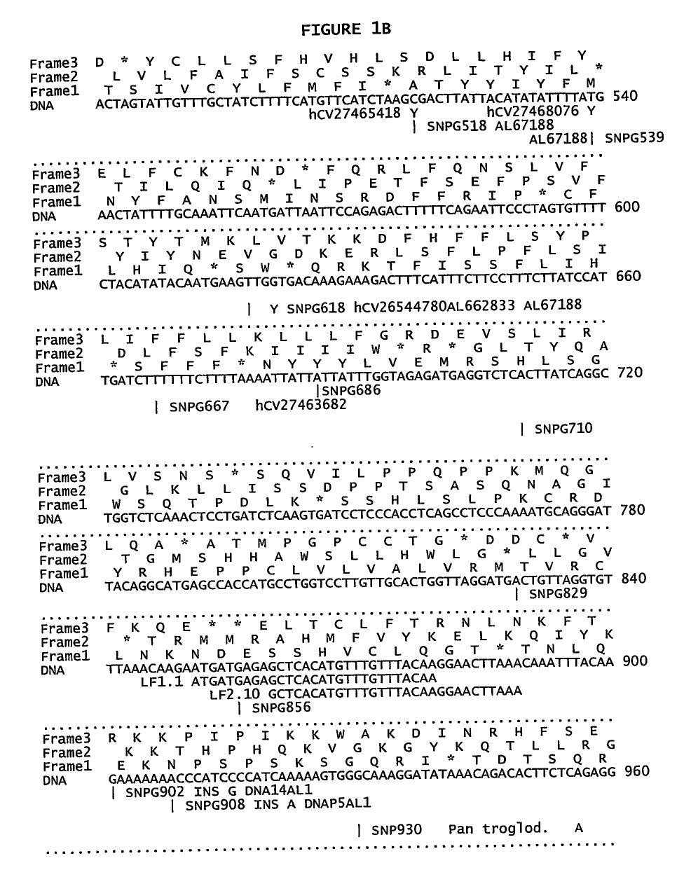

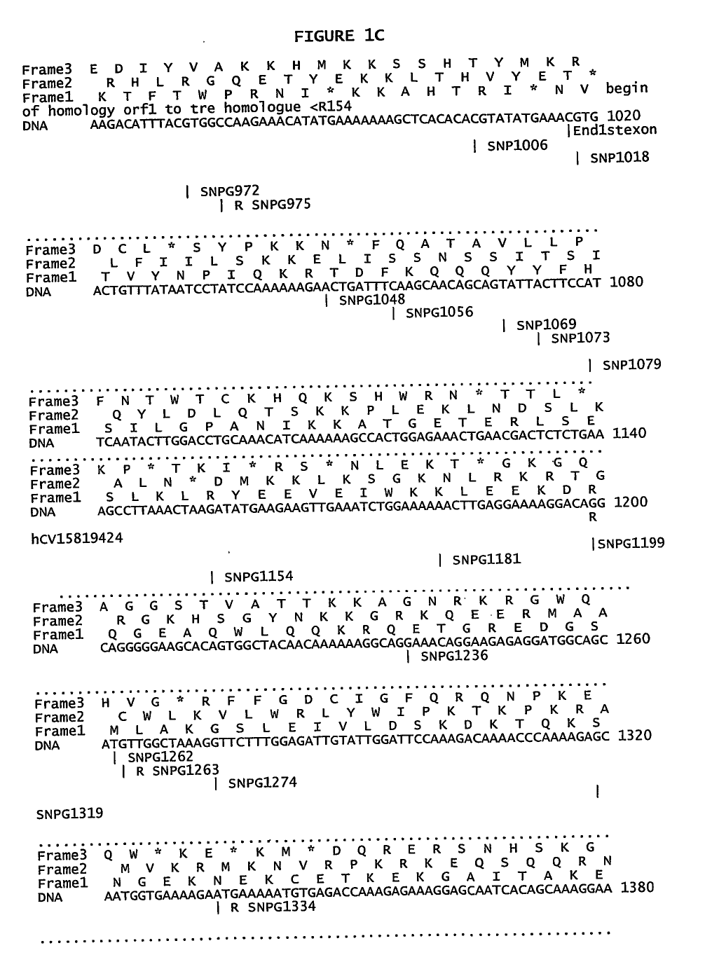

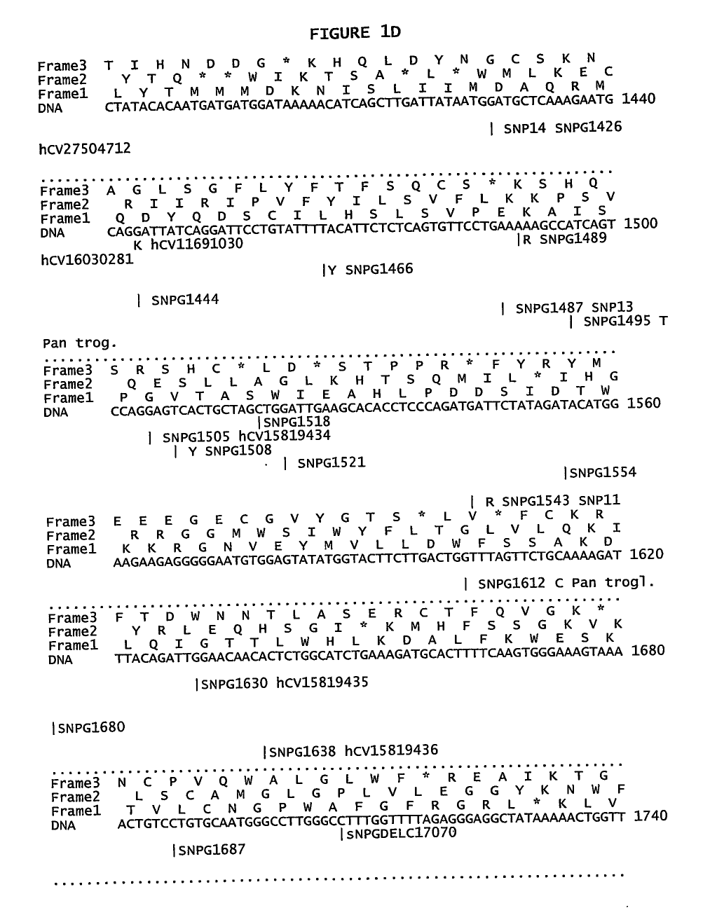

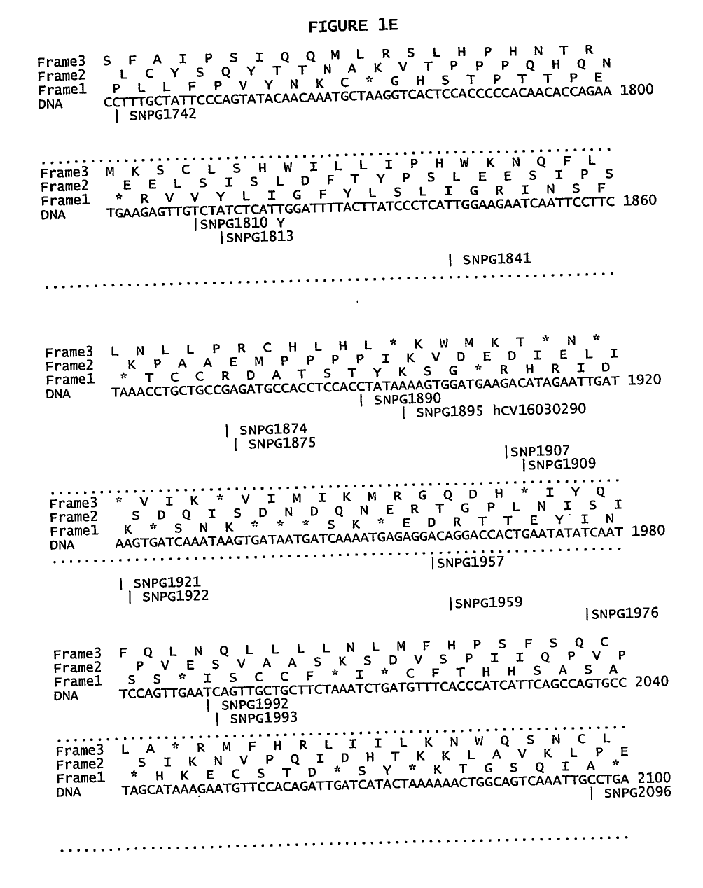

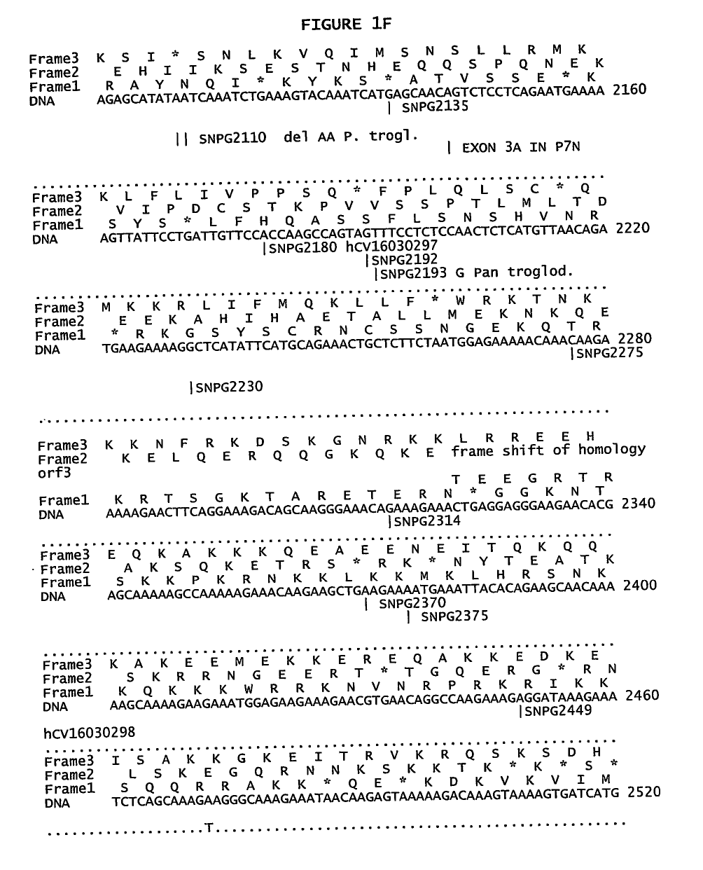

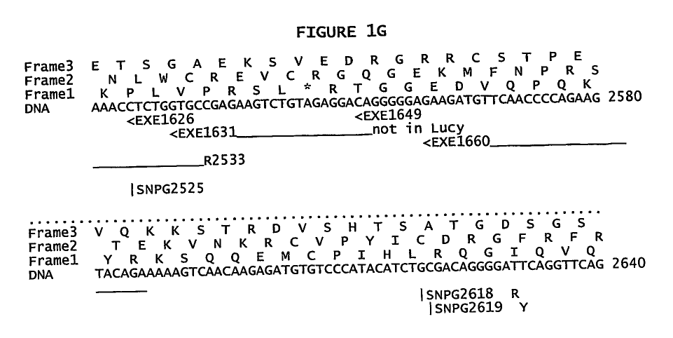

The genomic region encoding the UBP8rp protein was Identified using bioinformatic tools. The UBP8rp gene is shown as SEQ ID NO: 1. This gene is located within the 10-kb major region for susceptibility for psoriasis that was identified by Veal et al. (2002). The UBP8rp gene comprises two introns located at nucleotide positions 1018 to 1046 of SEQ ID NO: 1 and 1676 to 1718 of SEQ ID NO: 1 (see FIG. 1).

Thus it has unexpectedly been found that a novel expressed gene is located within the 10-kb major region for susceptibility for psoriasis. In prior art literature, the gene encoding UBP8rp was annotated as a silent pseudogene element not comprising any Open Reading Frame.

1.3. Analysis of the UBP8rp Protein

The UBP8rp protein shows significant homology to the UBP8 ubiquitin isopeptidase.

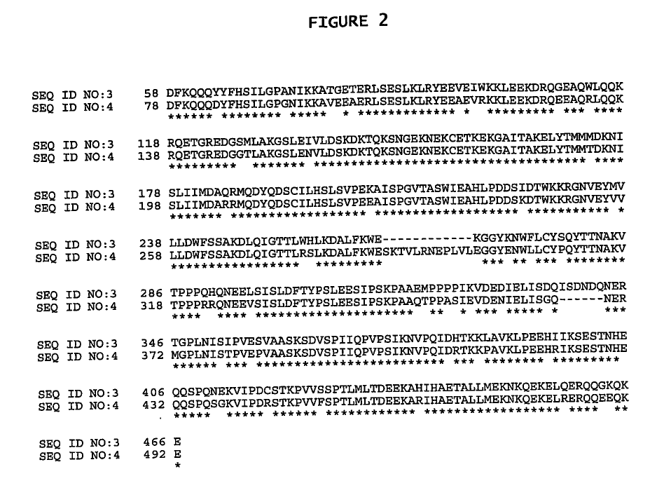

When comparing UBP8rp to UBP8 using the BLAST version 2.0 program (Altschul et al. 1990 J Mol Biol. 215:403-410), UBP8rp is found to be 74% identical to UBP8 (FIG. 2). More specifically, amino acids 57 to 466 of UBP8rp show homology to amino acids 78 to 492 of UBP8 (81% of Identity).

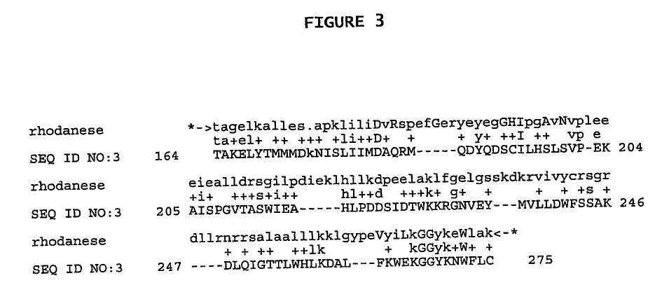

Using the SignalP and Toppred programs (Nielsen et al. 1999 Protein Engineering 12:3-9, von Heijne. 1992 J. Mol. Biol. 225:487-494), UBP8rp was found to be an intracellular protein. UBP8rp was further analyzed using the HMMER 2.1.1 program (Eddy. 1996 Current Opinion in Structural Biology 6:361-365). As shown on FIG. 3, UBP8rp displays a rhodanese-like Pfam domain at amino acid positions 164 to 433 (score: 32.7; e-value: 8.3e-06). The presence of rhodanese-like domains is a common feature to UBPs since UBP8 and the ubiquitin isopeptidase 7 from Saccharomyces cerevisiae also display a single rhodanese-like domain.

Thus UBP8rp is a novel member of the UBP family. UBP8rp seems to belong to the ubiquitin-proteasome pathway, and may play a role in the selective degradation of intracellular proteins.

### Example 2

### Analysis of UBP8rp Expression by Quantitative PCR

2.1. Expression of UBP8rp in Different Tissues

The expression levels of UBP8rp in adult skin, fetal skin, testis, brain, adipose tissue, small intestine and colon was determined using commercial total RNA (Clontech). In addition, the expression levels of UBP8rp in adult skin was also determined using skin biopsies from I'Hôpital Pasteur (Paris, France).

20 μL of commercial total RNA were treated by 4 units of RNase free DNAse I (Ambion). The cDNA was obtained using the “Advantage RT for PCR” kit (Clontech) following the Instructions provided by the supplier. The Quantitative PCR was performed using the TaqMan Universal PCR Master mix NO AmpErase UNG (Applied Biosystems). The reaction was performed with 25 ng of cDNA, 300 nM of each primer and 200 nM of Taqman probe. The program applied was: 40 to 50 cycles at 95° C. for 10 minutes; 95° C. for secondes; 60° C. for 1 minute.

The experiments were performed on a 7900HT Applied Biosystems machine. Each experiment was performed either with primers of SEQ ID Nos. 36-38 or with primers of SEQ ID Nos. 39-41, as detailed In table 1 below. The efficiency of the chosen primers were calculated as described in the User Bulletin Applied Biosystems (1997—updated 10/2001) ABI PRISM 7700 Sequence Detection System. Relative Quantification of Gene Expression. User Bulletin #2.

TABLE 1A

| | Primer | Primer | Taqman | Amplicon |
|---|---|---|---|---|
| | forward | reverse | MGB probe | length |
| 1^{st} set of primers | SEQ ID NO: 36 | SEQ ID NO: 37 | SEQ ID NO: 38 | 98 |
| 2^{nd} set of primers | SEQ ID NO: 39 | SEQ ID NO: 40 | SEQ ID NO: 41 | 109 |

The expression was calculated as described by Livak & Schmittgen (2001, Methods 25:402-408) The Ct is an absolute value indicating the relative expression level of a gene. A Ct under 20 is indicative of a highly expressed gene. A Ct between 35 and 40 is indicative of a weakly expressed gene. Calculation of the 2^{-ΔΔCt} value allows to compare expression levels of a gene in a target tissue to be studied and in a reference tissue.

In order to confirm that the primers specifically amplify UBP8rp and not another gene, the amplicons obtained by quantitative PCR were sequenced with the forward and reverse primers used for performing the QPCR. It was found that the cDNA amplified by PCR effectively corresponds to the UBP8rp cDNA.

The results of the quantitative expression analysis are shown in table 2. The 2^{-ΔΔCt} value was calculated using testis as a reference tissue, numerous genes being expressed in testis at high levels.

TABLE 1B

| | Primers | | | | | |
|---|---|---|---|---|---|---|
| | SEQ ID Nos. 36-38 | | | SEQ ID Nos. 39-41 | | |
| | effi- | | | effi- | | |
| Tissue | ciency | Ct | 2^{-ΔΔCt} | ciency | Ct | 2^{-ΔΔCt} |
| Testis | — | 36.1 | 1.0 | — | 35.4 | 1.0 |
| Adult skin (biopsy) | — | 36.1 | 2.5 | — | 36.6 | 1.1 |
| Adult skin | — | 39.6 | −5.3 | — | 39.5 | −7.8 |
| (commercial) | | | | | | |
| Fetal skin | 104% | 36.7 | −1.0 | 77% | 37.9 | −3.6 |
| Brain | 259% | 38.9 | −8.3 | 342% | 38.7 | −11.5 |
| Adipose | — | 38.3 | −4.7 | — | 38.3 | −7.0 |
| Small intestin | — | 38.3 | −2.4 | — | 39.0 | −6.1 |
| Colon | — | 37.2 | −1.7 | — | 36.6 | −1.8 |

Using commercially available RNA, UBP8rp is found to be significantly expressed in testis, foetal skin, and colon, although at a low level. In addition, UBP8rp is found to be expressed at a very low level in adult skin, brain, adipose and small intestin.

When using RNA from skin biopsies, UBP8rp is found to be significantly expressed in adult skin. Specifically, expression of UBP8rp is found to be higher in adult skin than In any other tissue, both with primers of SEQ ID Nos. 36-38 and with primers of SEQ ID Nos. 39-41. When the experiment is performed using primers SEQ ID Nos. 36-38, expression of UBP8rp is found to be 2.5-fold higher in adult skin than in testis.

2.2. Expression of UBP8rp During Development of Keratinocytes

The expression of UBP8rp In keratinocytes under development was studied. Keratinocyte cells supplied by the company Skinethic (Nice, France) in various time of cellular development. day 3 (D3), day 5 (D5), day 7 (D7), day 10 (D10), day 13 (D13) and day 17 (D17). The expression levels of UBP8rp, UBP8, cytokeratin 15 and Psoriasin were determined by quantitative PCR.

Total RNA from the different keratinocytes cultures was extracted according to the instructions provided by the RNeasy® Mini kit Qiagen. DNA contamination was removed from the RNA by DNase I (Qiagen) and the sample was resuspended in DEPC (diethyl pyrocarbonate) treated water. Concentration was determined by spectrophotometry 260/280 nm using a GeneQuant pro RNA-DNA calculator (Amersham Pharmacia Biotech). The quality were determined using RNA 6000 Nano Assay Labchipt and Reagents (Agilent Technologies, Waldbronn, Germany) on the Agilent 2100 bioanalyzer according to the supplier's instructions.

The cDNA was obtained using “Advantage RT for PCR” kit (Clontech) following the instructions provided by the supplier. The quantitative PCR was performed using the TaqMan Universal PCR Master mix NO AmpErase UNG (Applied Biosystems) for UBP8 and UBP8rp genes and SYBR Green PCR Master Mix (Applied Biosystems) for Cytokeratine 15 and Psoriasin. The program applied was: 40 cycles at 95° C. for 10 minutes; 95° C. for 15 secondes; 60° C. for 1 minute.

The experiments were performed on a 7900HT Applied Biosystems machine. Each experiment was performed either with primers of SEQ ID as detailed in table 2 below. The efficiency of the chosen primers were calculated as described in the User Bulletin Applied Biosystems (1997—updated 1012001) ABI PRISM 7700 Sequence Detection System. Relative Quantification of Gene Expression. User Bulletin #2.

TABLE 2A

| | | Amplicon |
|---|---|---|
| Genes | Primers | length |
| UBP8 | SEQ ID Nos. 58, 59 and 60 | 107 |
| UBP8rp | SEQ ID NO: 36, 37 and 38 | 98 |
| Psoriasin | SEQ ID Nos. 61, 62 and 63 | 73 |
| Cytokeratin 15 | SEQ ID Nos. 64, 65 and 66 | 72 |

The expression was calculated as described by Livak & Schmittgen (2001, Methods 25:402-408) The Ct is an absolute value Indicating the relative expression level of a gene. Calculation of the 2^{-ΔΔCt} value allows to compare expression levels of a gene in a target tissue to be studied and in a reference tissue.

The genes Cytokeratin 15 and Psoriasin (also named S100A7) were used as controls. Psoriasin was used since it is known to be up-regulated in psoriasis (Semprini et al. (2002) Hum Genet. 111:310-313). Cytokeratin 15 was used since it is a marker of keratinocyte differentiation. Cytokeratin 16 and cytokeratin 17 were also tested as controls, but cytokeratin 15 gave the most reproducible results. (Leube et al. (1988) J Cell Biol. 106:1249-1261).

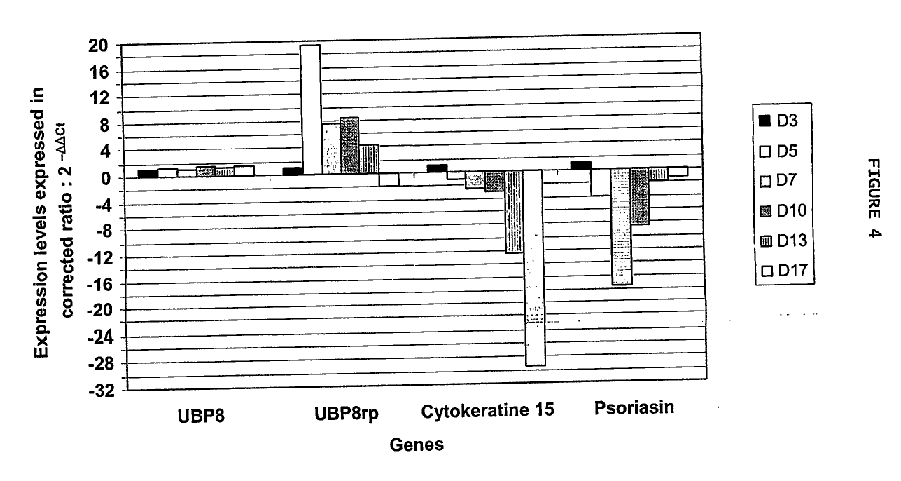

The results are shown in table 2B and on FIG. 4.

TABLE 2B

| | UBP8 | UBP8rp | Cytokeratine 15 | Psoriasin |
|---|---|---|---|---|
| Raw Ratio: | | | | |
| 2^{-ΔΔCt} | | | | |
| D3 | 1.00 | 1.00 | 1.00 | 1.00 |
| D5 | 1.36 | 19.30 | 0.87 | 0.24 |
| D7 | 1.09 | 7.57 | 0.38 | 0.06 |
| D10 | 1.45 | 8.30 | 0.33 | 0.12 |
| D13 | 1.37 | 4.22 | 0.08 | 0.52 |
| D17 | 1.48 | 0.51 | 0.03 | 0.79 |
| Ratio corrected: | | | | |
| 2^{-ΔΔCt} | | | | |
| D3 | 1.00 | 1.00 | 1.00 | 1.00 |
| D5 | 1.36 | 19.30 | −1.15 | −4.12 |
| D7 | 1.09 | 7.57 | −2.64 | −17.67 |
| D10 | 1.45 | 8.30 | −3.05 | −8.58 |
| D13 | 1.37 | 4.22 | −12.43 | −1.91 |
| D17 | 1.48 | −1.98 | −29.40 | −1.26 |

The raw ratio data given in table 2B show for example that:
UBP8 expression is up-regulated 1.36 fold at day 5 compared to its expression at day 3; and
Cytokeratine 15 expression is up-regulated 0.87 fold at day 5 compared to its expression at day 3.

It can be concluded from FIG. 4 that the transcription level of UBP8rp is modulated during growth and development of normal human keratinocytes in culture. Psoriasis being an inflammatory skin disorder characterized by keratinocyte hyper-proliferation and altered differentiation of keratinocytes, these results further indicate that UBP8rp may play a role in psoriasis.

### Example 3

### Identification of Biallelic Markers Located in the UBP8rp Gene

BM Nos. 17-19, 22, 25, 27-29, 31-35 and 37-101 were identified as detailed below. Fifteen biallelic markers were identified using sequence data provided by Celera (BM Nos. 1, 2, 4, 6, 7, 10, 12-16, 21, 23, 24 and 26).

50 to 100 ng of genomic DNA from lymphoblastoid cell lines Lucy or Boleth (CEPH collection) were used to perform a PCR reaction with primers of SEQ ID Nos. 42 and 43. The PCR assays were performed using the following protocol:
5 units of Amplitaq enzyme (Perkin-Elmer, No. 808-0101)
30 μl of reaction mix with 10× supplied Taq buffer
250 μM each dNTP,
15 μM of each primer
cycling: 94° C. 10 min, then 30 cycles of 3 steps—94° C. 30sec.; 55° C. 30sec; 72° C. 30sec, then 72° C. 10 min
   The PCR product was sequenced with the help of amplification primers. The sequences were blasted against genomic sequence, and sequence curves were compared. Biallelic marker Nos. 17-19 and 22 were thus identified.

50-100 ng of genomic DNA from lymphoblastoid cell lines Lucy or Boleth (CEPH collection) were used to perform a long-range PCR reaction with primers of SEQ ID Nos. 44 and 45. The PCR assays were performed using the following protocol:
2 units of rTTh XL enzyme (Perkin-Elmer)
50 μl of reaction mix with supplied 3,3× buffer and 200 μM of each dNTP
20 μM of each primer
1.1 mM Mg0Ac.
Cycling: 94° C. 5 min, then 32 cycles of 2 steps—94° C. 20sec; 66° C. 4 min, then 72° C. 10 min.

The resulting product was sequenced with the help of the following pairs of primers: SEQ ID Nos. 46 and 47, SEQ ID Nos. 48 and 49, and SEQ ID Nos. 50 and 51. The sequences were compared by blast and by manual inspection of sequence electrophoregrams.

Sequencing by SEQ ID Nos. 46 and 47 allowed the identification of biallelic marker NO: 25. Sequencing by SEQ ID Nos. 48 and 49 allowed the identification of biallelic markers Nos. 27-29 and 31. Sequencing by SEQ ID Nos. 50 and 51 allowed the identification of biallelic markers Nos. 27-29 and 31.

Genomic DNA samples from Individuals suffering from psoriasis and from control individuals were cloned and sequenced with SEQ ID Nos. 67 to 77.

These samples correspond to:
19 skin biopsies from Russian individuals suffering from posriasis. These individuals were undergoing a clinical trial for assessing the efficiency of Onercept.
20 samples of DNA from blood from Russian individuals who do not suffer from schizophrenia. It is not known whether these individuals suffer from psoriasis or not.
5 skin samples from individuals on whom a chirurgic intervention has been performed. It is not known whether these individuals suffer from psoriasis or not.

Sequencing of these samples allowed the identification of biallelic markers Nos. 42-101.

The alternative alleles of biallelic markers Nos. 1-101 and their location within the UBP8rp gene are indicated in table 3. Biallelic markers Nos. 20 and 36, which are known to be highly associated with psoriasis, are shown in bold letters.

TABLE 3

| BM | Internal | Position on | located in | | Alternative | Sequence | Sequence |
|---|---|---|---|---|---|---|---|
| No. | designation | SEQ ID NO: 1 | exon No. | Coding | nucleotides | in Boleth | in Lucy |
| 1 | hCV15819424 | 1199 | — | — | A/G | — | — |
| 2 | hCV16030280 | 1262 | 2 | Yes | C/T | — | — |
| 3 | SNP n.14 | 1426 | 2 | Yes | C/G | — | — |
| 4 | hCV11691030 | 1444 | 2 | Yes | G/T | — | — |
| 5 | SNP n.13 | 1487 | 2 | Yes | A/G | — | — |
| 6 | hCV16030281 | 1490 | 2 | Yes | A/G | — | — |
| 7 | hCV15819434 | 1505 | 2 | Yes | G/T | — | — |
| 8 | SNP n.12 | 1518 | 2 | Yes | C/T | — | — |
| 9 | SNP n.11 | 1554 | 2 | No | C/T | — | — |
| 10 | hCV15819435 | 1630 | 2 | Yes | A/G | — | — |
| 11 | SNP n.10 | 1638 | 2 | No | A/T | — | — |
| 12 | hCV16030289 | 1680 | — | — | A/G | — | — |
| 13 | hCV16030290 | 1895 | 3 | Yes | A/G | — | — |
| 14 | hCV16030297 | 2180 | 3 | Yes | A/G | — | — |
| 15 | hCV16030298 | 2449 | — | — | A/T | — | — |
| 16 | hCV16030299 | 2721 | — | — | G/T | — | — |
| 17 | SNPG3127 | 3127 | — | — | A/G | A/A | A/G |
| 18 | SNPG3137 | 3137 | — | — | C/T | T/T | C/T |
| 19 | SNPG3138 | 3138 | — | — | A/G | G/G | G/A |
| 20 | SNP n.9 | 3183 | — | — | A/G | A/A | A/G |
| 21 | hCV15824895 | 3222 | — | — | C/G | G/G | G/G |
| 22 | SNPG3269 | 3269 | — | — | C/T | C/T | T/T |
| 23 | hCV15824896 | 3445 | — | — | C/T | — | — |
| 24 | hCV16030306 | 3470 | — | — | A/G | — | — |
| 25 | SNPG3915 | 3915 | — | — | C/T | T/T | C/C |
| 26 | hCV16030307 | 3973 | — | — | A/C | A/A | A/A |
| 27 | SNPG4254 | 4254 | — | — | A/G | A/A | A/A |
| 28 | SNPG4472 | 4472 | — | — | A/T | A/A | A/A |
| 29 | SNPG4660 | 4660 | — | — | C/T | C/C | C/T |
| 30 | SNP n.8 | 4770 | — | — | A/G | G/G | A/A |
| 31 | SNPG4919 | 4919 | — | — | A/G | G/G | A/G |
| 32 | SNPG4973 | 4973 | — | — | C/T | T/T | C/T |
| 33 | SNPG5063 | 5063 | — | — | C/T | T/T | C/C |
| 34 | SNPG5065 | 5065 | — | — | G/T | G/G | T/T |
| 35 | SNPG5079 | 5079 | — | — | C/T | C/C | C/T |
| 36 | SNP n.7 | 5080 | — | — | C/T | C/C | C/T |
| 37 | SNPG5088 | 5088 | — | — | C/G | G/G | C/C |
| 38 | SNPG5090 | 5090 | — | — | C/T | T/T | C/C |
| 39 | SNPG5407 | 5407 | — | — | C/T | C/C | C/T |
| 40 | SNPG5466 | 5466 | — | — | A/G | G/G | G/G |
| 41 | SNPG5520 | 5520 | — | — | C/T | T/T | T/T |
| 42 | SNPG829 | 829 | — | | A/G | | |
| 43 | SNPG856 | 856 | 1 | | A/G | | |
| 44 | SNPG902 | 902 | 1 | | insertion of G | | |
| 45 | SNPG908 | 908 | 1 | | insertion of A | | |
| 46 | SNPG972 | 972 | 1 | | A/G | | |
| 47 | SNPG975 | 975 | 1 | | A/G | | |
| 48 | SNPG1006 | 1006 | 1 | | C/T | | |
| 49 | SNPG1018 | 1018 | — | | A/G | | |
| 50 | SNPG1048 | 1048 | 2 | | A/C | | |
| 51 | SNPG1056 | 1056 | 2 | | C/T | | |
| 52 | SNPG1069 | 1069 | 2 | | G/T | | |
| 53 | SNPG1073 | 1073 | 2 | | A/G | | |
| 54 | SNPG1079 | 1079 | 2 | | A/G | | |
| 55 | SNPG1108 | 1108 | 2 | | A/G | | |
| 56 | SNPG1154 | 1154 | 2 | | A/G | | |
| 57 | SNPG1181 | 1181 | 2 | | A/G | | |
| 58 | SNPG1236 | 1236 | 2 | | A/G | | |
| 59 | SNPG1263 | 1263 | 2 | | A/G | | |
| 60 | SNPG1274 | 1274 | 2 | | A/G | | |
| 61 | SNPG1319 | 1319 | 2 | | G/T | | |
| 62 | SNPG1334 | 1334 | 2 | | A/G | | |
| 63 | SNPG1444 | 1444 | 2 | | G/T | | |
| 64 | SNPG1466 | 1466 | 2 | | C/T | | |
| 65 | SNPG1489 | 1489 | 2 | | A/G | | |
| 66 | SNPG1508 | 1508 | 2 | | C/T | | |
| 67 | SNPG1521 | 1521 | 2 | | G/T | | |
| 68 | SNPG1543 | 1543 | 2 | | A/G | | |
| 69 | SNPG1687 | 1687 | — | | A/C | | |
| 70 | SNPG1707 | 1707 | — | | deletion of C | | |
| 71 | SNPG1728 | 1728 | 3 | | A/G | | |
| 72 | SNPG1742 | 1742 | 3 | | C/T | | |
| 73 | SNPG1810 | 1810 | 3 | | C/T | | |
| 74 | SNPG1813 | 1813 | 3 | | A/C | | |
| 75 | SNPG1841 | 1841 | 3 | | C/T | | |
| 76 | SNPG1874 | 1874 | 3 | | C/G | | |
| 77 | SNPG1875 | 1875 | 3 | | A/G | | |
| 78 | SNPG1890 | 1890 | 3 | | A/C | | |
| 79 | SNPG1907 | 1907 | 3 | | A/G | | |
| 80 | SNPG1909 | 1909 | 3 | | C/T | | |
| 81 | SNPG1921 | 1921 | 3 | | A/C | | |
| 82 | SNPG1922 | 1922 | 3 | | A/G | | |
| 83 | SNPG1957 | 1957 | 3 | | A/G | | |
| 84 | SNPG1959 | 1959 | 3 | | A/G | | |
| 85 | SNPG1976 | 1976 | 3 | | C/T | | |
| 86 | SNPG1992 | 1992 | 3 | | C/T | | |
| 87 | SNPG1993 | 1993 | 3 | | C/T | | |
| 88 | SNPG2096 | 2096 | 3 | | C/G | | |
| 89 | SNPG2135 | 2135 | 3 | | A/G | | |
| 90 | SNPG2192 | 2192 | 3 | | A/G | | |
| 91 | SNPG2230 | 2230 | 3 | | C/G | | |
| 92 | SNPG2275 | 2275 | 3 | | C/T | | |
| 93 | SNPG2314 | 2314 | 3 | | A/G | | |
| 94 | SNPG2370 | 2370 | — | | A/C | | |
| 95 | SNPG2375 | 2375 | — | | A/T | | |
| 96 | SNPG2525 | 2525 | — | | C/T | | |

### Example 4

### Identification of Eleven Different Alleles

The identity of the nucleotide at each UBP8rp-related biallelic marker for each of the above samples is indicated in tables 4 to 7.

The header column indicates the name of the sample.

Samples from Russian individuals suffering from posriasis are identified by a name beginning with “P”. Samples from Russian individuals who do not suffer from schizophrenia are identified by a name beginning with “DNA” in the tables 4-7 below. Samples from individuals on whom a chirurgic intervention has been performed are identified by a name beginning with “S” in the tables 4-7 below. The sequence found for the lymphoblastoid cell lines Lucy and Boleth is further indicated.

The letter “P”, “DNA” or “S” is followed by two numbers. The first number indicates an identification number of the sample (e.g., “S4” stands for sample No. 4 of the samples coming from individuals on whom a chirurgic intervention has been performed). Since UBP8rp is located on an autosome, each individual possesses two copies of the UBP8rp gene. Thus the second number indicates whether the sequence was found for the “first” or the “second” copy of the UBP8rp gene.

Finally, the table further lists genomic sequences available in databases (Accession No. indicated as a name).

The header row indicates the name of the biallelic marker (corresponding to the internal designation in table 3). The number corresponds to the position of the biallelic marker on SEQ ID NO: 1.

The second row indicates the polymorphic variation. The standard PCT nomenclature has been used. The “N+” means that the polymorphic variation corresponds to an insertion of a “N” nucleotide after the given position on SEQ ID NO: 1. In the corresponding column, the presence of a “0” indicates that the allele does not exhibit an insertion. The “dN” means that the polymorphic variation corresponds to a deletion of a ‘N’ nucleotide. In the corresponding column, the presence of a “0” Indicates that the allele exhibits the deletion.

The third row Indicates the amino acid change (if any). The term “ORF-” indicates that the open reading frame is changed further to the polymorphic variation. The term “SSP” indicates that the polymorphic change is located within a consensus for splicing of the pre-messenger RNA.

Nucleotides indicated in bold correspond to a nucleotide that is different to the one found at the same location on SEQ ID NO: 1.

The alignment of all these sequences allowed the identification of several alleles of the UBP8rp gene, which have been classified into eleven “major alleles”: alleles A1 to A11. Some of the “major alleles” are further classified into “sub-alleles”. “Sub-alleles” are different by only one SNP from a ‘major allele’. For example, allele A9 can be classified into three “sub-alleles”, referred to as A9.1, A9.2 and A9.3. The name of the allele is indicated in the second column of tables 4-7.

TABLE 4

| | | SNP | | | | | | |
|---|---|---|---|---|---|---|---|---|
| | | | SNPG856 | SNPG902 | SNPG908 | SNPG972 | SNPG975 | SNPG1006 |
| | | SNPG829 | R | G+ | A+ | R | R | Y |
| AA CHANGE | alllele | R | Mi/I | ORF- | ORF- | R/H | G/D | 12SS |
| Boleth.1 | A1.1 | A | G | 0 | 0 | G | G | T |
| Boleth.2 | A1.1 | A | G | 0 | 0 | G | G | T |
| P19.1 | A1.1 | A | G | 0 | 0 | G | G | T |
| DNA14.1 | A1.2 | A | G | G | 0 | G | G | T |
| P10.1 | A1.2 | A | G | G | 0 | G | G | T |
| P1.1 | A11 | A | G | 0 | 0 | G | G | C |
| LA1 | A2 | A | G | 0 | 0 | G | A | C |
| P1.2 | A3 | A | G | 0 | 0 | G | G | C |
| P5.2 | A3 | A | G | 0 | 0 | G | G | C |
| AC004204 | A3 | A | G | 0 | 0 | G | G | C |
| BX248310 | A3 | A | G | 0 | 0 | G | G | C |
| AL84544 | A3 | A | G | 0 | 0 | G | G | C |
| S2AG | A3 | A | G | 0 | 0 | G | G | C |
| DNA5.1 | A3 | A | G | 0 | 0 | G | G | C |
| DNA4.1 | A3 | A | G | 0 | 0 | G | G | C |
| DNA7.11 | A3 | A | G | 0 | 0 | G | G | C |
| DNA9.1 | A3 | A | G | 0 | 0 | G | G | C |
| DNA15.1 | A3 | A | G | 0 | 0 | G | G | C |
| DNA15.2 | A3 | A | G | 0 | 0 | G | G | C |
| DNA19.1 | A3 | A | G | 0 | 0 | G | G | C |
| AL662833 | A4.1 | A | G | 0 | 0 | G | G | C |
| AP000508 | A4.1 | A | G | 0 | 0 | G | G | C |
| S5.2 | A4.1 | A | G | 0 | 0 | G | G | C |
| S4.1 | A4.2 | A | G | 0 | 0 | G | G | C |
| S4.2 | A4.2 | A | G | 0 | 0 | G | G | C |
| DNA8 | A4.1 | A | G | 0 | 0 | G | G | C |
| DNA8.2 | A4.1 | A | G | 0 | 0 | G | G | C |
| DNA12.1 | A4.1 | A | G | 0 | 0 | G | G | C |
| P2.1 | A4.1 | A | G | 0 | 0 | G | G | C |
| P16.1 | A4.1 | A | G | 0 | 0 | G | G | C |
| P5.1 | A5.2 | A | G | 0 | A+ | G | G | C |
| P9.1 | A5.1 | A | G | 0 | A+ | G | G | C |
| P6.2 | A12 | G | G | 0 | 0 | G | G | C |
| S1AC | A6 | G | A | 0 | 0 | G | G | C |
| S2CA | A6 | G | A | 0 | 0 | G | G | C |
| S3.1 | A6 | G | A | 0 | 0 | G | G | C |
| DNA1.1 | A6 | G | A | 0 | 0 | G | G | C |
| DNA1.2 | A6 | G | A | 0 | 0 | G | G | C |
| DNA6.1 | A6 | G | A | 0 | 0 | G | G | C |
| DNA9.2 | A6 | G | A | 0 | 0 | G | G | C |
| DNA10 | A6 | G | A | 0 | 0 | G | G | C |
| DNA10.2 | A6 | G | A | 0 | 0 | G | G | C |
| DNA17.1 | A6 | G | A | 0 | 0 | G | G | C |
| DNA13.1 | A6 | G | A | 0 | 0 | G | G | C |
| P12.1 | A6 | G | A | 0 | 0 | G | G | C |
| AL67188 | A6 | G | A | 0 | 0 | G | G | C |
| Lucy.1 | A6 | G | A | 0 | 0 | G | G | C |
| DNA18.1 | A8 | G | G | 0 | 0 | G | G | C |
| DNA18.2 | A8 | G | G | 0 | 0 | G | G | C |
| DNA19.2 | A8 | G | G | 0 | 0 | G | G | C |
| DNA3 | A8 | G | G | 0 | 0 | G | G | C |
| DNA3 | A8 | G | G | 0 | 0 | G | G | C |
| P14.1 | A8 | G | G | 0 | 0 | G | G | C |
| P4.1 | A8 | G | G | 0 | 0 | G | G | C |
| P7.1 | A8 | G | G | 0 | 0 | G | G | C |
| DNA11.2 | A9.2 | G | G | 0 | 0 | G | G | C |
| DNA12.2 | A9.1 | G | G | 0 | 0 | G | G | C |
| DNA14.2 | A9.1 | G | G | 0 | 0 | G | G | C |
| DNA16.1 | A9.1 | G | G | 0 | 0 | G | G | C |
| DNA16.2 | A9.1 | G | G | 0 | 0 | G | G | C |
| DNA17.2 | A9.1 | G | G | 0 | 0 | G | G | C |
| DNA2.2 | A9.3 | G | G | 0 | 0 | G | G | C |
| DNA20.1 | A9.1 | G | G | 0 | 0 | G | G | C |
| DNA20.2 | A9.1 | G | G | 0 | 0 | G | G | C |
| DNA4.2 | A9.1 | G | G | 0 | 0 | G | G | C |
| DNA6.2 | A9.1 | G | G | 0 | 0 | G | G | C |
| LucyA22 | A9.1 | G | G | 0 | 0 | G | G | C |
| P12.2 | A9.1 | G | G | 0 | 0 | G | G | C |
| P13.1 | A9.1 | G | G | 0 | 0 | G | G | C |
| P13.2 | A9.1 | G | G | 0 | 0 | G | G | C |
| P14.2 | A9.1 | G | G | 0 | 0 | G | G | C |
| P15.2 | A9.1 | G | G | 0 | 0 | G | G | C |
| P16.2 | A9.1 | G | G | 0 | 0 | G | G | C |
| P17.2 | A9.1 | G | G | 0 | 0 | G | G | C |
| P18.1 | A9.1 | G | G | 0 | 0 | G | G | C |
| P18.2 | A9.2 | G | G | 0 | 0 | G | G | C |
| P19.2 | A9.2 | G | G | 0 | 0 | G | G | C |
| P2.2 | A9.1 | G | G | 0 | 0 | G | G | C |
| P3.1 | A9.1 | G | G | 0 | 0 | G | G | C |
| P3.2 | A9.2 | G | G | 0 | 0 | G | G | C |
| P4.2 | A9.1 | G | G | 0 | 0 | G | G | C |
| P6.1 | A9.1 | G | G | 0 | 0 | G | G | C |
| P7.2 | A9.3 | G | G | 0 | 0 | G | G | C |
| P8.1 | A9.1 | G | G | 0 | 0 | G | G | C |
| P8.2 | A9.1 | G | G | 0 | 0 | G | G | C |
| P9.2 | A9.2 | G | G | 0 | 0 | G | G | C |
| S5.1 | A9.1 | G | G | 0 | 0 | G | G | C |
| DNA11.1 | A10 | G | G | 0 | 0 | A | G | C |
| DNA13.2 | A10 | G | G | 0 | 0 | A | G | C |
| P10.2 | A10 | G | G | 0 | 0 | A | G | C |
| P15.1 | A10 | G | G | 0 | 0 | A | G | C |
| P17.1 | A10 | G | G | 0 | 0 | A | G | C |
| P11.1 | A10 | G | G | 0 | 0 | A | G | C |
| P11.2 | A10 | G | G | 0 | 0 | A | G | C |
| DNA5.2 | A10 | G | G | 0 | 0 | A | G | C |
| DNA2.1 | A10 | G | G | 0 | 0 | A | G | C |
| S1GA | A10 | G | G | 0 | 0 | A | G | C |
| S3.2 | A10 | G | G | 0 | 0 | A | G | C |
| DNA7.2 | A10 | G | G | 0 | 0 | A | G | C |
| LA2 | A7 | G | G | 0 | 0 | A | G | C |
| | | | Mi/I | ORF- | ORF- | R/H | G/D | 12SS |

| | SNP | | | | | |
|---|---|---|---|---|---|---|
| | SNPG1018 | SNPG1048 | SNPG1056 | SNPG1069 | SNPG1073 | SNPG1079 |
| | R | M | Y | K | R | R |
| AA CHANGE | 1SS | T/P | F/F | Y/D | Y/C | H/R |
| Boleth.1 | A | A | C | T | A | A |
| Boleth.2 | A | A | C | T | A | A |
| P19.1 | A | A | C | T | A | A |
| DNA14.1 | A | A | C | T | A | A |
| P10.1 | A | A | C | T | A | A |
| P1.1 | G | A | C | T | A | A |
| LA1 | G | A | C | T | A | A |
| P1.2 | G | A | C | T | A | A |
| P5.2 | G | A | C | T | A | A |
| AC004204 | G | A | C | T | A | A |
| BX248310 | G | A | C | T | A | A |
| AL84544 | G | A | C | T | A | A |
| S2AG | G | A | C | T | A | A |
| DNA5.1 | G | A | C | T | A | A |
| DNA4.1 | G | A | C | T | A | A |
| DNA7.11 | G | A | C | T | A | A |
| DNA9.1 | G | A | C | T | A | A |
| DNA15.1 | G | A | C | T | A | A |
| DNA15.2 | G | A | C | T | A | A |
| DNA19.1 | G | A | C | T | A | A |
| AL662833 | G | A | C | T | A | A |
| AP000508 | G | A | C | T | A | A |
| S5.2 | G | A | C | T | A | A |
| S4.1 | G | A | C | T | A | A |
| S4.2 | G | A | C | T | A | A |
| DNA8 | G | A | C | T | A | A |
| DNA8.2 | G | A | C | T | A | A |
| DNA12.1 | G | A | C | T | A | A |
| P2.1 | G | A | C | T | A | A |
| P16.1 | G | A | C | T | A | A |
| P5.1 | G | C | C | G | G | G |
| P9.1 | G | C | C | G | G | G |
| P6.2 | G | C | C | G | G | G |
| S1AC | G | C | C | G | G | G |
| S2CA | G | C | C | G | G | G |
| S3.1 | G | C | C | G | G | G |
| DNA1.1 | G | C | C | G | G | G |
| DNA1.2 | G | C | C | G | G | G |
| DNA6.1 | G | C | C | G | G | G |
| DNA9.2 | G | C | C | G | G | G |
| DNA10 | G | C | C | G | G | G |
| DNA10.2 | G | C | C | G | G | G |
| DNA17.1 | G | C | C | G | G | G |
| DNA13.1 | G | C | C | G | G | G |
| P12.1 | G | C | C | G | G | G |
| AL67188 | G | C | C | G | G | G |
| Lucy.1 | G | C | C | G | G | G |
| DNA18.1 | G | C | C | G | G | A |
| DNA18.2 | G | C | C | G | G | A |
| DNA19.2 | G | C | C | G | G | A |
| DNA3 | G | C | C | G | G | A |
| DNA3 | G | C | C | G | G | A |
| P14.1 | G | C | C | G | G | A |
| P4.1 | G | C | C | G | G | A |
| P7.1 | G | C | C | G | G | A |
| DNA11.2 | G | C | C | G | G | G |
| DNA12.2 | G | C | C | G | G | G |
| DNA14.2 | G | C | C | G | G | G |
| DNA16.1 | G | C | C | G | G | G |
| DNA16.2 | G | C | C | G | G | G |
| DNA17.2 | G | C | C | G | G | G |
| DNA2.2 | G | C | C | G | G | G |
| DNA20.1 | G | C | C | G | G | G |
| DNA20.2 | G | C | C | G | G | G |
| DNA4.2 | G | C | C | G | G | G |
| DNA6.2 | G | C | C | G | G | G |
| LucyA22 | G | C | C | G | G | G |
| P12.2 | G | C | C | G | G | G |
| P13.1 | G | C | C | G | G | G |
| P13.2 | G | C | C | G | G | G |
| P14.2 | G | C | C | G | G | G |
| P15.2 | G | C | C | G | G | G |
| P16.2 | G | C | C | G | G | G |
| P17.2 | G | C | C | G | G | G |
| P18.1 | G | C | C | G | G | G |
| P18.2 | G | C | C | G | G | G |
| P19.2 | G | C | C | G | G | G |
| P2.2 | G | C | C | G | G | G |
| P3.1 | G | C | C | G | G | G |
| P3.2 | G | C | C | G | G | G |
| P4.2 | G | C | C | G | G | G |
| P6.1 | G | C | C | G | G | G |
| P7.2 | G | C | C | G | G | G |
| P8.1 | G | C | C | G | G | G |
| P8.2 | G | C | C | G | G | G |
| P9.2 | G | C | C | G | G | G |
| S5.1 | G | C | C | G | G | G |
| DNA11.1 | G | C | C | G | G | A |
| DNA13.2 | G | C | C | G | G | A |
| P10.2 | G | C | C | G | G | A |
| P15.1 | G | C | C | G | G | A |
| P17.1 | G | C | C | G | G | A |
| P11.1 | G | C | C | G | G | A |
| P11.2 | G | C | C | G | G | A |
| DNA5.2 | G | C | C | G | G | A |
| DNA2.1 | G | C | C | G | G | A |
| S1GA | G | C | C | G | G | A |
| S3.2 | G | C | C | G | G | A |
| DNA7.2 | G | C | C | G | G | A |
| LA2 | G | C | T | G | G | G |
| | 1SS | T/P | F/F | Y/D | Y/C | H/R |
| | SNP | | | | | |
| | SNPG1108 | SNPG1154 | SNPG1181 | SNPG1199 | SNPG1236 | SNPG1262 |
| | R | R | R | R | R | Y |
| AA CHANGE | K/R | R/K | K/R | K/R | E/K | M/T |
| Boleth.1 | A | G | A | G | G | C |
| Boleth.2 | A | G | A | G | G | C |
| P19.1 | A | G | A | G | G | C |
| DNA14.1 | A | G | A | G | G | C |
| P10.1 | A | G | A | G | G | C |
| P1.1 | A | G | A | G | G | C |
| LA1 | A | G | A | G | G | T |
| P1.2 | A | G | A | G | G | T |
| P5.2 | A | G | A | G | G | T |
| AC004204 | A | G | A | G | G | T |
| BX248310 | A | G | A | G | G | T |
| AL84544 | A | G | A | G | G | T |
| S2AG | A | G | A | G | G | T |
| DNA5.1 | A | G | A | G | G | T |
| DNA4.1 | A | G | A | G | G | T |
| DNA7.11 | A | G | A | G | G | T |
| DNA9.1 | A | G | A | G | G | T |
| DNA15.1 | A | G | A | G | G | T |
| DNA15.2 | A | G | A | G | G | T |
| DNA19.1 | A | G | A | G | G | T |
| AL662833 | A | G | A | A | G | C |
| AP000508 | A | G | A | A | G | C |
| S5.2 | A | G | A | A | G | C |
| S4.1 | A | G | A | A | G | C |
| S4.2 | A | G | A | A | G | C |
| DNA8 | A | G | A | A | G | C |
| DNA8.2 | A | G | A | A | G | C |
| DNA12.1 | A | G | A | A | G | C |
| P2.1 | A | G | A | A | G | C |
| P16.1 | A | G | A | A | G | C |
| P5.1 | A | G | A | G | G | C |
| P9.1 | A | G | A | G | G | C |
| P6.2 | A | A | A | G | G | C |
| S1AC | A | G | A | G | G | C |
| S2CA | A | G | A | G | G | C |
| S3.1 | A | G | A | G | G | C |
| DNA1.1 | A | G | A | G | G | C |
| DNA1.2 | A | G | A | G | G | C |
| DNA6.1 | A | G | A | G | G | C |
| DNA9.2 | A | G | A | G | G | C |
| DNA10 | A | G | A | G | G | C |
| DNA10.2 | A | G | A | G | G | C |
| DNA17.1 | A | G | A | G | G | C |
| DNA13.1 | A | G | A | G | G | C |
| P12.1 | A | G | A | G | G | C |
| AL67188 | A | G | A | G | G | C |
| Lucy.1 | A | G | A | G | G | C |
| DNA18.1 | A | G | G | G | G | C |
| DNA18.2 | A | G | G | G | G | C |
| DNA19.2 | A | G | G | G | G | C |
| DNA3 | A | G | G | G | G | C |
| DNA3 | A | G | G | G | G | C |
| P14.1 | A | G | G | G | G | C |
| P4.1 | A | G | G | G | G | C |
| P7.1 | A | G | G | G | G | C |
| DNA11.2 | A | G | A | G | G | C |
| DNA12.2 | A | G | A | G | G | C |
| DNA14.2 | A | G | A | G | G | C |
| DNA16.1 | A | G | A | G | G | C |
| DNA16.2 | A | G | A | G | G | C |
| DNA17.2 | A | G | A | G | G | C |
| DNA2.2 | A | G | A | G | G | C |
| DNA20.1 | A | G | A | G | G | C |
| DNA20.2 | A | G | A | G | G | C |
| DNA4.2 | A | G | A | A | G | C |
| DNA6.2 | A | G | A | G | G | C |
| LucyA22 | A | G | A | G | G | C |
| P12.2 | A | G | A | G | G | C |
| P13.1 | A | G | A | G | G | C |
| P13.2 | A | G | A | G | G | C |
| P14.2 | A | G | A | G | G | C |
| P15.2 | A | G | A | G | G | C |
| P16.2 | A | G | A | G | G | C |
| P17.2 | A | G | A | G | G | C |
| P18.1 | A | G | A | G | G | C |
| P18.2 | A | G | A | G | G | C |
| P19.2 | A | G | A | G | G | C |
| P2.2 | A | G | A | G | G | C |
| P3.1 | A | G | A | G | G | C |
| P3.2 | A | G | A | G | G | C |
| P4.2 | A | G | A | G | G | C |
| P6.1 | A | G | A | G | G | C |
| P7.2 | A | G | A | G | G | C |
| P8.1 | A | G | A | G | G | C |
| P8.2 | A | G | A | G | G | C |
| P9.2 | A | G | A | G | G | C |
| S5.1 | A | G | A | G | G | C |
| DNA11.1 | A | G | A | G | A | C |
| DNA13.2 | A | G | A | G | A | C |
| P10.2 | A | G | A | G | A | C |
| P15.1 | A | G | A | G | A | C |
| P17.1 | A | G | A | G | A | C |
| P11.1 | A | G | A | G | A | C |
| P11.2 | A | G | A | G | A | C |
| DNA5.2 | A | G | A | G | A | C |
| DNA2.1 | A | G | A | G | A | C |
| S1GA | A | G | A | G | A | C |
| S3.2 | A | G | A | G | A | C |
| DNA7.2 | A | G | A | G | A | C |
| LA2 | G | G | A | G | A | C |
| | K/R | R/K | K/R | K/R | E/K | M/T |

TABLE 5

| | | SNP | | | | | |
|---|---|---|---|---|---|---|---|
| | | SNPG1263 | SNPG1274 | SNPG1319 | SNPG1334 | SNPG1426 | SNPG1444 |
| | | R | R | K | R | S | K |
| AA CHANGE | alllele | M/T | G/D | S/M | N/S | D/H | D/Y |
| Boleth.1 | A1.1 | G | A | G | A | G | G |
| Boleth.2 | A1.1 | G | A | G | A | G | G |
| P19.1 | A1.1 | G | A | G | A | G | G |
| DNA14.1 | A1.2 | G | A | G | A | G | G |
| P10.1 | A1.2 | G | A | G | A | G | G |
| P1.1 | A11 | G | A | G | A | G | G |
| LA1 | A2 | G | G | G | A | G | G |
| P1.2 | A3 | G | G | G | A | G | G |
| P5.2 | A3 | G | G | G | A | G | G |
| AC004204 | A3 | G | G | G | A | G | G |
| BX248310 | A3 | G | G | G | A | G | G |
| AL84544 | A3 | G | G | G | A | G | G |
| S2AG | A3 | G | G | G | A | G | G |
| DNA5.1 | A3 | G | G | G | A | G | G |
| DNA4.1 | A3 | G | G | G | A | G | G |
| DNA7.11 | A3 | G | G | G | A | G | G |
| DNA9.1 | A3 | G | G | G | A | G | G |
| DNA15.1 | A3 | G | G | G | A | G | G |
| DNA15.2 | A3 | G | G | G | A | G | G |
| DNA19.1 | A3 | G | G | G | A | G | G |
| AL662833 | A4.1 | G | G | G | A | G | G |
| AP000508 | A4.1 | G | G | G | A | G | G |
| S5.2 | A4.1 | G | G | G | A | G | G |
| S4.1 | A4.2 | G | G | G | A | G | G |
| S4.2 | A4.2 | G | G | G | A | G | G |
| DNA8 | A4.1 | G | G | G | A | G | G |
| DNA8.2 | A4.1 | G | G | G | A | G | G |
| DNA12.1 | A4.1 | G | G | G | A | G | G |
| P2.1 | A4.1 | G | G | G | A | G | G |
| P16.1 | A4.1 | G | G | G | A | G | G |
| P5.1 | A5.2 | A | G | G | A | G | G |
| P9.1 | A5.1 | G | G | G | A | G | G |
| P6.2 | A12 | G | G | G | A | G | G |
| S1AC | A6 | G | G | G | A | G | G |
| S2CA | A6 | G | G | G | A | G | G |
| S3.1 | A6 | G | G | G | A | G | G |
| DNA1.1 | A6 | G | G | G | A | G | G |
| DNA1.2 | A6 | G | G | G | A | G | G |
| DNA6.1 | A6 | G | G | G | A | G | G |
| DNA9.2 | A6 | G | G | G | A | G | G |
| DNA10 | A6 | G | G | G | A | G | G |
| DNA10.2 | A6 | G | G | G | A | G | G |
| DNA17.1 | A6 | G | G | G | A | G | G |
| DNA13.1 | A6 | G | G | G | A | G | G |
| P12.1 | A6 | G | G | G | A | G | G |
| AL67188 | A6 | G | G | G | A | G | G |
| Lucy.1 | A6 | G | G | G | A | G | G |
| DNA18.1 | A8 | G | G | G | A | C | G |
| DNA18.2 | A8 | G | G | G | A | C | G |
| DNA19.2 | A8 | G | G | G | A | C | G |
| DNA3 | A8 | G | G | G | A | C | G |
| DNA3 | A8 | G | G | G | A | C | G |
| P14.1 | A8 | G | G | G | A | C | G |
| P4.1 | A8 | G | G | G | A | C | G |
| P7.1 | A8 | G | G | G | A | C | G |
| DNA11.2 | A9.2 | G | G | G | A | C | T |
| DNA12.2 | A9.1 | G | A | G | A | C | T |
| DNA14.2 | A9.1 | G | A | G | A | C | T |
| DNA16.1 | A9.1 | G | A | G | A | C | T |
| DNA16.2 | A9.1 | G | A | G | A | C | T |
| DNA17.2 | A9.1 | G | A | G | A | C | T |
| DNA2.2 | A9.3 | G | A | T | A | C | T |
| DNA20.1 | A9.1 | G | A | G | A | C | T |
| DNA20.2 | A9.1 | G | A | G | A | C | T |
| DNA4.2 | A9.1 | G | A | G | A | C | T |
| DNA6.2 | A9.1 | G | A | G | A | C | T |
| LucyA22 | A9.1 | G | A | G | A | C | T |
| P12.2 | A9.1 | G | A | G | A | C | T |
| P13.1 | A9.1 | G | A | G | A | C | T |
| P13.2 | A9.1 | G | A | G | A | C | T |
| P14.2 | A9.1 | G | A | G | A | C | T |
| P15.2 | A9.1 | G | A | G | A | C | T |
| P16.2 | A9.1 | G | A | G | A | C | T |
| P17.2 | A9.1 | G | A | G | A | C | T |
| P18.1 | A9.1 | G | A | G | A | C | T |
| P18.2 | A9.2 | G | G | G | A | C | T |
| P19.2 | A9.2 | G | G | G | A | C | T |
| P2.2 | A9.1 | G | A | G | A | C | T |
| P3.1 | A9.1 | G | A | G | A | C | T |
| P3.2 | A9.2 | G | G | G | A | C | T |
| P4.2 | A9.1 | G | A | G | A | C | T |
| P6.1 | A9.1 | G | A | G | A | C | T |
| P7.2 | A9.3 | G | A | T | A | C | T |
| P8.1 | A9.1 | G | A | G | A | C | T |
| P8.2 | A9.1 | G | A | G | A | C | T |
| P9.2 | A9.2 | G | G | G | A | C | T |
| S5.1 | A9.1 | G | A | G | A | C | T |
| DNA11.1 | A10 | G | G | G | A | C | T |
| DNA13.2 | A10 | G | G | G | A | C | T |
| P10.2 | A10 | G | G | G | A | C | T |
| P15.1 | A10 | G | G | G | A | C | T |
| P17.1 | A10 | G | G | G | A | C | T |
| P11.1 | A10 | G | G | G | A | C | T |
| P11.2 | A10 | G | G | G | A | C | T |
| DNA5.2 | A10 | G | G | G | A | C | T |
| DNA2.1 | A10 | G | G | G | A | C | T |
| S1GA | A10 | G | G | G | A | C | T |
| S3.2 | A10 | G | G | G | A | C | T |
| DNA7.2 | A10 | G | G | G | A | C | T |
| LA2 | A7 | G | G | G | G | C | T |

| | SNP | | | | | |
|---|---|---|---|---|---|---|
| | SNPG1466 | SNPG1487 | SNPG1489 | SNPG1505 | SNPG1508 | SNPG1518 |
| | Y | R | R | K | Y | Y |
| AA CHANGE | L/S | D/G | K/E | G/V | V/A | S/S |
| Boleth.1 | T | G | A | G | T | C |
| Boleth.2 | T | G | A | G | T | C |
| P19.1 | T | G | A | G | T | C |
| DNA14.1 | T | G | A | G | T | C |
| P10.1 | T | G | A | G | T | C |
| P1.1 | T | G | A | G | T | C |
| LA1 | T | A | A | G | C | C |
| P1.2 | T | A | A | G | T | C |
| P5.2 | T | A | A | G | T | C |
| AC004204 | T | A | A | G | T | C |
| BX248310 | T | A | A | G | T | C |
| AL84544 | T | A | A | G | T | C |
| S2AG | T | A | A | G | T | C |
| DNA5.1 | T | A | A | G | T | C |
| DNA4.1 | T | A | A | G | T | C |
| DNA7.11 | T | A | A | G | T | C |
| DNA9.1 | T | A | A | G | T | C |
| DNA15.1 | T | A | A | G | T | C |
| DNA15.2 | T | A | A | G | T | C |
| DNA19.1 | T | A | A | G | T | C |
| AL662833 | T | A | G | T | T | T |
| AP000508 | T | A | G | T | T | T |
| S5.2 | T | A | G | T | T | T |
| S4.1 | T | A | G | T | T | T |
| S4.2 | T | A | G | T | T | T |
| DNA8 | T | A | G | T | T | T |
| DNA8.2 | T | A | G | T | T | T |
| DNA12.1 | T | A | G | T | T | T |
| P2.1 | T | A | G | T | T | T |
| P16.1 | T | A | G | T | T | T |
| P5.1 | T | A | G | G | T | T |
| P9.1 | T | A | G | G | T | T |
| P6.2 | T | G | A | G | T | T |
| S1AC | T | A | G | G | T | C |
| S2CA | T | A | G | G | T | C |
| S3.1 | T | A | G | G | T | C |
| DNA1.1 | T | A | G | G | T | C |
| DNA1.2 | T | A | G | G | T | C |
| DNA6.1 | T | A | G | G | T | C |
| DNA9.2 | T | A | G | G | T | C |
| DNA10 | T | A | G | G | T | C |
| DNA10.2 | T | A | G | G | T | C |
| DNA17.1 | T | A | G | G | T | C |
| DNA13.1 | T | A | G | G | T | C |
| P12.1 | T | A | G | G | T | C |
| AL67188 | T | A | G | G | T | C |
| Lucy.1 | T | A | G | G | T | C |
| DNA18.1 | T | G | A | G | T | C |
| DNA18.2 | T | G | A | G | T | C |
| DNA19.2 | T | G | A | G | T | C |
| DNA3 | T | G | A | G | T | C |
| DNA3 | T | G | A | G | T | C |
| P14.1 | T | G | A | G | T | C |
| P4.1 | T | G | A | G | T | C |
| P7.1 | T | G | A | G | T | C |
| DNA11.2 | T | A | A | G | T | C |
| DNA12.2 | T | A | A | G | T | C |
| DNA14.2 | T | A | A | G | T | C |
| DNA16.1 | T | A | A | G | T | C |
| DNA16.2 | T | A | A | G | T | C |
| DNA17.2 | T | A | A | G | T | C |
| DNA2.2 | T | A | A | G | T | C |
| DNA20.1 | T | A | A | G | T | C |
| DNA20.2 | T | A | A | G | T | C |
| DNA4.2 | T | A | A | G | T | C |
| DNA6.2 | T | A | A | G | T | C |
| LucyA22 | T | A | A | G | T | C |
| P12.2 | T | A | A | G | T | C |
| P13.1 | T | A | A | G | T | C |
| P13.2 | T | A | A | G | T | C |
| P14.2 | T | A | A | G | T | C |
| P15.2 | T | A | A | G | T | C |
| P16.2 | T | A | A | G | T | C |
| P17.2 | T | A | A | G | T | C |
| P18.1 | T | A | A | G | T | C |
| P18.2 | T | A | A | G | T | C |
| P19.2 | T | A | A | G | T | C |
| P2.2 | T | A | A | G | T | C |
| P3.1 | T | A | A | G | T | C |
| P3.2 | T | A | A | G | T | C |
| P4.2 | T | A | A | G | T | C |
| P6.1 | T | A | A | G | T | C |
| P7.2 | T | A | A | G | T | C |
| P8.1 | T | A | A | G | T | C |
| P8.2 | T | A | A | G | T | C |
| P9.2 | T | A | A | G | T | C |
| S5.1 | T | A | A | G | T | C |
| DNA11.1 | T | A | A | G | T | C |
| DNA13.2 | T | A | A | G | T | C |
| P10.2 | T | A | A | G | T | C |
| P15.1 | T | A | A | G | T | C |
| P17.1 | T | A | A | G | T | C |
| P11.1 | T | A | A | G | T | C |
| P11.2 | T | A | A | G | T | C |
| DNA5.2 | T | A | A | G | T | C |
| DNA2.1 | T | A | A | G | T | C |
| S1GA | T | A | A | G | T | C |
| S3.2 | T | A | A | G | T | C |
| DNA7.2 | T | A | A | G | T | C |
| LA2 | C | A | A | G | T | C |
| | SNP | | | | | |
| | SNPG1521 | SNPG1543 | SNPG1554 | SNPG1630 | SNPG1638 | SNPG1680 |
| | K | R | Y | R | W | R |
| AA CHANGE | W/C | D/N | D/D | G/R | T/T | 5SS |
| Boleth.1 | G | G | T | G | A | A |
| Boleth.2 | G | G | T | G | A | A |
| P19.1 | G | G | T | G | A | A |
| DNA14.1 | G | G | T | G | A | A |
| P10.1 | G | G | T | G | A | A |
| P1.1 | G | G | T | G | A | A |
| LA1 | G | G | T | G | A | A |
| P1.2 | G | G | T | G | A | A |
| P5.2 | G | G | T | G | A | A |
| AC004204 | G | G | T | G | A | A |
| BX248310 | G | G | T | G | A | A |
| AL84544 | G | G | T | G | A | A |
| S2AG | G | G | T | G | A | A |
| DNA5.1 | G | G | T | G | A | A |
| DNA4.1 | G | G | T | G | A | A |
| DNA7.11 | G | G | T | G | A | A |
| DNA9.1 | G | G | T | G | A | A |
| DNA15.1 | G | G | T | G | A | A |
| DNA15.2 | G | G | T | G | A | A |
| DNA19.1 | G | G | T | G | A | A |
| AL662833 | G | A | C | A | T | G |
| AP000508 | G | A | C | A | T | G |
| S5.2 | G | A | C | A | T | G |
| S4.1 | G | A | C | A | T | G |
| S4.2 | G | A | C | A | T | G |
| DNA8 | G | A | C | A | T | G |
| DNA8.2 | G | A | C | A | T | G |
| DNA12.1 | G | A | C | A | T | G |
| P2.1 | G | A | C | A | T | G |
| P16.1 | G | A | C | A | T | G |
| P5.1 | T | A | C | G | A | A |
| P9.1 | T | A | C | G | A | A |
| P6.2 | G | A | C | G | A | A |
| S1AC | G | G | C | A | T | G |
| S2CA | G | G | C | A | T | G |
| S3.1 | G | G | C | A | T | G |
| DNA1.1 | G | G | C | A | T | G |
| DNA1.2 | G | G | C | A | T | G |
| DNA6.1 | G | G | C | A | T | G |
| DNA9.2 | G | G | C | A | T | G |
| DNA10 | G | G | C | A | T | G |
| DNA10.2 | G | G | C | A | T | G |
| DNA17.1 | G | G | C | A | T | G |
| DNA13.1 | G | G | C | A | T | G |
| P12.1 | G | G | C | A | T | G |
| AL67188 | G | G | C | A | T | G |
| Lucy.1 | G | G | C | A | T | G |
| DNA18.1 | G | G | T | G | A | A |
| DNA18.2 | G | G | T | G | A | A |
| DNA19.2 | G | G | T | G | A | A |
| DNA3 | G | G | T | G | A | A |
| DNA3 | G | G | T | G | A | A |
| P14.1 | G | G | T | G | A | A |
| P4.1 | G | G | T | G | A | A |
| P7.1 | G | G | T | G | A | A |
| DNA11.2 | G | G | T | G | A | A |
| DNA12.2 | G | G | T | G | A | A |
| DNA14.2 | G | G | T | G | A | A |
| DNA16.1 | G | G | T | G | A | A |
| DNA16.2 | G | G | T | G | A | A |
| DNA17.2 | G | G | T | G | A | A |
| DNA2.2 | G | G | T | G | A | A |
| DNA20.1 | G | G | T | G | A | A |
| DNA20.2 | G | G | T | G | A | A |
| DNA4.2 | G | G | T | G | A | A |
| DNA6.2 | G | G | T | G | A | A |
| LucyA22 | G | G | T | G | A | A |
| P12.2 | G | G | T | G | A | A |
| P13.1 | G | G | T | G | A | A |
| P13.2 | G | G | T | G | A | A |
| P14.2 | G | G | T | G | A | A |
| P15.2 | G | G | T | G | A | A |
| P16.2 | G | G | T | G | A | A |
| P17.2 | G | G | T | G | A | A |
| P18.1 | G | G | T | G | A | A |
| P18.2 | G | G | T | G | A | A |
| P19.2 | G | G | T | G | A | A |
| P2.2 | G | G | T | G | A | A |
| P3.1 | G | G | T | G | A | A |
| P3.2 | G | G | T | G | A | A |
| P4.2 | G | G | T | G | A | A |
| P6.1 | G | G | T | G | A | A |
| P7.2 | G | G | T | G | A | A |
| P8.1 | G | G | T | G | A | A |
| P8.2 | G | G | T | G | A | A |
| P9.2 | G | G | T | G | A | A |
| S5.1 | G | G | T | G | A | A |
| DNA11.1 | G | G | T | G | A | A |
| DNA13.2 | G | G | T | G | A | A |
| P10.2 | G | G | T | G | A | A |
| P15.1 | G | G | T | G | A | A |
| P17.1 | G | G | T | G | A | A |
| P11.1 | G | G | T | G | A | A |
| P11.2 | G | G | T | G | A | A |
| DNA5.2 | G | G | T | G | A | A |
| DNA2.1 | G | G | T | G | A | A |
| S1GA | G | G | T | G | A | A |
| S3.2 | G | G | T | G | A | A |
| DNA7.2 | G | G | T | G | A | A |
| LA2 | G | G | T | G | A | A |

TABLE 6

| | | SNP | | | | | | |
|---|---|---|---|---|---|---|---|---|
| | | SNPG1687 | SNPG1707 | SNPG1728 | SNPG1742 | SNPG1810 | SNPG1813 | SNPG1841 |
| | | M | dC | R | Y | Y | M | Y |
| AA CHANGE | alllele | int2 | ORF | Y/C | P/L | V/A | I/L | I/T |
| Boleth.1 | A1.1 | C | C | A | C | T | A | T |
| Boleth.2 | A1.1 | C | C | A | C | T | A | T |
| P19.1 | A1.1 | C | C | A | C | T | A | T |
| DNA14.1 | A1.2 | C | C | A | C | T | A | T |
| P10.1 | A1.2 | C | C | A | C | T | A | T |
| P1.1 | A11 | C | C | A | C | T | A | T |
| LA1 | A2 | C | C | A | C | T | A | T |
| P1.2 | A3 | C | C | A | C | T | A | T |
| P5.2 | A3 | C | C | A | C | T | A | T |
| AC004204 | A3 | C | C | A | C | T | A | T |
| BX248310 | A3 | C | C | A | C | T | A | T |
| AL84544 | A3 | C | C | A | C | T | A | T |
| S2AG | A3 | C | C | A | C | T | A | T |
| DNA5.1 | A3 | C | C | A | C | T | A | T |
| DNA4.1 | A3 | C | C | A | C | T | A | T |
| DNA7.11 | A3 | C | C | A | C | T | A | T |
| DNA9.1 | A3 | C | C | A | C | T | A | T |
| DNA15.1 | A3 | C | C | A | C | T | A | T |
| DNA15.2 | A3 | C | C | A | C | T | A | T |
| DNA19.1 | A3 | C | C | A | C | T | A | T |
| AL662833 | A4.1 | C | 0 | A | C | T | A | T |
| AP000508 | A4.1 | C | 0 | A | C | T | A | T |
| S5.2 | A4.1 | C | 0 | A | C | T | A | T |
| S4.1 | A4.2 | C | 0 | A | C | T | A | T |
| S4.2 | A4.2 | C | 0 | A | C | T | A | T |
| DNA8 | A4.1 | C | 0 | A | C | T | A | T |
| DNA8.2 | A4.1 | C | 0 | A | C | T | A | T |
| DNA12.1 | A4.1 | C | 0 | A | C | T | A | T |
| P2.1 | A4.1 | C | 0 | A | C | T | A | T |
| P16.1 | A4.1 | C | 0 | A | C | T | A | T |
| P5.1 | A5.2 | A | C | A | C | T | A | T |
| P9.1 | A5.1 | A | C | A | C | T | A | T |
| P6.2 | A12 | A | C | A | C | T | A | T |
| S1AC | A6 | C | 0 | G | C | T | A | C |
| S2CA | A6 | C | 0 | G | C | T | A | C |
| S3.1 | A6 | C | 0 | G | C | T | A | C |
| DNA1.1 | A6 | C | 0 | G | C | T | A | C |
| DNA1.2 | A6 | C | 0 | G | C | T | A | C |
| DNA6.1 | A6 | C | 0 | G | C | T | A | C |
| DNA9.2 | A6 | C | 0 | G | C | T | A | C |
| DNA10 | A6 | C | 0 | G | C | T | A | C |
| DNA10.2 | A6 | C | 0 | G | C | T | A | C |
| DNA17.1 | A6 | C | 0 | G | C | T | A | C |
| DNA13.1 | A6 | C | 0 | G | C | T | A | C |
| P12.1 | A6 | C | 0 | G | C | T | A | C |
| AL67188 | A6 | C | 0 | G | C | T | A | C |
| Lucy.1 | A6 | C | 0 | G | C | T | A | C |
| DNA18.1 | A8 | C | C | A | C | T | C | T |
| DNA18.2 | A8 | C | C | A | C | T | C | T |
| DNA19.2 | A8 | C | C | A | C | T | C | T |
| DNA3 | A8 | C | C | A | C | T | C | T |
| DNA3 | A8 | C | C | A | C | T | C | T |
| P14.1 | A8 | C | C | A | C | T | C | T |
| P4.1 | A8 | C | C | A | C | T | C | T |
| P7.1 | A8 | C | C | A | C | T | C | T |
| DNA11.2 | A9.2 | C | C | A | T | T | A | T |
| DNA12.2 | A9.1 | C | C | A | T | T | A | T |
| DNA14.2 | A9.1 | C | C | A | T | T | A | T |
| DNA16.1 | A9.1 | C | C | A | T | T | A | T |
| DNA16.2 | A9.1 | C | C | A | T | T | A | T |
| DNA17.2 | A9.1 | C | C | A | T | T | A | T |
| DNA2.2 | A9.3 | C | C | A | T | T | A | T |
| DNA20.1 | A9.1 | C | C | A | T | T | A | T |
| DNA20.2 | A9.1 | C | C | A | T | T | A | T |
| DNA4.2 | A9.1 | C | C | A | T | T | A | T |
| DNA6.2 | A9.1 | C | C | A | T | T | A | T |
| LucyA22 | A9.1 | C | C | A | T | T | A | T |
| P12.2 | A9.1 | C | C | A | T | T | A | T |
| P13.1 | A9.1 | C | C | A | T | T | A | T |
| P13.2 | A9.1 | C | C | A | T | T | A | T |
| P14.2 | A9.1 | C | C | A | T | T | A | T |
| P15.2 | A9.1 | C | C | A | T | T | A | T |
| P16.2 | A9.1 | C | C | A | T | T | A | T |
| P17.2 | A9.1 | C | C | A | T | T | A | T |
| P18.1 | A9.1 | C | C | A | T | T | A | T |
| P18.2 | A9.2 | C | C | A | T | T | A | T |
| P19.2 | A9.2 | C | C | A | T | T | A | T |
| P2.2 | A9.1 | C | C | A | T | T | A | T |
| P3.1 | A9.1 | C | C | A | T | T | A | T |
| P3.2 | A9.2 | C | C | A | T | T | A | T |
| P4.2 | A9.1 | C | C | A | T | T | A | T |
| P6.1 | A9.1 | C | C | A | T | T | A | T |
| P7.2 | A9.3 | C | C | A | T | T | A | T |
| P8.1 | A9.1 | C | C | A | T | T | A | T |
| P8.2 | A9.1 | C | C | A | T | T | A | T |
| P9.2 | A9.2 | C | C | A | T | T | A | T |
| S5.1 | A9.1 | C | C | A | T | T | A | T |
| DNA11.1 | A10 | C | C | A | C | C | A | T |
| DNA13.2 | A10 | C | C | A | C | C | A | T |
| P10.2 | A10 | C | C | A | C | C | A | T |
| P15.1 | A10 | C | C | A | C | C | A | T |
| P17.1 | A10 | C | C | A | C | C | A | T |
| P11.1 | A10 | C | C | A | C | C | A | T |
| P11.2 | A10 | C | C | A | C | C | A | T |
| DNA5.2 | A10 | C | C | A | C | C | A | T |
| DNA2.1 | A10 | C | C | A | C | C | A | T |
| S1GA | A10 | C | C | A | C | C | A | T |
| S3.2 | A10 | C | C | A | C | C | A | T |
| DNA7.2 | A10 | C | C | A | C | C | A | T |
| LA2 | A7 | C | C | A | C | C | A | T |

| | SNP | | | | | | |
|---|---|---|---|---|---|---|---|
| | SNPG1874 | SNPG1875 | SNPG1890 | SNPG1895 | SNPG1907 | SNPG1909 | SNPG1921 |
| | S | R | M | R | R | Y | M |
| AA CHANGE | R/P | R/R | T/T | K/R | K/R | H/R | I/I |
| Boleth.1 | G | A | C | G | G | C | A |
| Boleth.2 | G | A | C | G | G | C | A |
| P19.1 | G | A | C | G | G | C | A |
| DNA14.1 | G | A | C | G | G | C | A |
| P10.1 | G | A | C | G | G | C | A |
| P1.1 | G | A | C | G | G | C | A |
| LA1 | G | G | C | A | G | C | A |
| P1.2 | G | A | C | A | G | C | A |
| P5.2 | G | A | C | A | G | C | A |
| AC004204 | G | A | C | A | G | C | A |
| BX248310 | G | A | C | A | G | C | A |
| AL84544 | G | A | C | A | G | C | A |
| S2AG | G | A | C | A | G | C | A |
| DNA5.1 | G | A | C | A | G | C | A |
| DNA4.1 | G | A | C | A | G | C | A |
| DNA7.11 | G | A | C | A | G | C | A |
| DNA9.1 | G | A | C | A | G | C | A |
| DNA15.1 | G | A | C | A | G | C | A |
| DNA15.2 | G | A | C | A | G | C | A |
| DNA19.1 | G | A | C | A | G | C | A |
| AL662833 | G | A | C | G | G | C | A |
| AP000508 | G | A | C | G | G | C | A |
| S5.2 | G | A | C | G | G | C | A |
| S4.1 | G | A | C | G | G | C | A |
| S4.2 | G | A | C | G | G | C | A |
| DNA8 | G | A | C | G | G | C | A |
| DNA8.2 | G | A | C | G | G | C | A |
| DNA12.1 | G | A | C | G | G | C | A |
| P2.1 | G | A | C | G | G | C | A |
| P16.1 | G | A | C | G | G | C | A |
| P5.1 | G | A | C | G | G | C | A |
| P9.1 | G | A | C | G | G | C | A |
| P6.2 | G | A | C | G | G | C | A |
| S1AC | C | A | A | G | A | T | C |
| S2CA | C | A | A | G | A | T | C |
| S3.1 | C | A | A | G | A | T | C |
| DNA1.1 | C | A | A | G | A | T | C |
| DNA1.2 | C | A | A | G | A | T | C |
| DNA6.1 | C | A | A | G | A | T | C |
| DNA9.2 | C | A | A | G | A | T | C |
| DNA10 | C | C | A | G | A | T | C |
| DNA10.2 | C | C | A | G | A | T | C |
| DNA17.1 | C | C | A | G | A | T | C |
| DNA13.1 | C | A | A | G | A | T | C |
| P12.1 | C | A | A | G | A | T | C |
| AL67188 | C | A | A | G | A | T | C |
| Lucy.1 | C | A | A | G | A | T | C |
| DNA18.1 | G | A | C | G | G | C | A |
| DNA18.2 | G | A | C | G | G | C | A |
| DNA19.2 | G | A | C | G | G | C | A |
| DNA3 | G | A | C | G | G | C | A |
| DNA3 | G | A | C | G | G | C | A |
| P14.1 | G | A | C | G | G | C | A |
| P4.1 | G | A | C | G | G | C | A |
| P7.1 | G | A | C | G | G | C | A |
| DNA11.2 | G | A | C | G | G | C | A |
| DNA12.2 | G | A | C | G | G | C | A |
| DNA14.2 | G | A | C | G | G | C | A |
| DNA16.1 | G | A | C | G | G | C | A |
| DNA16.2 | G | A | C | G | G | C | A |
| DNA17.2 | G | A | C | G | G | C | A |
| DNA2.2 | G | A | C | G | G | C | A |
| DNA20.1 | G | A | C | G | G | C | A |
| DNA20.2 | G | A | C | G | G | C | A |
| DNA4.2 | G | A | C | G | G | C | A |
| DNA6.2 | G | A | C | G | G | C | A |
| LucyA22 | G | A | C | G | G | C | A |
| P12.2 | G | A | C | G | G | C | A |
| P13.1 | G | A | C | G | G | C | A |
| P13.2 | G | A | C | G | G | C | A |
| P14.2 | G | A | C | G | G | C | A |
| P15.2 | G | A | C | G | G | C | A |
| P16.2 | G | A | C | G | G | C | A |
| P17.2 | G | A | C | G | G | C | A |
| P18.1 | G | A | C | G | G | C | A |
| P18.2 | G | A | C | G | G | C | A |
| P19.2 | G | A | C | G | G | C | A |
| P2.2 | G | A | C | G | G | C | A |
| P3.1 | G | A | C | G | G | C | A |
| P3.2 | G | A | C | G | G | C | A |
| P4.2 | G | A | C | G | G | C | A |
| P6.1 | G | A | C | G | G | C | A |
| P7.2 | G | A | C | G | G | C | A |
| P8.1 | G | A | C | G | G | C | A |
| P8.2 | G | A | C | G | G | C | A |
| P9.2 | G | A | C | G | G | C | A |
| S5.1 | G | A | C | G | G | C | A |
| DNA11.1 | C | A | C | G | G | C | A |
| DNA13.2 | C | A | C | G | G | C | A |
| P10.2 | C | A | C | G | G | C | A |
| P15.1 | C | A | C | G | G | C | A |
| P17.1 | C | A | C | G | G | C | A |
| P11.1 | C | A | C | G | G | C | A |
| P11.2 | C | A | C | G | G | C | A |
| DNA5.2 | C | A | C | G | G | C | A |
| DNA2.1 | C | A | C | G | G | C | A |
| S1GA | C | A | C | G | G | C | A |
| S3.2 | C | A | C | G | G | C | A |
| DNA7.2 | C | A | C | G | G | C | A |
| LA2 | C | A | C | G | G | C | A |

| | SNP | | | | | |
|---|---|---|---|---|---|---|
| | SNPG1922 | SNPG1957 | SNPG1959 | SNPG1976 | SNPG1992 | SNPG1993 |
| | R | R | R | Y | Y | Y |
| AA CHANGE | S/A | T/A | T/T | S/P | I/T | S/L |
| Boleth.1 | A | A | A | T | T | C |
| Boleth.2 | A | A | A | T | T | C |
| P19.1 | A | A | A | T | T | C |
| DNA14.1 | A | A | A | T | T | T |
| P10.1 | A | A | A | T | T | T |
| P1.1 | G | A | A | T | T | T |
| LA1 | A | A | A | T | T | C |
| P1.2 | A | A | A | T | T | C |
| P5.2 | A | A | A | T | T | C |
| AC004204 | A | A | A | T | T | C |
| BX248310 | A | A | A | T | T | C |
| AL84544 | A | A | A | T | T | C |
| S2AG | A | A | A | T | T | C |
| DNA5.1 | A | A | A | T | T | C |
| DNA4.1 | A | A | A | T | T | C |
| DNA7.11 | A | A | A | T | T | C |
| DNA9.1 | A | A | A | T | T | C |
| DNA15.1 | A | A | A | T | T | C |
| DNA15.2 | A | A | A | T | T | C |
| DNA19.1 | A | A | A | T | T | C |
| AL662833 | A | A | A | T | T | C |
| AP000508 | A | A | A | T | T | C |
| S5.2 | A | A | A | T | T | C |
| S4.1 | A | A | A | T | T | C |
| S4.2 | A | A | A | T | T | C |
| DNA8 | A | A | A | T | T | C |
| DNA8.2 | A | A | A | T | T | C |
| DNA12.1 | A | A | A | T | T | C |
| P2.1 | A | A | A | T | T | C |
| P16.1 | A | A | A | T | T | C |
| P5.1 | A | A | A | T | T | C |
| P9.1 | A | A | A | T | T | C |
| P6.2 | A | A | A | T | T | C |
| S1AC | A | A | G | T | C | C |
| S2CA | A | A | G | T | C | C |
| S3.1 | A | A | G | T | C | C |
| DNA1.1 | A | A | G | T | C | C |
| DNA1.2 | A | A | G | T | C | C |
| DNA6.1 | A | A | G | T | C | C |
| DNA9.2 | A | A | G | T | C | C |
| DNA10 | A | A | G | T | C | C |
| DNA10.2 | A | A | G | T | C | C |
| DNA17.1 | A | A | G | T | C | C |
| DNA13.1 | A | A | G | T | C | C |
| P12.1 | A | A | G | T | C | C |
| AL67188 | A | A | G | T | C | C |
| Lucy.1 | A | A | G | T | C | C |
| DNA18.1 | A | A | A | T | T | C |
| DNA18.2 | A | A | A | T | T | C |
| DNA19.2 | A | A | A | T | T | C |
| DNA3 | A | A | A | T | T | C |
| DNA3 | A | A | A | T | T | C |
| P14.1 | A | A | A | T | T | C |
| P4.1 | A | A | A | T | T | C |
| P7.1 | A | A | A | T | T | C |
| DNA11.2 | A | A | A | T | T | C |
| DNA12.2 | A | A | A | T | T | C |
| DNA14.2 | A | A | A | T | T | C |
| DNA16.1 | A | A | A | T | T | C |
| DNA16.2 | A | A | A | T | T | C |
| DNA17.2 | A | A | A | T | T | C |
| DNA2.2 | A | A | A | T | T | C |
| DNA20.1 | A | A | A | T | T | C |
| DNA20.2 | A | A | A | T | T | C |
| DNA4.2 | A | A | A | T | T | C |
| DNA6.2 | A | A | A | T | T | C |
| LucyA22 | A | A | A | T | T | C |
| P12.2 | A | A | A | T | T | C |
| P13.1 | A | A | A | T | T | C |
| P13.2 | A | A | A | T | T | C |
| P14.2 | A | A | A | T | T | C |
| P15.2 | A | A | A | T | T | C |
| P16.2 | A | A | A | T | T | C |
| P17.2 | A | A | A | T | T | C |
| P18.1 | A | A | A | T | T | C |
| P18.2 | A | A | A | T | T | C |
| P19.2 | A | A | A | T | T | C |
| P2.2 | A | A | A | T | T | C |
| P3.1 | A | A | A | T | T | C |
| P3.2 | A | A | A | T | T | C |
| P4.2 | A | A | A | T | T | C |
| P6.1 | A | A | A | T | T | C |
| P7.2 | A | A | A | T | T | C |
| P8.1 | A | A | A | T | T | C |
| P8.2 | A | A | A | T | T | C |
| P9.2 | A | A | A | T | T | C |
| S5.1 | A | A | A | T | T | C |
| DNA11.1 | A | G | A | T | C | C |
| DNA13.2 | A | G | A | T | C | C |
| P10.2 | A | G | A | T | C | C |
| P15.1 | A | G | A | T | C | C |
| P17.1 | A | G | A | T | C | C |
| P11.1 | A | G | A | T | C | C |
| P11.2 | A | G | A | T | C | C |
| DNA5.2 | A | G | A | T | C | C |
| DNA2.1 | A | G | A | T | C | C |
| S1GA | A | G | A | T | C | C |
| S3.2 | A | G | A | T | C | C |
| DNA7.2 | A | G | A | T | C | C |
| LA2 | A | G | A | C | C | C |

TABLE 7

| | | SNP | | | | | |
|---|---|---|---|---|---|---|---|
| | | SNPG2096 | SNPG2135 | SNPG2180 | SNPG2192 | SNPG2230 | SNPG2275 |
| | | S | R | R | R | S | Y |
| AA CHANGE | alllele | P/S | E/K | H/R | S/N | A/P | Q/* |
| Boleth.1 | A1.1 | C | G | G | G | G | C |
| Boleth.2 | A1.1 | C | G | G | G | G | C |
| P19.1 | A1.1 | C | G | G | G | G | C |
| DNA14.1 | A1.2 | C | G | G | G | G | C |
| P10.1 | A1.2 | C | G | G | G | G | C |
| P1.1 | A11 | C | G | G | G | G | C |
| LA1 | A2 | C | G | A | G | G | C |
| P1.2 | A3 | C | G | A | G | G | C |
| P5.2 | A3 | C | G | A | G | G | C |
| AC004204 | A3 | C | G | A | G | G | C |
| BX248310 | A3 | C | G | A | G | G | C |
| AL84544 | A3 | C | G | A | G | G | C |
| S2AG | A3 | C | G | A | G | G | C |
| DNA5.1 | A3 | C | G | A | G | G | C |
| DNA4.1 | A3 | C | G | A | G | G | C |
| DNA7.11 | A3 | C | G | A | G | G | C |
| DNA9.1 | A3 | C | G | A | G | G | C |
| DNA15.1 | A3 | C | G | A | G | G | C |
| DNA15.2 | A3 | C | G | A | G | G | C |
| DNA19.1 | A3 | C | G | A | G | G | C |
| AL662833 | A4.1 | C | G | G | G | G | C |
| AP000508 | A4.1 | C | G | G | G | G | C |
| S5.2 | A4.1 | C | G | G | G | G | C |
| S4.1 | A4.2 | C | G | G | G | G | C |
| S4.2 | A4.2 | C | G | G | G | G | C |
| DNA8 | A4.1 | C | G | G | G | G | C |
| DNA8.2 | A4.1 | C | G | G | G | G | C |
| DNA12.1 | A4.1 | C | G | G | G | G | C |
| P2.1 | A4.1 | C | G | G | G | G | C |
| P16.1 | A4.1 | C | G | G | G | G | C |
| P5.1 | A5.2 | C | G | G | G | G | C |
| P9.1 | A5.1 | C | G | G | G | G | C |
| P6.2 | A12 | C | G | G | G | G | C |
| S1AC | A6 | C | G | G | G | G | T |
| S2CA | A6 | C | G | G | G | G | T |
| S3.1 | A6 | C | G | G | G | G | T |
| DNA1.1 | A6 | C | G | G | G | G | T |
| DNA1.2 | A6 | C | G | G | G | G | T |
| DNA6.1 | A6 | C | G | G | G | G | T |
| DNA9.2 | A6 | C | G | G | G | G | T |
| DNA10 | A6 | C | G | G | G | G | T |
| DNA10.2 | A6 | C | G | G | G | G | T |
| DNA17.1 | A6 | C | G | G | G | G | T |
| DNA13.1 | A6 | C | G | G | G | G | T |
| P12.1 | A6 | C | G | G | G | G | T |
| AL67188 | A6 | C | G | G | G | G | T |
| Lucy.1 | A6 | C | G | G | G | G | T |
| DNA18.1 | A8 | C | G | A | A | G | C |
| DNA18.2 | A8 | C | G | A | A | G | C |
| DNA19.2 | A8 | C | G | A | A | G | C |
| DNA3 | A8 | C | G | A | A | G | C |
| DNA3 | A8 | C | G | A | A | G | C |
| P14.1 | A8 | C | G | A | A | G | C |
| P4.1 | A8 | C | G | A | A | G | C |
| P7.1 | A8 | C | G | A | A | G | C |
| DNA11.2 | A9.2 | C | G | G | G | G | C |
| DNA12.2 | A9.1 | C | G | G | G | G | C |
| DNA14.2 | A9.1 | C | G | G | G | G | C |
| DNA16.1 | A9.1 | C | G | G | G | G | C |
| DNA16.2 | A9.1 | C | G | G | G | G | C |
| DNA17.2 | A9.1 | C | G | G | G | G | C |
| DNA2.2 | A9.3 | C | G | G | G | G | C |
| DNA20.1 | A9.1 | C | G | G | G | G | C |
| DNA20.2 | A9.1 | C | G | G | G | G | C |
| DNA4.2 | A9.1 | C | G | G | G | G | C |
| DNA6.2 | A9.1 | C | G | G | G | G | C |
| LucyA22 | A9.1 | C | G | G | G | G | C |
| P12.2 | A9.1 | C | G | G | G | G | C |
| P13.1 | A9.1 | C | G | G | G | G | C |
| P13.2 | A9.1 | C | G | G | G | G | C |
| P14.2 | A9.1 | C | G | G | G | G | C |
| P15.2 | A9.1 | C | G | G | G | G | C |
| P16.2 | A9.1 | C | G | G | G | G | C |
| P17.2 | A9.1 | C | G | G | G | G | C |
| P18.1 | A9.1 | C | G | G | G | G | C |
| P18.2 | A9.2 | C | G | G | G | G | C |
| P19.2 | A9.2 | C | G | G | G | G | C |
| P2.2 | A9.1 | C | G | G | G | G | C |
| P3.1 | A9.1 | C | G | G | G | G | C |
| P3.2 | A9.2 | C | G | G | G | G | C |
| P4.2 | A9.1 | C | G | G | G | G | C |
| P6.1 | A9.1 | C | G | G | G | G | C |
| P7.2 | A9.3 | C | G | G | G | G | C |
| P8.1 | A9.1 | C | G | G | G | G | C |
| P8.2 | A9.1 | C | G | G | G | G | C |
| P9.2 | A9.2 | C | G | G | G | G | C |
| S5.1 | A9.1 | C | G | G | G | G | C |
| DNA11.1 | A10 | G | A | G | G | C | C |
| DNA13.2 | A10 | G | A | G | G | C | C |
| P10.2 | A10 | G | A | G | G | C | C |
| P15.1 | A10 | G | A | G | G | C | C |
| P17.1 | A10 | G | A | G | G | C | C |
| P11.1 | A10 | G | A | G | G | C | C |
| P11.2 | A10 | G | A | G | G | C | C |
| DNA5.2 | A10 | G | A | G | G | C | C |
| DNA2.1 | A10 | G | A | G | G | C | C |
| S1GA | A10 | G | A | G | G | C | C |
| S3.2 | A10 | G | A | G | G | C | C |
| DNA7.2 | A10 | G | A | G | G | C | C |
| LA2 | A7 | G | A | G | G | C | C |

| | SNP | | | | |
|---|---|---|---|---|---|
| | SNPG2314 | SNPG2370 | SNPG2375 | SNPG2449 | SNPG2525 |
| | R | M | W | W | Y |
| AA CHANGE | Q/Q | */C | | | |
| Boleth.1 | G | A | A | A | C |
| Boleth.2 | G | A | A | A | C |
| P19.1 | G | A | A | A | C |
| DNA14.1 | G | A | A | A | C |
| P10.1 | G | A | A | A | C |
| P1.1 | G | A | A | A | C |
| LA1 | G | A | A | A | C |
| P1.2 | G | A | A | A | C |
| P5.2 | G | A | A | A | C |
| AC004204 | G | A | A | A | C |
| BX248310 | G | A | A | A | C |
| AL84544 | G | A | A | A | C |
| S2AG | G | A | A | A | C |
| DNA5.1 | G | A | A | A | C |
| DNA4.1 | G | A | A | A | C |
| DNA7.11 | G | A | A | A | C |
| DNA9.1 | G | A | A | A | C |
| DNA15.1 | G | A | A | A | C |
| DNA15.2 | G | A | A | A | C |
| DNA19.1 | G | A | A | A | C |
| AL662833 | G | A | A | T | C |
| AP000508 | G | A | A | T | C |
| S5.2 | G | A | A | T | C |
| S4.1 | G | A | A | A | C |
| S4.2 | G | A | A | A | C |
| DNA8 | G | A | A | T | C |
| DNA8.2 | G | A | A | T | C |
| DNA12.1 | G | A | A | T | C |
| P2.1 | G | A | A | T | C |
| P16.1 | G | A | A | T | C |
| P5.1 | G | A | T | A | C |
| P9.1 | G | A | T | A | C |
| P6.2 | G | A | T | A | C |
| S1AC | A | A | A | A | C |
| S2CA | A | A | A | A | C |
| S3.1 | A | A | A | A | C |
| DNA1.1 | A | A | A | A | C |
| DNA1.2 | A | A | A | A | C |
| DNA6.1 | A | A | A | A | C |
| DNA9.2 | A | A | A | A | C |
| DNA10 | A | A | A | A | C |
| DNA10.2 | A | A | A | A | C |
| DNA17.1 | A | A | A | A | C |
| DNA13.1 | A | A | A | A | C |
| P12.1 | A | A | A | A | C |
| AL67188 | A | A | A | A | C |
| Lucy.1 | A | A | A | A | C |
| DNA18.1 | G | A | A | A | C |
| DNA18.2 | G | A | A | A | C |
| DNA19.2 | G | A | A | A | C |
| DNA3 | G | A | A | A | C |
| DNA3 | G | A | A | A | C |
| P14.1 | G | A | A | A | C |
| P4.1 | G | A | A | A | C |
| P7.1 | G | A | A | A | C |
| DNA11.2 | G | C | A | A | C |
| DNA12.2 | G | C | A | A | C |
| DNA14.2 | G | C | A | A | C |
| DNA16.1 | G | C | A | A | C |
| DNA16.2 | G | C | A | A | C |
| DNA17.2 | G | C | A | A | C |
| DNA2.2 | G | C | A | A | C |
| DNA20.1 | G | C | A | A | C |
| DNA20.2 | G | C | A | A | C |
| DNA4.2 | G | C | A | A | C |
| DNA6.2 | G | C | A | A | C |
| LucyA22 | G | C | A | A | C |
| P12.2 | G | C | A | A | C |
| P13.1 | G | C | A | A | C |
| P13.2 | G | C | A | A | C |
| P14.2 | G | C | A | A | C |
| P15.2 | G | C | A | A | C |
| P16.2 | G | C | A | A | C |
| P17.2 | G | C | A | A | C |
| P18.1 | G | C | A | A | C |
| P18.2 | G | C | A | A | C |
| P19.2 | G | C | A | A | C |
| P2.2 | G | C | A | A | C |
| P3.1 | G | C | A | A | C |
| P3.2 | G | C | A | A | C |
| P4.2 | G | C | A | A | C |
| P6.1 | G | C | A | A | C |
| P7.2 | G | C | A | A | C |
| P8.1 | G | C | A | A | C |
| P8.2 | G | C | A | A | C |
| P9.2 | G | C | A | A | C |
| S5.1 | G | C | A | A | C |
| DNA11.1 | G | A | A | A | T |
| DNA13.2 | G | A | A | A | T |
| P10.2 | G | A | A | A | T |
| P15.1 | G | A | A | A | T |
| P17.1 | G | A | A | A | T |
| P11.1 | G | A | A | A | T |
| P11.2 | G | A | A | A | T |
| DNA5.2 | G | A | A | A | T |
| DNA2.1 | G | A | A | A | T |
| S1GA | G | A | A | A | T |
| S3.2 | G | A | A | A | T |
| DNA7.2 | G | A | A | A | T |
| LA2 | G | A | A | A | T |

### Example 5

### Association with Psoriasis

Using the data in tables 4-7, the frequency of six of the major alleles in the subgroups listed below was calculated:
Random: Individuals on whom a chirurgic intervention has been performed (DNA), Lucy and Boleth cell lines, sequences from databases.
Controls: Russian individuals who do not suffer from schizophrenia (S).

Psoriatic: Russian individuals suffering from posriasis (P).

TABLE 8

| | | Frequency of the allele (measured | | | | | | |
|---|---|---|---|---|---|---|---|---|
| | | in % in the considered subgroup) | | | | | | Total |
| | | A9 | A6 | A3 | A4 | A8 | A10 | % |
| subgroup | random | 9.1 | 22.7 | 18.1 | 22.7 | 0 | 9.1 | 81.7 |
| | controls | 27.5 | 20 | 17.5 | 7.5 | 12.5 | 12.5 | 97.5 |
| | psoriatic | 52.6 | 2.6 | 5.2 | 5.2 | 7.9 | 13.1 | 86.6 |

Thus it can be concluded that allele A9 is much more frequent in the subgroup corresponding to individuals suffering from psoriasis than in the other subgroups. Accordingly, the presence of allele A9 in an individual indicates that said individual suffers from or is at risk of suffering from psoriasis.

### Example 6

### Alternative Splice forms of UBP8rp

Three different splice variants encoded by the UBP8rp gene SEQ ID No. 52, 54 and 55, were isolated by RT cDNA PCR cloning from skin biopsies from psoriatic patients. These splice variants are expressed from allele A9. Attempts to isolate these three splice variants from individuals who do not possess allele A9 failed. Accordingly, it can be concluded that UBP8rp polynucleotides of SEQ ID No. 52, 54 and 55 are specifically expressed by individuals suffering from posriasis

6.1. Protocol

Preparation of cDNA

Preparation of cDNA was made from RNA extracts of biopsies from patients suffering from psoriasis. The Clontech RT-for-PCR kit was used for this preparation. Both oligo(dT) and random primers were used for each sample for cDNA priming. Approximatively 0.5 to 1 μg RNA was taken per reaction.

Amplification

The nested PCR was performed on 5 μl of each cDNA First couple of primers was primers of SEQ ID Nos. 67 and 68, then the first PCR was diluted 1:50 an d 3111 of dilution reamplified by primers of SEQ ID Nos. 78 and 79, which contain restriction sites.

PCRs were loaded on a PTC-200 apparatus, with the same program for both PCRs: 94° C. 5 min

94° C. 20sec
67° C. 3 min
35 cycles
72° C. 10 min
   The bands obtained by the second PCR were purified by Phe-Chl mix and washed twice on MICROCON-100 columns.

Cloning

Purified PCR bands were digested by BamHI-BgIll. Digestion mix was purified by Chloroform and double wash on MICROCON-100 columns by 500 ul H20.

Cloning was performed in pGEM11Zf vector digested by BamHI, 10 ng of vector digestion was ligated with each total PCR digest in 15 μl mix using 0.5 μl (100U) of T4 DNA Ligase (Biolabs) overnight at 16° C.

Purified by Chloroform and MICROCON-100 ligations were electroporated in competent cells DH10B. White colonies were selected and insert presence was confirmed by PCR with sequencing.

Clones were sequenced with primers of SEQ ID Nos. 70-79. The splicing variants were identified by sequence comparison to genomic DNA of known allelic variants.

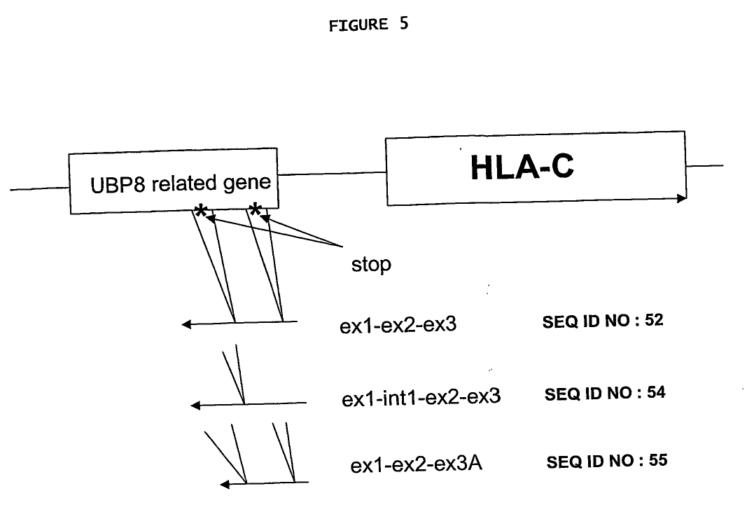

The intronfexon structure of these splice variants is schematized on FIG. 5. SEQ ID NO: 52 encodes the protein of SEQ ID NO: 53. SEQ ID NO: 55 encodes the protein of SEQ ID NO: 56. SEQ ID NO: 54 does not encode any functional protein. Intron 1 being not spliced out, a stop coding is present at the beginning of the open reading frame of SEQ ID NO: 54.

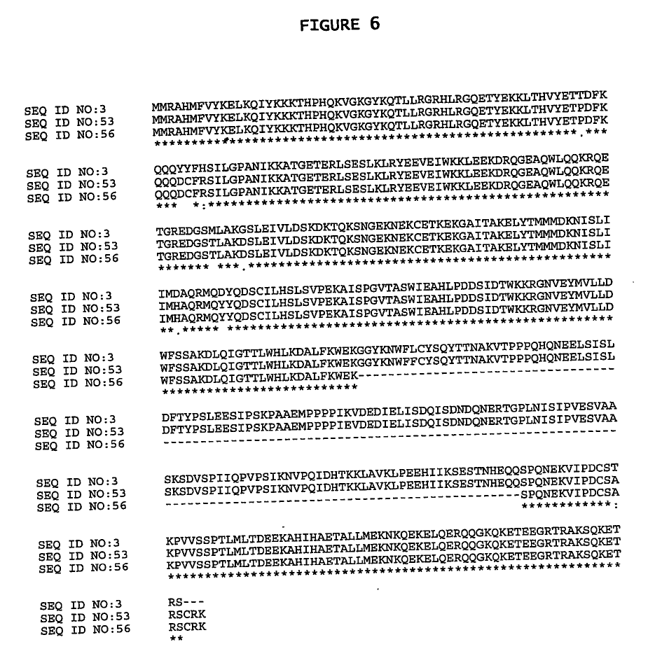

An alignment between SEQ ID Nos. 53, 56 and 2 is shown on FIG. 6.

### Example 7

### Immunologic Analysis of UBP8rp

7.1. Protocols

Tissues and Cells Analyzed

Tissues tested with anti-UBP8rp peptide polyclonal antibodies are the following: skin (human and mice), spinal cord, fetal brain, hypothalamus, parietal lobe, liver, lung, kidney, heart, smooth muscle, skeletal muscle and umbilical cord.

Human primary cultures and cell lines tested with anti-UBP-rel peptide polyclonal antibodies are the following: PBMC, B-lymphocytes, Differentiating keratinocytes, HaCat, HEK293 and HUVEC

Lysis of Cells

Tissues or cells (skin or leucocytes) were lysed one hour at 4° C. in NP40 1%, Tris-HCL 10 mM pH8, PMSF 1 mM, proteases Inhibitor Roche. The lysat was centrifuged at 14000 rpm for 15 minutes at 4° C., and the proteins were quantified by Bradford method.

SDS-PAGE

50 μg of proteins were deposed on 10% acrylamid gel (NuPage). After the run the proteins were transferred on nitrocellulose membrane at 15 V for 30 minutes. The membrane was blocked by TBS-Tween, 5% milk, and the peptide antibody at 1/1000 was added for overnight incubation at room temperature. After four washings the membrane was incubated for 1 hour with anti-rabbit peroxidase immunoglobulin conjugate. After washings the membrane was revealed by ECL.

2D-PAGE (Two-Dimensional Polyacrylamide Gel Electrophoresis)

The first dimension, IEF (isoelectric focusing) was performed in Zoom IPG Strips pH 3-10 by using 8 M urea, 2% CHAPS, 0.5% ampholytes pH3-10, 0.002% bromophenol blue. Isoelectric focusing was performed at 600 V for 7 hours. The Strips were equilibrated in NuPAGE LDS sample buffer (Invitrogen) and the second dimension was performed on 4-12% NuPAGE gel.

ELISA (Enzyme-Linked Immunosorbent Assay)

Wells of microtiter plates (F96 maxisorp NUNC) were coated with 100 μl of peptide in 15 mM Na2CO3 and 35 mM NaHCO3, pH 9.6 for overnight at 4° C. Remaining binding sites were saturated by incubation with 1% BSA in TBST for one hour at room temperature (RT). After washing, antipeptide sera (100 μl per well) diluted In TBS, pH 7.4, containing 0.05% Tween-20 and 1% BSA were Incubated at RT for three hours. After new washing anti rabbit peroxydase immunoglobulin conjugate was added for one hour. Measure of absorbance at 405 nm was done with ABTS substrate.

7.2. Results

A peptide of SEQ ID NO: 57 was used in order to generate anti-UBP8rp polyclonal antibodies. It was shown by SDS page that these antibodies recognize a recombinant UBP8rp protein produced in E. coli. This protein has a molecular weight of about of about 45 kDa.

The presence or absence of the 45 kDa protein corresponding to UBP78p was analyzed by SDS-PAGE in different tissues and samples. The results are shown in tables 9 and 10. Furthermore, the identity of the 45 kDa protein that is detected in whole blood and in skin was confirmed by 2D-PAGE.

TABLE 9

| tissue | presence of 45 kd protein |
|---|---|
| Fetal brain | no |
| skin | yes |
| spinal cord | no |
| liver | no |
| hypothalamus | no |
| muscle | no |
| kidney | no |
| lung | no |
| Nude mouse skin | no |
| Parietal lobe | no |
| Huvec cells | no |

TABLE 10

| | Detection of the 45 kDa |
|---|---|
| sample | UBP8rp protein |
| whole blood cell samples No. 20537 | yes |
| whole blood cell sample No. 20538 | yes |
| whole blood cell sample No. 20454 | yes |
| whole blood cell sample No. 21896 | yes |
| whole blood cell sample No. 21897 | yes |
| Lymphoblasoid cell line Lucy | no |
| Lymphoblasoid cell line No. 13101 | no |

In conclusion, it has been shown that the 45 kDa UBP8rp protein is present in human skin and whole blood cells but not in lymphoblastoid cell lines in culture or any other human tissue studied.

Given that
UBP8rp belongs to the ubiquitin-proteasome pathway, which plays a role in regulation of cell proliferation;
UBP8rp is expressed in the skin and blood cells that both have a sub population of immuno competent cells
the expression level of UBP8rp is higher in keratinocytes under development than in differentiated keratinocytes;
   Specific variant of UBP8rp may confer abnormal proliferation capacities to psoriatic keratinocytes due to its presence in specific fraction of immuno competent cells in psoriatic lesions. Eventually, abnormal proliferation capacities of psoriatic keratinocytes may be linked with deregulation of UBP8 function due to local expression of UBP8rp. Accordingly, inhibiting the UBP8rp protein may be useful for treating psoriasis by preventing pathological cross talk between proliferating and differentiating keratinocytes and skin specific immunocompetent cells.

### Example 8

### Yeast Two-Hybrid Screening

The yeast two-hybrid screening with Ubp8rp cDNA as a bait was performed to find out polypeptidic binding partners which can either modulate the functional activity of the UBP8rp polypeptide, or which can modulate the binding of the UBP8rp polypeptide to its natural binding partners and thus indirectly modulate its functional activity.

The yeast two-hybrid screening was performed using material from the MatchMakers system commercialized by Clontech. The co-transformation and the mating experiments were performed as described in the Clontech's manual.

The UBP8rp polynucleotide of SEQ ID NO: 52, which encodes the UBP8rp polypeptide of SEQ ID NO: 53, was inserted into the pGBKT7 vector. The obtained bait constructions were used for the screening of MatchMaker™ cDNA libraries. cDNA libraries obtained from human adult skin, human foetal skin and human bone marrow were explored.

Other explored tissues are human cultured keratinocytes, human lymphocytes and human leukocytes. Bait constructions comprising fragments of SEQ ID NO: 52 are further investigated.

### REFERENCES

1. Altschul et al. (1990) J Mol Biol. 215:403-410
2. Borresen et al. (1988) Mutat Res. 202:77-83
3. Cather et al. (2003) Expert Opin Biol Ther. 3:361-370
4. Dempster et al. (1977) JRSSB, 39:1-38
5. Devereux J et al. (1984) Nucleic Acids Res. 12:387-395
6. Eddy. (1996) Current Opinion in Structural Biology 6:361-365
7. Elbashir et al. (2001) Nature 411:494-498
8. Ellington and Szostak (1990) Nature 346:818-822
9. Excoffier and Slatkin, (1995) Mol Biol Evol. 12:921-927
10. Farber et al. (1974) Arch Dermatol. 109:207-111
11. Fields and Song (1989) Nature. 340:245-6
12. Fredriksson et al. (1978) Dermatologica. 157:238-244
13. Fromont-Racine et al. (1997) Nat Genet. 16:277-282
14. Gnesutta et al. (2001) J Biol Chem. 276:39448-39454
15. Gottlieb et al. (2003) J Drugs Dermatol. 2:260-266
16. Grantham (1974) Science 185:862-864
17. Grompe et al. (1989) Proc Natl Acad Sci USA. 86:5888-5892
18. Kato et al. (2000) J Biol Chem. 275:37481-37487
19. Kim et al. (2003) Anal Biochem. 316:251-258
20. Lee et al. (2000) Am J Hum Genet. 67:1020-1024
21. Leube et al. (1988) J Cell Biol. 106:1249-1261
22. Lessa et al. (1993) Mol Ecol. 2:119-129
23. Livak and Schmittgen (2001) Methods 25:402-408
24. Nair et al. (1997) Hum Mol Genet. 6:1349-1356
25. Naviglio et al. (1998) EMBO J. 17:3241-3250
26. Newton et al. (1989) Nucleic Acids Res. 17:2503-2516
27. Nielsen et al. (1999) Protein Engineering 12:3-9
28. Oka et al. (1999) Hum Mol Genet. 8:2165-2170
29. Oldenburg et al. (1992) Proc Natl Acad Sci USA. 89:5393-5397
30. Orita et al. (1989) Proc Natl Acad Sci USA 86:2766-2770
31. Parmley and Smith (1988) Gene. 73:305-318
32. Pearson (1990) Methods in Enzymology, 183:63-99
33. Pearson and Lipman (1988) Proc Nat Acad Sci USA, 85:2444-2448
34. Ramunsen et al. (1997), Electrophoresis, 18: 588-598
35. Ruano et al. (1990) Proc. Natl. Acad. Sci. USA. 87:6296-6300
36. Sarkar and Sommer, (1991) Biotechniques. 10:436-440
37. Semprini et al. (2002) Hum Genet 111:310-313
38. Smith and Waterman (1981) J Mol Evol. 18:38-46
39. Trembath et al. (1997) Hum Mol Genet. 6:813-820
40. Valadon et al. (1996) J Immunol Methods. 197:171-179
41. Veal et al. (2001) J Med Genet. 38:7-13.
42. Veal et al. (2002) Am J Hum Genet. 71:554-564
43. von Heijne. (1992) J. Mol. Biol. 225:487-494
44. Wen et al. (2003) World J Gastroenterol. 9:1342-1346
45. Wilkinson (1997) FASEB J. 11:1245-1256
46. Wilkinson et al. (1995) Biochemistry 34:14535-14546
47. Wu et al. (1989) Proc. Natl. Acad. Sci. USA. 86:2757-2760
48. Zollner et al. (2002), J Clin Invest. 109:671-679

1-43. (canceled)

44. A composition of matter comprising:
a) an isolated gene comprising introns having a sequence of:
(i) nucleotides 1018 to 1046 of SEQ ID NO: 1; and
(ii) nucleotides 1676 to 1718 of SEQ ID NO: 1;
b) an isolated polynucleotide complementary to a messenger RNA transcribed from an isolated gene comprising introns having a sequence of:
(i) nucleotides 1018 to 1046 of SEQ ID NO: 1; and
(ii) nucleotides 1676 to 1718 of SEQ ID NO: 1;
c) an isolated polynucleotide selected from the group consisting of:
(i) a polynucleotide comprising SEQ ID NO: 2;
(ii) a polynucleotide comprising SEQ ID NO: 52;
(iii) a polynucleotide comprising SEQ ID NO: 54;
(iv) a polynucleotide comprising SEQ ID NO: 55;
(v) an allelic variant of any of (i) to (iv) comprising at least one polymorphic variation compared to any of (i) to (iv) respectively, wherein said polymorphic variation is selected from the group consisting of UBP8rp-related biallelic markers Nos. 1 to 96;

| Biallelic | Position on | Alternative |
|---|---|---|
| marker No. | SEQ ID NO: 1 | nucleotides |
| 1 | 1199 | A/G |
| 2 | 1262 | C/T |
| 3 | 1426 | C/G |
| 4 | 1444 | G/T |
| 5 | 1487 | A/G |
| 6 | 1490 | A/G |
| 7 | 1505 | G/T |
| 8 | 1518 | C/T |
| 9 | 1554 | C/T |
| 10 | 1630 | A/G |
| 11 | 1638 | A/T |
| 12 | 1680 | A/G |
| 13 | 1895 | A/G |
| 14 | 2180 | A/G |
| 15 | 2449 | A/T |
| 16 | 2721 | G/T |
| 17 | 3127 | A/G |
| 18 | 3137 | C/T |
| 19 | 3138 | A/G |
| 20 | 3183 | A/G |
| 21 | 3222 | C/G |
| 22 | 3269 | C/T |
| 23 | 3445 | C/T |
| 24 | 3470 | A/G |
| 25 | 3915 | C/T |
| 26 | 3973 | A/C |
| 27 | 4254 | A/G |
| 28 | 4472 | A/T |
| 29 | 4660 | C/T |
| 30 | 4770 | A/G |
| 31 | 4919 | A/G |
| 32 | 4973 | C/T |
| 33 | 5063 | C/T |
| 34 | 5065 | G/T |
| 35 | 5079 | C/T |
| 36 | 5080 | C/T |
| 37 | 5088 | C/G |
| 38 | 5090 | C/T |
| 39 | 5407 | C/T |
| 40 | 5466 | A/G |
| 41 | 5520 | C/T |
| 42 | 829 | A/G |
| 43 | 856 | A/G |
| 44 | 902 | insertion of G |
| 45 | 908 | insertion of A |
| 46 | 972 | A/G |
| 47 | 975 | A/G |
| 48 | 1006 | C/T |
| 49 | 1018 | A/G |
| 50 | 1048 | A/C |
| 51 | 1056 | C/T |
| 52 | 1069 | G/T |
| 53 | 1073 | A/G |
| 54 | 1079 | A/G |
| 55 | 1108 | A/G |
| 56 | 1154 | A/G |
| 57 | 1181 | A/G |
| 58 | 1236 | A/G |
| 59 | 1263 | A/G |
| 60 | 1274 | A/G |
| 61 | 1319 | G/T |
| 62 | 1334 | A/G |
| 63 | 1444 | G/T |
| 64 | 1466 | C/T |
| 65 | 1489 | A/G |
| 66 | 1508 | C/T |
| 67 | 1521 | G/T |
| 68 | 1543 | A/G |
| 69 | 1687 | A/C |
| 70 | 1707 | deletion of C |
| 71 | 1728 | A/G |
| 72 | 1742 | C/T |
| 73 | 1810 | C/T |
| 74 | 1813 | A/C |
| 75 | 1841 | C/T |
| 76 | 1874 | C/G |
| 77 | 1875 | A/G |
| 78 | 1890 | A/C |
| 79 | 1907 | A/G |
| 80 | 1909 | C/T |
| 81 | 1921 | A/C |
| 82 | 1922 | A/G |
| 83 | 1957 | A/G |
| 84 | 1959 | A/G |
| 85 | 1976 | C/T |
| 86 | 1992 | C/T |
| 87 | 1993 | C/T |
| 88 | 2096 | C/G |
| 89 | 2135 | A/G |
| 90 | 2192 | A/G |
| 91 | 2230 | C/G |
| 92 | 2275 | C/T |
| 93 | 2314 | A/G |
| 94 | 2370 | A/C |
| 95 | 2375 | A/T |
| 96 | 2525 | C/T |

d) a polynucleotide complementary to any of (a) to (c);
e) an isolated polypeptide encoded by:
(1) an isolated gene comprising introns having a sequence of:
(i) nucleotides 1018 to 1046 of SEQ ID NO: 1; and
(ii) nucleotides 1676 to 1718 of SEQ ID NO: 1;
(2) an isolated polynucleotide complementary to a messenger RNA transcribed from an isolated gene comprising introns having a sequence of:
(i) nucleotides 1018 to 1046 of SEQ ID NO: 1; and
(ii) nucleotides 1676 to 1718 of SEQ ID NO: 1; or
(3) an isolated polynucleotide selected from the group consisting of:
(i) a polynucleotide comprising SEQ ID NO: 2;
(ii) a polynucleotide comprising SEQ ID NO: 52;
(iii) a polynucleotide comprising SEQ ID NO: 54;
(iv) a polynucleotide comprising SEQ ID NO: 55;
(v) an allelic variant of any of (i) to (iv) comprising at least one polymorphic variation compared to any of (i) to (iv) respectively, wherein said polymorphic variation is selected from the group consisting of UBP8rp-related biallelic markers Nos. 1 to 96;
f) an isolated polypeptide:
(i) comprising SEQ ID NO:3;
(ii) a polypeptide comprising a span of at least 470 amino acids of SEQ ID NO: 3;
(iii) a polypeptide comprising a span of at least 15 amino acids of SEQ ID NO: 3, wherein said span falls within amino acids 467 to 482 of SEQ ID NO: 3;
(iv) an allelic variant of a polypeptide comprising SEQ ID NO:3; a polypeptide comprising a span of at least 470 amino acids of SEQ ID NO: 3; or a polypeptide comprising a span of at least 15 amino acids of SEQ ID NO: 3, wherein said span falls within amino acids 467 to 482 of SEQ ID NO: 3, said allelic variant comprising at least one polymorphic variation compared to any of said polypeptides respectively, wherein said polymorphic variation is encoded by a codon comprising a UBP8rp-related biallelic marker of the table set forth herein;
(v) a mutein of a polypeptide comprising SEQ ID NO:3; a polypeptide comprising a span of at least 470 amino acids of SEQ ID NO: 3; or a polypeptide comprising a span of at least 15 amino acids of SEQ ID NO: 3, wherein said span falls within amino acids 467 to 482 of SEQ ID NO: 3, wherein the amino acid sequence of said mutein has at least 95%, 96%, 97%, 98% or 99% identity to at least one of the sequences of said polypeptides;
(vi) a mutein of a polypeptide comprising SEQ ID NO:3; a polypeptide comprising a span of at least 470 amino acids of SEQ ID NO: 3; or a polypeptide comprising a span of at least 15 amino acids of SEQ ID NO: 3, wherein said span falls within amino acids 467 to 482 of SEQ ID NO: 3, which mutein is encoded by a nucleic acid which hybridizes to the complement of a DNA sequence encoding any of said polypeptides under highly stringent conditions; or
(vii) a mutein of a polypeptide comprising SEQ ID NO:3; a polypeptide comprising a span of at least 470 amino acids of SEQ ID NO: 3; or a polypeptide comprising a span of at least 15 amino acids of SEQ ID NO: 3, wherein said span falls within amino acids 467 to 482 of SEQ ID NO: 3, wherein any changes in the amino acid sequence of said mutein are conservative amino acid substitutions of the amino acid sequences of said polypeptides;
(viii) a polypeptide comprising SEQ ID NO:53;
(ix) a polypeptide comprising a span of at least 470 amino acids of SEQ ID NO: 53;
(x) a polypeptide comprising a span of at least 15 amino acids of SEQ ID NO: 53, wherein said span falls within amino acids 467 to 485 of SEQ ID NO: 3;
(xi) a mutein of a polypeptide comprising SEQ ID NO:53; a polypeptide comprising a span of at least 470 amino acids of SEQ ID NO: 53; or a polypeptide comprising a span of at least 15 amino acids of SEQ ID NO: 53, wherein said span falls within amino acids 467 to 485 of SEQ ID NO: 3, wherein the amino acid sequence of said mutein has at least 95%, 96%, 97%, 98% or 99% identity to at least one of the polypeptide sequences;
(xii) a mutein of a polypeptide comprising SEQ ID NO:53; a polypeptide comprising a span of at least 470 amino acids of SEQ ID NO: 53; or a polypeptide comprising a span of at least 15 amino acids of SEQ ID NO: 53, wherein said span falls within amino acids 467 to 485 of SEQ ID NO: 3, which mutein is encoded by a nucleic acid which hybridizes to the complement of a DNA sequence encoding any of a polypeptide comprising SEQ ID NO:53; a polypeptide comprising a span of at least 470 amino acids of SEQ ID NO: 53; or a polypeptide comprising a span of at least 15 amino acids of SEQ ID NO: 53, wherein said span falls within amino acids 467 to 485 of SEQ ID NO: 3 under highly stringent conditions; and
(xiii) a mutein of a polypeptide comprising SEQ ID NO:53; a polypeptide comprising a span of at least 470 amino acids of SEQ ID NO: 53; or a polypeptide comprising a span of at least 15 amino acids of SEQ ID NO: 53, wherein said span falls within amino acids 467 to 485 of SEQ ID NO: 3, wherein any changes in the amino acid sequence of said mutein are conservative amino acid substitutions of the amino acid sequences of said polypeptide;
(xiv) a polypeptide comprising SEQ ID NO:56;
(xv) a polypeptide comprising a span of at least 270 amino acids of SEQ ID NO: 56;
(xvi) a polypeptide comprising a span of at least 15 amino acids of SEQ ID NO: 56, wherein said span comprises amino acids 266 and 267 of SEQ ID NO: 56;
(xvii) a mutein of a polypeptide comprising SEQ ID NO:56; a polypeptide comprising a span of at least 270 amino acids of SEQ ID NO: 56; or a polypeptide comprising a span of at least 15 amino acids of SEQ ID NO: 56, wherein said span comprises amino acids 266 and 267 of SEQ ID NO: 56, wherein the amino acid sequence of said mutein has at least 95%, 96%, 97%, 98% or 99% identity to at least one of the polypeptide sequences;
(xviii) a mutein of a polypeptide comprising SEQ ID NO:56; a polypeptide comprising a span of at least 270 amino acids of SEQ ID NO: 56; or a polypeptide comprising a span of at least 15 amino acids of SEQ ID NO: 56, wherein said span comprises amino acids 266 and 267 of SEQ ID NO: 56, which mutein is encoded by a nucleic acid which hybridizes to the complement of a DNA sequence encoding any one of a polypeptide comprising SEQ ID NO:56; a polypeptide comprising a span of at least 270 amino acids of SEQ ID NO: 56; or a polypeptide comprising a span of at least 15 amino acids of SEQ ID NO: 56, wherein said span comprises amino acids 266 and 267 of SEQ ID NO: 56 under highly stringent conditions; or
(xix) a mutein of a polypeptide comprising SEQ ID NO:56; a polypeptide comprising a span of at least 270 amino acids of SEQ ID NO: 56; or a polypeptide comprising a span of at least 15 amino acids of SEQ ID NO: 56, wherein said span comprises amino acids 266 and 267 of SEQ ID NO: 56, wherein any changes in the amino acid sequence of said mutein are conservative amino acid substitutions of the amino acid sequence of said polypeptide;
(g) an expression vector comprising:
A) an isolated gene comprising introns having a sequence of:
(i) nucleotides 1018 to 1046 of SEQ ID NO: 1; and
(ii) nucleotides 1676 to 1718 of SEQ ID NO: 1;
B) an isolated polynucleotide complementary to a messenger RNA transcribed from an isolated gene comprising introns having a sequence of:
(i) nucleotides 1018 to 1046 of SEQ ID NO: 1; and
(ii) nucleotides 1676 to 1718 of SEQ ID NO: 1; or
C) an isolated polynucleotide selected from the group consisting of:
(i) a polynucleotide comprising SEQ ID NO: 2;
(ii) a polynucleotide comprising SEQ ID NO: 52;
(iii) a polynucleotide comprising SEQ ID NO: 54;
(iv) a polynucleotide comprising SEQ ID NO: 55;
(v) an allelic variant of any of (i) to (iv) comprising at least one polymorphic variation compared to any of (i) to (iv) respectively, wherein said polymorphic variation is selected from the group consisting of UBP8rp-related biallelic markers Nos. 1 to 96; or
(vi) a polynucleotide complementary to any of (A) to (C);
(h) a host cell comprising an expression vector comprising:
A) an isolated gene comprising introns having a sequence of:
(i) nucleotides 1018 to 1046 of SEQ ID NO: 1; and
(ii) nucleotides 1676 to 1718 of SEQ ID NO: 1;
B) an isolated polynucleotide complementary to a messenger RNA transcribed from an isolated gene comprising introns having a sequence of:
(i) nucleotides 1018 to 1046 of SEQ ID NO: 1; and
(ii) nucleotides 1676 to 1718 of SEQ ID NO: 1; or
C) an isolated polynucleotide selected from the group consisting of:
(i) a polynucleotide comprising SEQ ID NO: 2;
(ii) a polynucleotide comprising SEQ ID NO: 52;
(iii) a polynucleotide comprising SEQ ID NO: 54;
(iv) a polynucleotide comprising SEQ ID NO: 55;
(v) an allelic variant of any of (i) to (iv) comprising at least one polymorphic variation compared to any of (i) to (iv) respectively, wherein said polymorphic variation is selected from the group consisting of UBP8rp-related biallelic markers Nos. 1 to 96; or
(vi) a polynucleotide complementary to any of (A) to (C); or
(i) an isolated antibody that binds to a polypeptide comprising:
(i) SEQ ID NO:3;
(ii) a polypeptide comprising a span of at least 470 amino acids of SEQ ID NO: 3;
(iii) a polypeptide comprising a span of at least 15 amino acids of SEQ ID NO: 3, wherein said span falls within amino acids 467 to 482 of SEQ ID NO: 3;
(iv) an allelic variant of any of (i) to (iii) comprising at least one polymorphic variation compared to any of (i) to (iii) respectively, wherein said polymorphic variation is encoded by a codon comprising a UBP8related biallelic marker of the table set forth herein;
(v) a mutein of any of (i) to (iii), wherein the amino acid sequence has at least 95%, 96%, 97%, 98% or 99% identity to at least one of the sequences in (i) to (iii);
(vi) a mutein of any of (i) to (iii) which is encoded by a nucleic acid which hybridizes to the complement of a DNA sequence encoding any of (i) to (iii) under highly stringent conditions; or
(vii) a mutein of any of (i) to (iii) wherein any changes in the amino acid sequence are conservative amino acid substitutions of the amino acid sequences in (i) to (iii);
(viii) a polypeptide comprising SEQ ID NO:53;
(ix) a polypeptide comprising a span of at least 470 amino acids of SEQ ID NO: 53;
(x) a polypeptide comprising a span of at least 15 amino acids of SEQ ID NO: 53, wherein said span falls within amino acids 467 to 485 of SEQ ID NO: 3;
(xi) a mutein of a polypeptide comprising SEQ ID NO:53; a polypeptide comprising a span of at least 470 amino acids of SEQ ID NO: 53; or a polypeptide comprising a span of at least 15 amino acids of SEQ ID NO: 53, wherein said span falls within amino acids 467 to 485 of SEQ ID NO: 3, wherein the amino acid sequence of said mutein has at least 95%, 96%, 97%, 98% or 99% identity to at least one of the polypeptide sequences;
(xii) a mutein of a polypeptide comprising SEQ ID NO:53; a polypeptide comprising a span of at least 470 amino acids of SEQ ID NO: 53; or a polypeptide comprising a span of at least 15 amino acids of SEQ ID NO: 53, wherein said span falls within amino acids 467 to 485 of SEQ ID NO: 3, which mutein is encoded by a nucleic acid which hybridizes to the complement of a DNA sequence encoding any of a polypeptide comprising SEQ ID NO:53; a polypeptide comprising a span of at least 470 amino acids of SEQ ID NO: 53; or a polypeptide comprising a span of at least 15 amino acids of SEQ ID NO: 53, wherein said span falls within amino acids 467 to 485 of SEQ ID NO: 3 under highly stringent conditions; and
(xiii) a mutein of a polypeptide comprising SEQ ID NO:53; a polypeptide comprising a span of at least 470 amino acids of SEQ ID NO: 53; or a polypeptide comprising a span of at least 15 amino acids of SEQ ID NO: 53, wherein said span falls within amino acids 467 to 485 of SEQ ID NO: 3, wherein any changes in the amino acid sequence of said mutein are conservative amino acid substitutions of the amino acid sequences of said polypeptide;
(xiv) a polypeptide comprising SEQ ID NO:56;
(xv) a polypeptide comprising a span of at least 270 amino acids of SEQ ID NO: 56;
(xvi) a polypeptide comprising a span of at least 15 amino acids of SEQ ID NO: 56, wherein said span comprises amino acids 266 and 267 of SEQ ID NO: 56;
(xvii) a mutein of a polypeptide comprising SEQ ID NO:56; a polypeptide comprising a span of at least 270 amino acids of SEQ ID NO: 56; or a polypeptide comprising a span of at least 15 amino acids of SEQ ID NO: 56, wherein said span comprises amino acids 266 and 267 of SEQ ID NO: 56, wherein the amino acid sequence of said mutein has at least 95%, 96%, 97%, 98% or 99% identity to at least one of the polypeptide sequences;
(xviii) a mutein of a polypeptide comprising SEQ ID NO:56; a polypeptide comprising a span of at least 270 amino acids of SEQ ID NO: 56; or a polypeptide comprising a span of at least 15 amino acids of SEQ ID NO: 56, wherein said span comprises amino acids 266 and 267 of SEQ ID NO: 56, which mutein is encoded by a nucleic acid which hybridizes to the complement of a DNA sequence encoding any one of a polypeptide comprising SEQ ID NO:56; a polypeptide comprising a span of at least 270 amino acids of SEQ ID NO: 56; or a polypeptide comprising a span of at least 15 amino acids of SEQ ID NO: 56, wherein said span comprises amino acids 266 and 267 of SEQ ID NO: 56 under highly stringent conditions; or
(xix) a mutein of a polypeptide comprising SEQ ID NO:56; a polypeptide comprising a span of at least 270 amino acids of SEQ ID NO: 56; or a polypeptide comprising a span of at least 15 amino acids of SEQ ID NO: 56, wherein said span comprises amino acids 266 and 267 of SEQ ID NO: 56, wherein any changes in the amino acid sequence of said mutein are conservative amino acid substitutions of the amino acid sequences in said polypeptide.

45. The composition of matter according to claim 44, wherein the expression vector is a gene therapy vector.

46. A method of making a polypeptide, said method comprising the steps of culturing a host cell under conditions suitable for the production of a polypeptide, said host cell comprising an expression vector comprising:
A) an isolated gene comprising introns having a sequence of:
(i) nucleotides 1018 to 1046 of SEQ ID NO: 1; and
(ii) nucleotides 1676 to 1718 of SEQ ID NO: 1;
B) an isolated polynucleotide complementary to a messenger RNA transcribed from an isolated gene comprising introns having a sequence of:
(i) nucleotides 1018 to 1046 of SEQ ID NO: 1; and
(ii) nucleotides 1676 to 1718 of SEQ ID NO: 1; or
C) an isolated polynucleotide selected from the group consisting of:
(i) a polynucleotide comprising SEQ ID NO: 2;
(ii) a polynucleotide comprising SEQ ID NO: 52;
(iii) a polynucleotide comprising SEQ ID NO: 54;
(iv) a polynucleotide comprising SEQ ID NO: 55;
(v) an allelic variant of any of (i) to (iv) comprising at least one polymorphic variation compared to any of (i) to (iv) respectively, wherein said polymorphic variation is selected from the group consisting of UBP8rp-related biallelic markers Nos. 1 to 96; or
(vi) a polynucleotide complementary to any of (A) to (C);

47. The method according to claim 46, further comprising the step of purifying said polypeptide from the culture.

48. The method according to claim 47, further comprising the step of formulating said polypeptide into a pharmaceutical composition.

49. A method of screening a polypeptide as a target for screening for natural binding partners or candidate modulators comprising contacting a polypeptide with a natural binding partner or a candidate modulator and measuring the activity of said polypeptide, said polypeptide comprising:
(i) SEQ ID NO:3;
(ii) a polypeptide comprising a span of at least 470 amino acids of SEQ ID NO: 3;
(iii) a polypeptide comprising a span of at least 15 amino acids of SEQ ID NO: 3, wherein said span falls within amino acids 467 to 482 of SEQ ID NO: 3;
(iv) an allelic variant of any of (i) to (iii) comprising at least one polymorphic variation compared to any of (i) to (iii) respectively, wherein said polymorphic variation is encoded by a codon comprising a UBP8rp-related biallelic marker of the table set forth herein;
(v) a mutein of any of (i) to (iii), wherein the amino acid sequence has at least 95%, 96%, 97%, 98% or 99% identity to at least one of the sequences in (i) to (iii);
(vi) a mutein of any of (i) to (iii) which is encoded by a nucleic acid which hybridizes to the complement of a DNA sequence encoding any of (i) to (iii) under highly stringent conditions; or
(vii) a mutein of any of (i) to (iii) wherein any changes in the amino acid sequence are conservative amino acid substitutions of the amino acid sequences in (i) to (iii);
(viii) a polypeptide comprising SEQ ID NO:53;
(ix) a polypeptide comprising a span of at least 470 amino acids of SEQ ID NO: 53;
(x) a polypeptide comprising a span of at least 15 amino acids of SEQ ID NO: 53, wherein said span falls within amino acids 467 to 485 of SEQ ID NO: 3;
(xi) a mutein of a polypeptide comprising SEQ ID NO:53; a polypeptide comprising a span of at least 470 amino acids of SEQ ID NO: 53; or a polypeptide comprising a span of at least 15 amino acids of SEQ ID NO: 53, wherein said span falls within amino acids 467 to 485 of SEQ ID NO: 3, wherein the amino acid sequence of said mutein has at least 95%, 96%, 97%, 98% or 99% identity to at least one of the polypeptide sequences;
(xii) a mutein of a polypeptide comprising SEQ ID NO:53; a polypeptide comprising a span of at least 470 amino acids of SEQ ID NO: 53; or a polypeptide comprising a span of at least 15 amino acids of SEQ ID NO: 53, wherein said span falls within amino acids 467 to 485 of SEQ ID NO: 3, which mutein is encoded by a nucleic acid which hybridizes to the complement of a DNA sequence encoding any of a polypeptide comprising SEQ ID NO:53; a polypeptide comprising a span of at least 470 amino acids of SEQ ID NO: 53; or a polypeptide comprising a span of at least 15 amino acids of SEQ ID NO: 53, wherein said span falls within amino acids 467 to 485 of SEQ ID NO: 3 under highly stringent conditions;
(xiii) a mutein of a polypeptide comprising SEQ ID NO:53; a polypeptide comprising a span of at least 470 amino acids of SEQ ID NO: 53; or a polypeptide comprising a span of at least 15 amino acids of SEQ ID NO: 53, wherein said span falls within amino acids 467 to 485 of SEQ ID NO: 3, wherein any changes in the amino acid sequence of said mutein are conservative amino acid substitutions of the amino acid sequences of said polypeptide;
(xiv) a polypeptide comprising SEQ ID NO:56;
(xv) a polypeptide comprising a span of at least 270 amino acids of SEQ ID NO: 56;
(xvi) a polypeptide comprising a span of at least 15 amino acids of SEQ ID NO: 56, wherein said span comprises amino acids 266 and 267 of SEQ ID NO: 56;
(xvii) a mutein of a polypeptide comprising SEQ ID NO:56; a polypeptide comprising a span of at least 270 amino acids of SEQ ID NO: 56; or a polypeptide comprising a span of at least 15 amino acids of SEQ ID NO: 56, wherein said span comprises amino acids 266 and 267 of SEQ ID NO: 56, wherein the amino acid sequence of said mutein has at least 95%, 96%, 97%, 98% or 99% identity to at least one of the polypeptide sequences;
(xviii) a mutein of a polypeptide comprising SEQ ID NO:56; a polypeptide comprising a span of at least 270 amino acids of SEQ ID NO: 56; or a polypeptide comprising a span of at least 15 amino acids of SEQ ID NO: 56, wherein said span comprises amino acids 266 and 267 of SEQ ID NO: 56, which mutein is encoded by a nucleic acid which hybridizes to the complement of a DNA sequence encoding any one of a polypeptide comprising SEQ ID NO:56; a polypeptide comprising a span of at least 270 amino acids of SEQ ID NO: 56; or a polypeptide comprising a span of at least 15 amino acids of SEQ ID NO: 56, wherein said span comprises amino acids 266 and 267 of SEQ ID NO: 56 under highly stringent conditions; or
(xix) a mutein of a polypeptide comprising SEQ ID NO:56; a polypeptide comprising a span of at least 270 amino acids of SEQ ID NO: 56; or a polypeptide comprising a span of at least 15 amino acids of SEQ ID NO: 56, wherein said span comprises amino acids 266 and 267 of SEQ ID NO: 56, wherein any changes in the amino acid sequence of said mutein are conservative amino acid substitutions of the amino acid sequences in said polypeptide.

50. The method according to claim 49, wherein said candidate modulator is selected from the group consisting of a natural ligand, a small molecule, an aptamer, an antisense mRNA, a small interfering RNA and an antibody.

51. The method according to claim 49, wherein said modulator is a candidate drug for the treatment of a chronic inflammatory disease.

52. The method according to claim 49, wherein the activity of said polypeptide is assessed by measuring the de-ubiquitinating activity of UBP8 in the presence of said polypeptide.

53. A method of assessing the efficiency of a modulator of a polypeptide for the treatment of psoriasis, said method comprising administering said modulator to an animal model for psoriasis; wherein a determination that said modulator ameliorates a representative characteristic of psoriasis in said animal model indicates that said modulator is a drug for the treatment of psoriasis, said polypeptide comprising:
(i) SEQ ID NO:3;
(ii) a polypeptide comprising a span of at least 470 amino acids of SEQ ID NO: 3;
(iii) a polypeptide comprising a span of at least 15 amino acids of SEQ ID NO: 3, wherein said span falls within amino acids 467 to 482 of SEQ ID NO: 3;
(iv) an allelic variant of any of (i) to (iii) comprising at least one polymorphic variation compared to any of (i) to (iii) respectively, wherein said polymorphic variation is encoded by a codon comprising a UBP8rp-related biallelic marker of the table set forth herein;
(v) a mutein of any of (i) to (iii), wherein the amino acid sequence has at least 95%, 96%, 97%, 98% or 99% identity to at least one of the sequences in (i) to (iii);
(vi) a mutein of any of (i) to (iii) which is encoded by a nucleic acid which hybridizes to the complement of a DNA sequence encoding any of (i) to (iii) under highly stringent conditions;
(vii) a mutein of any of (i) to (iii) wherein any changes in the amino acid sequence are conservative amino acid substitutions of the amino acid sequences in (i) to (iii);
(viii) a polypeptide comprising SEQ ID NO:53;
(ix) a polypeptide comprising a span of at least 470 amino acids of SEQ ID NO: 53;
(x) a polypeptide comprising a span of at least 15 amino acids of SEQ ID NO: 53, wherein said span falls within amino acids 467 to 485 of SEQ ID NO: 3;
(xi) a mutein of a polypeptide comprising SEQ ID NO:53; a polypeptide comprising a span of at least 470 amino acids of SEQ ID NO: 53; or a polypeptide comprising a span of at least 15 amino acids of SEQ ID NO: 53, wherein said span falls within amino acids 467 to 485 of SEQ ID NO: 3, wherein the amino acid sequence of said mutein has at least 95%, 96%, 97%, 98% or 99% identity to at least one of the polypeptide sequences;
(xii) a mutein of a polypeptide comprising SEQ ID NO:53; a polypeptide comprising a span of at least 470 amino acids of SEQ ID NO: 53; or a polypeptide comprising a span of at least 15 amino acids of SEQ ID NO: 53, wherein said span falls within amino acids 467 to 485 of SEQ ID NO: 3, which mutein is encoded by a nucleic acid which hybridizes to the complement of a DNA sequence encoding any of a polypeptide comprising SEQ ID NO:53; a polypeptide comprising a span of at least 470 amino acids of SEQ ID NO: 53; or a polypeptide comprising a span of at least 15 amino acids of SEQ ID NO: 53, wherein said span falls within amino acids 467 to 485 of SEQ ID NO: 3 under highly stringent conditions;
(xiii) a mutein of a polypeptide comprising SEQ ID NO:53; a polypeptide comprising a span of at least 470 amino acids of SEQ ID NO: 53; or a polypeptide comprising a span of at least 15 amino acids of SEQ ID NO: 53, wherein said span falls within amino acids 467 to 485 of SEQ ID NO: 3, wherein any changes in the amino acid sequence of said mutein are conservative amino acid substitutions of the amino acid sequences of said polypeptide;
(xiv) a polypeptide comprising SEQ ID NO:56;
(xv) a polypeptide comprising a span of at least 270 amino acids of SEQ ID NO: 56;
(xvi) a polypeptide comprising a span of at least 15 amino acids of SEQ ID NO: 56, wherein said span comprises amino acids 266 and 267 of SEQ ID NO: 56;
(xvii) a mutein of a polypeptide comprising SEQ ID NO:56; a polypeptide comprising a span of at least 270 amino acids of SEQ ID NO: 56; or a polypeptide comprising a span of at least 15 amino acids of SEQ ID NO: 56, wherein said span comprises amino acids 266 and 267 of SEQ ID NO: 56, wherein the amino acid sequence of said mutein has at least 95%, 96%, 97%, 98% or 99% identity to at least one of the polypeptide sequences;
(xviii) a mutein of a polypeptide comprising SEQ ID NO:56; a polypeptide comprising a span of at least 270 amino acids of SEQ ID NO: 56; or a polypeptide comprising a span of at least 15 amino acids of SEQ ID NO: 56, wherein said span comprises amino acids 266 and 267 of SEQ ID NO: 56, which mutein is encoded by a nucleic acid which hybridizes to the complement of a DNA sequence encoding any one of a polypeptide comprising SEQ ID NO:56; a polypeptide comprising a span of at least 270 amino acids of SEQ ID NO: 56; or a polypeptide comprising a span of at least 15 amino acids of SEQ ID NO: 56, wherein said span comprises amino acids 266 and 267 of SEQ ID NO: 56 under highly stringent conditions; or
(xix) a mutein of a polypeptide comprising SEQ ID NO:56; a polypeptide comprising a span of at least 270 amino acids of SEQ ID NO: 56; or a polypeptide comprising a span of at least 15 amino acids of SEQ ID NO: 56, wherein said span comprises amino acids 266 and 267 of SEQ ID NO: 56, wherein any changes in the amino acid sequence of said mutein are conservative amino acid substitutions of the amino acid sequences in said polypeptide.

54. The method according to claim 53, wherein said representative characteristic is a Psoriasis Area and Severity Index score.

55. The method according to claim 54, wherein a 75% or greater improvement in Psoriasis Area and Severity Index scores (PASI 75) indicates that said modulator is a drug for the treatment of psoriasis.

56. The method according to claim 53, wherein said animal model is a SCID-hu Mouse.

57. A method of genotyping comprising the steps of:
a) isolating a nucleic acid from a biological sample; and
b) detecting the nucleotide present at one or more of the UBP8rp-related biallelic markers selected from the group consisting of biallelic markers Nos. 1, 2, 4, 6, 7, 10, 12-19, 21-30, 31-35 and 37-96.

58. The method according to claim 57, wherein said biological sample is derived from a single individual.

59. The method according to claim 58, wherein the identity of the nucleotides at said biallelic marker is determined for both copies of said biallelic marker present in said individual's genome.

60. The method according to claim 57, wherein said determining is performed by a microsequencing assay.

61. The method according to claim 57, further comprising amplifying a portion of a sequence comprising the biallelic marker prior to said determining step.

62. The method according to claim 61, wherein said amplifying is performed by PCR.

63. The method according to claim 57, further comprising the step of correlating the result of the genotyping steps with a risk of suffering from a chronic inflammatory disease.

64. The method according to claim 63, wherein the presence of allele A9 in said individual indicates that said individual suffers from or is at risk of suffering from said chronic inflammatory disease.

65. The method according to claim 64, wherein said chronic inflammatory disease is psoriasis.
